(19) United States
(12) Patent Application Publication
(10) Pub. No.: US 2025/0333423 A1
(43) Pub. Date: Oct. 30, 2025

(54) SMALL MOLECULE MODULATORS OF GP130 SIGNALING PATHWAYS

(71) Applicant: CarthroniX, Inc., Lancaster, CA (US)

(72) Inventors: Benjamin J. Van Handel, Lancaster, CA (US); Adele Elisa Pasqua, Harlow (GB); Katherine Louise Jones, Saffron (GB)

(21) Appl. No.: 18/858,121

(22) PCT Filed: Apr. 20, 2023
(86) PCT No.: PCT/US2023/019317

§ 371 Date: Oct. 18, 2024

Related U.S. Application Data
(60) Provisional application No. 63/333,102, filed on Apr. 20, 2022.

(51) Int. Cl. C07D 513/04; A61K 31/4155; A61K 31/416; A61K 31/422; A61K 31/423; A61K 31/4245; A61K 31/427; A61K 31/428; A61K 31/437; A61K 31/4375; A61K 31/4439; A61K 31/444; A61K 31/4545; A61K 31/4709; A61K 31/4725; A61K 31/496; A61K 31/497; A61K 31/4985; A61K 31/501; A61K 31/506; A61K 31/519; A61K 31/5377; A61K 31/5383; C07D 231/14; C07D 231/40; C07D 271/113; C07D 401/12; C07D 403/12; C07D 413/04; C07D 413/12; C07D 413/14; C07D 417/12; C07D 471/04; C07D 487/04; C07D 495/04; C07D 519/00

(52) U.S. Cl. CPC C07D 513/04; A61K 31/4155; A61K 31/416; A61K 31/422; A61K 31/423; A61K 31/4245; A61K 31/427; A61K 31/428; A61K 31/437; A61K 31/4375; A61K 31/4439; A61K 31/444; A61K 31/4545; A61K 31/4709; A61K 31/4725; A61K 31/496; A61K 31/497; A61K 31/4985; A61K 31/501; A61K 31/506; A61K 31/519; A61K 31/5377; A61K 31/5383; C07D 231/14; C07D 231/40; C07D 271/113; C07D 401/12; C07D 403/12; C07D 413/04; C07D 413/12; C07D 413/14; C07D 417/12; C07D 471/04; C07D 487/04; C07D 495/04; C07D 519/00

(57) ABSTRACT

Disclosed herein are small molecule compounds, compositions, formulations, and methods of modulating gpl30. Compounds, compositions, and formulations described herein are capable of modulating pro-inflammatory, fibrotic and/or regenerative responses. The disclosure also provides methods for treating or ameliorating disease, disorders and conditions associated with gp130 activity, particularly those associated with inflammatory and degenerative disorders, or combination thereof.

### CROSS-REFERENCES TO RELATED APPLICATION

This application claims the benefit of priority of U.S. Provisional Application No. 63/333,102 filed on Apr. 20, 2022, which is incorporated herein by reference in its entirety and for all purposes.

### FIELD

Provided herein are small molecule modulators of gp130, compositions (e.g., pharmaceutical composition), formulations and methods of using the same. In one aspect, small molecule modulators described herein are capable of modulating pro-inflammatory, fibrotic and/or regenerative responses. The disclosure provides methods for treating or ameliorating disease, disorders and conditions associated with gp130 activity, particularly those associated with inflammatory and degenerative disorders, or combination thereof.

### BACKGROUND

Ubiquitously expressed in the human body, glycoprotein 130 (gp130) is a shared subunit of receptor complexes for at least nine cytokines (IL-6, OSM, LIF, IL-11, CNTF, CLC, IL-27, CT-1, and NP) that mediate highly diverse biological processes. The best characterized facet of this receptor and its associated cytokines is the ability to promote or suppress inflammation and regeneration. Therefore, (dys)regulation of gp130 expression, activation, or associated signaling pathways are implicated in a variety of human diseases, including inflammatory, fibrotic, degenerative and proliferative diseases and conditions such as inflammaging. For example, glycoprotein 130 (gp130) is a common receptor subunit of the interleukin-6 (IL-6) family of cytokine which have been shown to increase keratinocyte proliferation and promote matrix deposition (anabolism) by dermal fibroblasts.

After IL-6 family member cytokine binds to its receptor complex via the classic signaling, it induces intracellular signaling pathways, including phosphorylation of the receptor complex and intracellular proteins, via gp130 homodimerization (Murakami et al., 1993). However, molecules in the receptor complex do not have enzyme catalytic domains in their cytoplasmic region even though the complexes activate tyrosine kinases. The discovery of Janus Kinase (JAK), STAT molecules, and negative regulators such as suppressor of cytokine signaling (SOCS) provides new insights into cytokine signaling and is the basis for understanding the role of cytokines in health and disease.

Researchers have showed that signaling through gp130 can have both context-specific and cytokine-specific effects on anti-inflammatory and antidegenerative outputs (Shkhyan R, Van Handel B, Bogdanov J, et al. Annals of the Rheumatic Diseases 2018; 77:760-769). Specifically, some ligands such as oncostatin M strongly promote MAPK-nuclear factor-KB (NF-κB) activity while promoting comparatively little STAT3 activation, whereas others such as LIF and IL-6 result in activation of both pathways.

The binding of IL-6 to its cell-surface receptor activates JAK, thus phosphorylating the six tyrosine residues in the cytoplasmic domain of gp130. The first pathway of gp130 signaling mediated by five distal tyrosine residues (YXXQ motifs) is involved in STAT3 activation. STAT3 is an important transcription factor that transmits signals to the nucleus as the result of JAK phosphorylation and the activation of cytokine receptor-associated kinases. The cytokine receptor-associated kinases phosphorylate STATs which then, homo- or hetero-dimerize via their SH2 domain and translocate to the nucleus to bind to specific DNA elements to regulate gene expression (Masaaki Murakami et al., Immunity, 2019). STAT3 promotes and regulates the transcription of target genes involved in proliferation, apoptosis, and differentiation. Dysregulation of JAK/STAT pathway is implicated in multiple human skin conditions.

The second pathway is related to binding of SHP2 to Y759 which mediates activation of the ERK MAPK cascade via the adaptor molecules GAB1 and GAB2. Additionally, phosphorylated Y759 also serves as a binding site for SOCS3 (Kubo et al., 2003).

The third pathway of gp130 signaling is mediated by Src family kinase, Yes, which directly associates with gp130 via amino acid residues 812-827 (Taniguchi et al., 2015). Yes activates the Yes-associated protein (YAP)-Notch pathway in response to IL-6 or by an activated form of gp130.

NF-κB is a potent proinflammatory nuclear transcription factor and is considered to be a central mediator of inflammatory response. IL-6 activates NF-κB which in turn triggers pro-inflammatory pathway (Wang L. et al., J Immunol. 2003).

Despite the significant permeability barrier of the stratum corneum, delivery via the skin is a very attractive option. Topical treatment of cutaneous disorders obviously targets the site of disease, thereby minimizing adverse side effects elsewhere within the body. However, mainly due to high hydrophobicity, many active compounds intended for transdermal administration are poorly soluble in water providing a challenge to formulate these small molecules. There exists a need in the art for improved formulations for transdermal administration of such compounds.

Thus, new compounds with increased efficacy, reduced metabolic liability and improved physicochemical properties (solubility, potency, functional groups) are needed for successful clinical outcomes for treating or ameliorating disease, disorders and conditions associated with gp130 activity.

Applicant hereby provides a series of small molecule modulators of gp130 signaling pathway with improved properties, including but not limited to, improved “drug-like” properties.

### SUMMARY

In an aspect, provided is a compound having a structure of Formula (I):

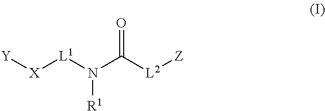

wherein:
X is optionally substituted cycloalkyl, optionally substituted heterocyclyl, optionally substituted heteroaryl, or optionally substituted aryl;
Y is optionally substituted cycloalkyl, optionally substituted heterocyclyl, optionally substituted heteroaryl, or optionally substituted aryl, or is absent;
Z is optionally substituted cycloalkyl, optionally substituted heterocyclyl, optionally substituted heteroaryl, or optionally substituted aryl or optionally substituted alkyl, optionally substituted alkenyl, optionally substituted alkynyl;
L¹ is optionally substituted alkyl, optionally substituted alkenyl, alkynyl, optionally substituted cycloalkyl, optionally substituted cycloalkenyl, —O—, —S—, or —N(R^{1a})—, or is absent;
L² is optionally substituted alkyl, optionally substituted alkenyl, alkynyl, optionally substituted cycloalkyl, optionally substituted cycloalkenyl, —O—, —S—, or —N(R^{1a})—, or is absent;
R¹ is H or C₁-C₆ alkyl;
each R^{1a} is H or C₁-C₆ alkyl;
or
a pharmaceutically acceptable salt thereof.

In certain embodiments, in Formula (I), X suitably may be:

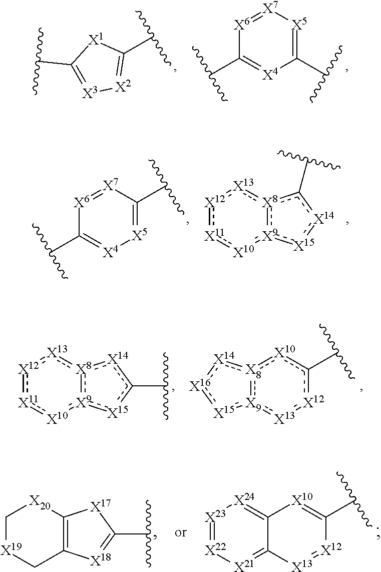

and Y is

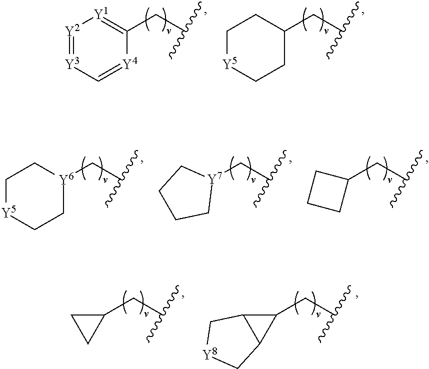

or is absent;
wherein,
   X¹ to X⁸ are each independently —C(R²)═, —N═, —O—, or —S—;
   X¹⁹ and X²⁰ are each independently —C(R²)(R³)—, —N(R⁴)—, —O—, or —S—;
   Y¹, Y², Y³ and Y⁴ are each independently —C(R²)═, or —N═;
   Y⁵ and Y⁸ are each independently —C(R²)(R³)—, —N(R⁴)—, —O—, or —S—;
   Y⁶ and Y⁷ are each independently —C(R²)—, or N;
   R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl,

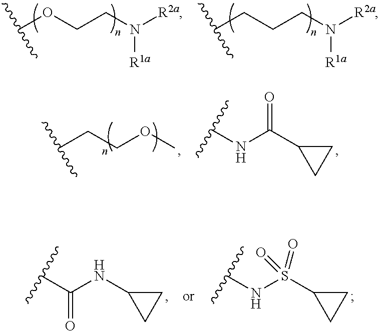

R⁴ is H, (C₁-C₃) alkyl, (C₁-C₃)haloalkyl, (C₁-C₃)alkenyl, (C₁-C₃)alkynyl, (C₁-C₃)alkoxyl

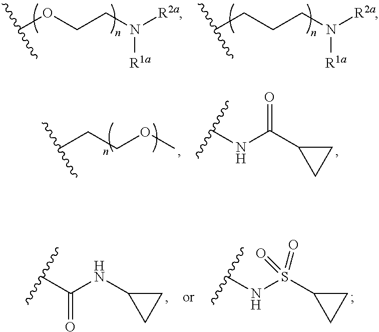

R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl;
v is 0, 1 or 2; and
n is 0, 1, 2, 3, 4, or 5.

In certain embodiments, Z may be:

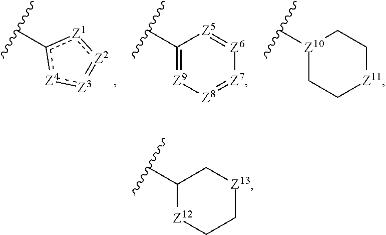

or is absent;
Z¹ to Z⁹ are each independently —C(R²)=, —N═, —O—, or —S—;
Z¹⁰ is —C(R²)— or N;
Z¹¹, Z¹² and Z¹³ are each independently —C(R²)(R³)—, —N(R⁴)—, —O—, or —S—.
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl,

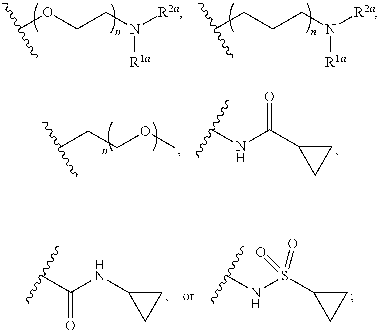

R⁴ is H, (C₁-C₃) alkyl, (C₁-C₃)haloalkyl, (C₁-C₃)alkenyl, (C₁-C₃)alkynyl, (C₁-C₃)alkoxyl,

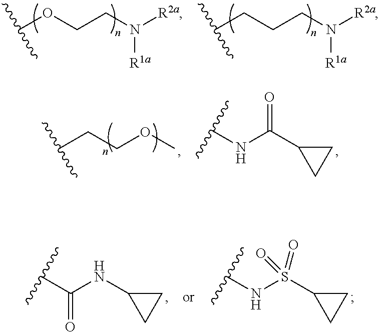

R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl; and
n is 0, 1, 2, 3, 4, or 5.

In an aspect, provided is a compound having a structure of Formula (X-I):

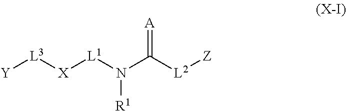

wherein:
A is O or S;
X is optionally substituted cycloalkyl, optionally substituted heterocyclyl, optionally substituted heteroaryl, or optionally substituted aryl;
Y is optionally substituted cycloalkyl, optionally substituted heterocyclyl, optionally substituted heteroaryl, or optionally substituted aryl, or is absent;
Z is optionally substituted cycloalkyl, optionally substituted heterocyclyl, optionally substituted heteroaryl, or optionally substituted aryl or optionally substituted alkyl, optionally substituted alkenyl, optionally substituted alkynyl;
L¹ is optionally substituted alkyl, optionally substituted alkenyl, alkynyl, optionally substituted cycloalkyl, optionally substituted cycloalkenyl, —O—, —S—, or —N(R^{1a})—, or is absent;
L² is optionally substituted alkyl, optionally substituted alkenyl, alkynyl, optionally substituted cycloalkyl, optionally substituted cycloalkenyl, —O—, —S—, or -N(R^{1a})—, or is absent;
L³ is optionally substituted alkyl, optionally substituted alkenyl, alkynyl, optionally substituted cycloalkyl, optionally substituted cycloalkenyl, —C(O)—, NH—C(O)—, —C(O)—NH—, —O—, —S—, or —N(R^{1a})—, or is absent;
R¹ is H or C₁-C₆ alkyl;
each R^{1a} is H or C₁-C₆ alkyl;
or
   a pharmaceutically acceptable salt thereof.

In certain embodiments, in Formula (X-I), X suitably may be:

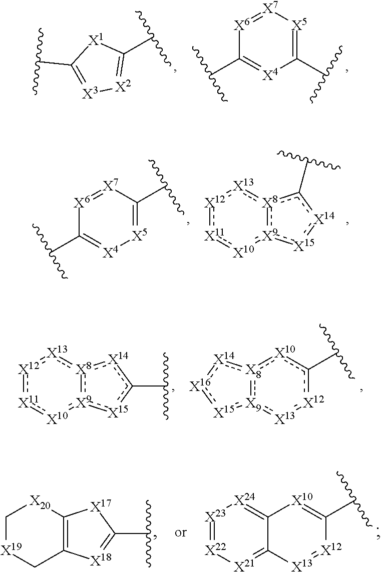

and Y is

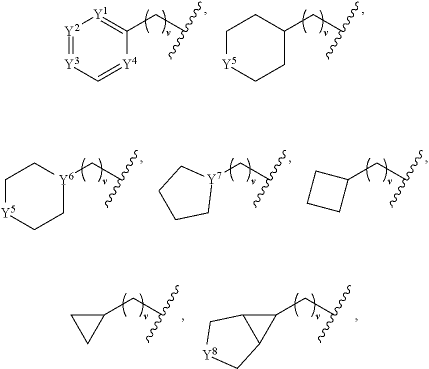

or is absent;
wherein,
   X¹ to X⁸ are each independently —C(R²)=, —N═, —O—, or —S—;
   X¹⁹ and X²⁰ are each independently —C(R²)(R³)—, —N(R⁴)—, —O—, or —S—;
   Y¹, Y², Y³ and Y⁴ are each independently —C(R²)=, or —N═;
   Y⁵ and Y⁸ are each independently —C(R²)(R³)—, —N(R⁴)—, —O—, or —S—;
   Y⁶ and Y⁷ are each independently —C(R²)—, or N;
   R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

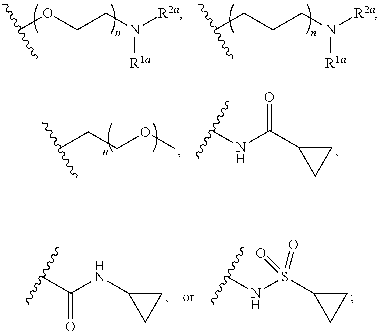

R⁴ is H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, C₃-C₆) branched alkynyl, (C₃-C₆) branched alkoxyl,

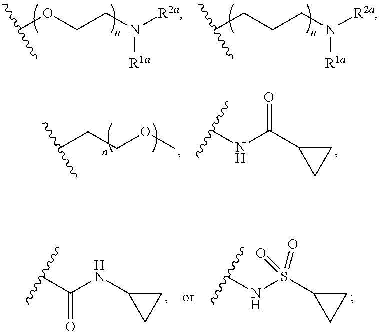

R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl, (C₃-C₆) branched alkyl, (C₃-C₆) branched haloalkyl, (C₃-C₆) branched alkenyl, (C₃-C₆) branched alkynyl, or (C₃-C₆) branched alkoxyl;
v is 0, 1 or 2; and
n is 0, 1, 2, 3, 4, or 5.

In certain embodiments, Z may be:

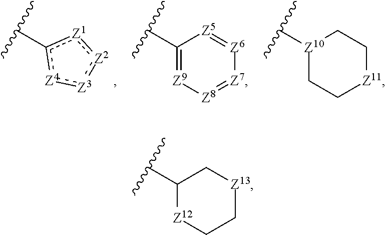

or is absent;
Z¹ to Z⁹ are each independently —C(R²)=, —N═, —O—, or —S—;
Z¹⁰ is —C(R²)— or N;
Z¹¹, Z¹² and Z¹³ are each independently —C(R²)(R³)—, —N(R⁴)—, —O—, or —S—.
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

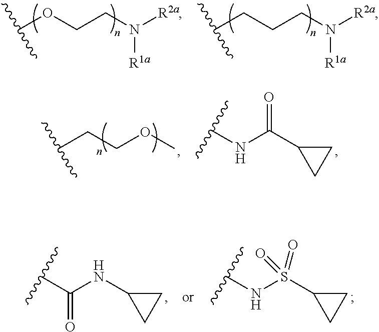

R⁴ is H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl,

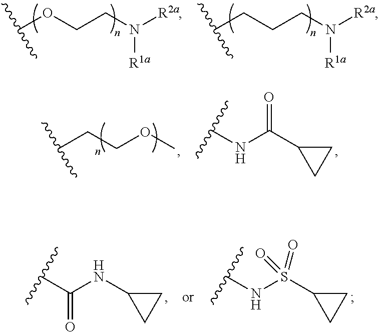

R^{1a} and R^{2a} are each independently H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, or (C₃-C₆) branched alkoxyl; and
n is 0, 1, 2, 3, 4, or 5.

In another aspect, a compound of Formula (X-I) may have a structure of the following Formula (X-I-A):

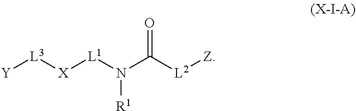

X, Y, Z, L¹, L², L³, and R¹ are as described above.

In another aspect, a compound of Formula (X-I) may have a structure of the following Formula (X-I-B):

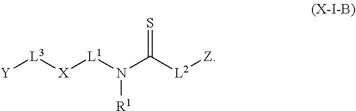

X, Y, Z, L¹, L², L³, and R¹ are as described above.

In certain aspect, L³ is selected from —C(O)—, NH—C(O)—, —C(O)—NH—, —O—, —S—, or —NH— or is absent. In certain aspect, L³ is absent. In certain aspect, L³ is —C(O)—.

In another aspect, a compound of the disclosure may have a structure of the following Formula (II):

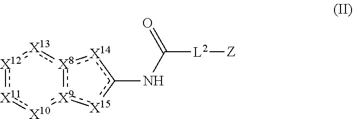

wherein X⁸ to X¹⁵, L¹ and Z are as disclosed above, and pharmaceutically acceptable salts thereof.

In another aspect, a compound of the disclosure may have a structure of the following Formula (IIa):

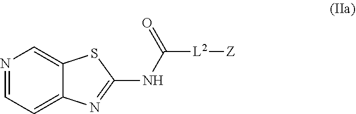

wherein L¹ and Z are as disclosed above, and pharmaceutically acceptable salts thereof.

In another aspect, a compound of the disclosure may have a structure of the following Formula (III):

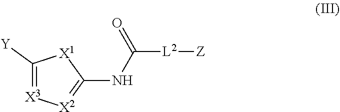

wherein X¹, X², X³, Y, L² and Z are as disclosed above, and pharmaceutically acceptable salts thereof.

In another aspect, a compound of the disclosure may have a structure of the following Formula (IIIa):

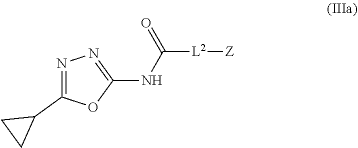

wherein L² and Z are as disclosed above, and pharmaceutically acceptable salts thereof.

In another aspect, a compound of the disclosure may have a structure of the following Formula (IV):

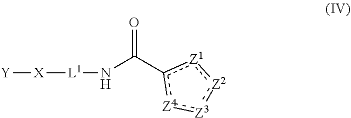

wherein Y, X, L¹, Z¹, Z², Z³ and Z⁴ are as disclosed above, and pharmaceutically acceptable salts thereof.

In another aspect, a compound of the disclosure may have a structure of the following Formula (IVa):

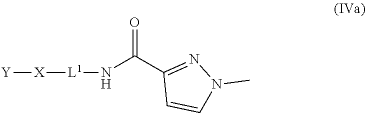

wherein Y, X and L¹ are as disclosed above, and pharmaceutically acceptable salts thereof.

In a further aspect, compounds of the above formulae are provided wherein L¹ is absent.

In a further aspect, compounds of the above formulae are provided wherein L¹ is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —O—, —S—, or —N(R^{1a})—.

In a further aspect, compounds of the above formulae are provided wherein L² is absent.

In a further aspect, compounds of the above formulae are provided wherein L² is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —O—, —S—, or —N(R^{1a})—.

In a further aspect, compounds of the above formulae are provided wherein L¹ is selected from the group consisting of:

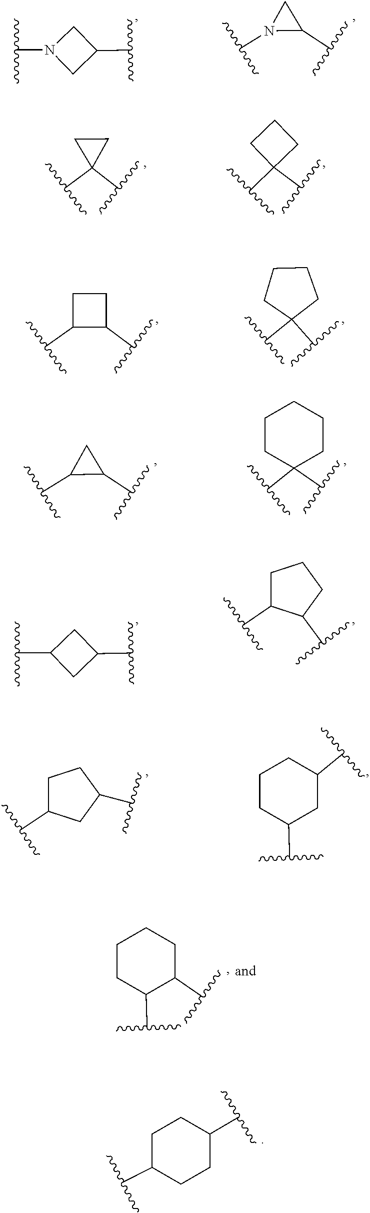

In a further aspect, compounds of the above formulae are provided wherein L² is selected from the group consisting of:

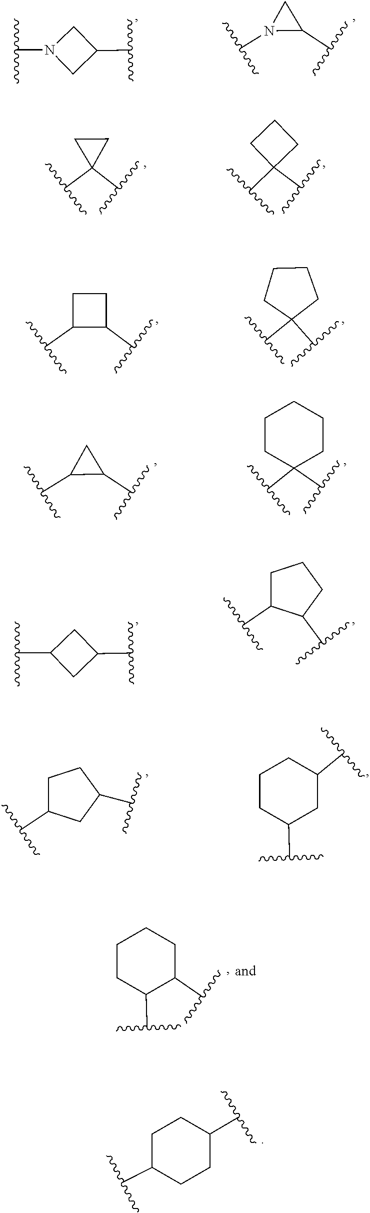

In a further aspect, compounds of the above formulae are provided wherein X is selected from the group consisting of:

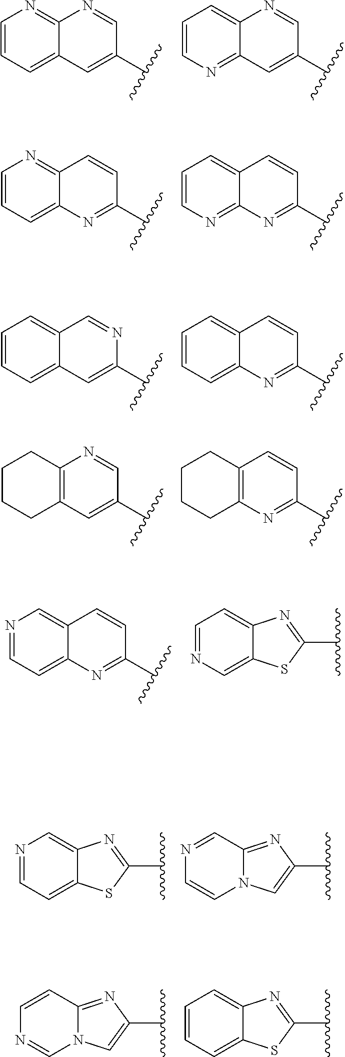

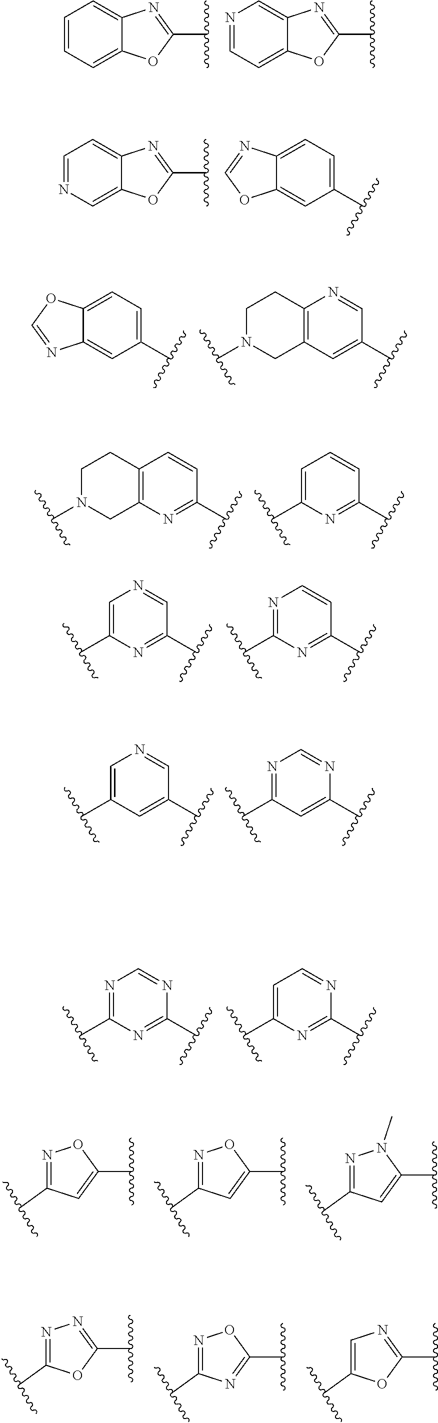

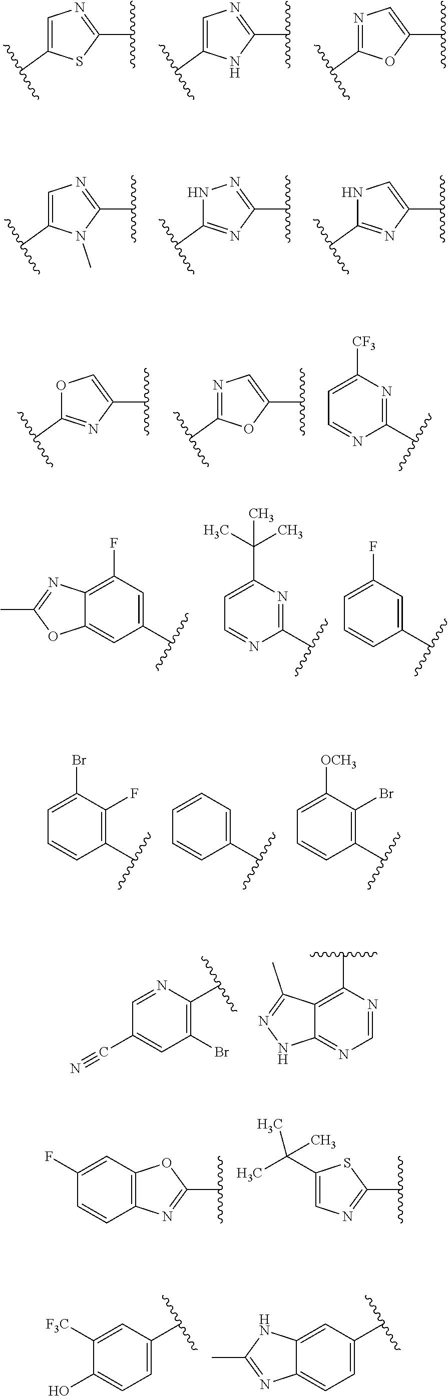

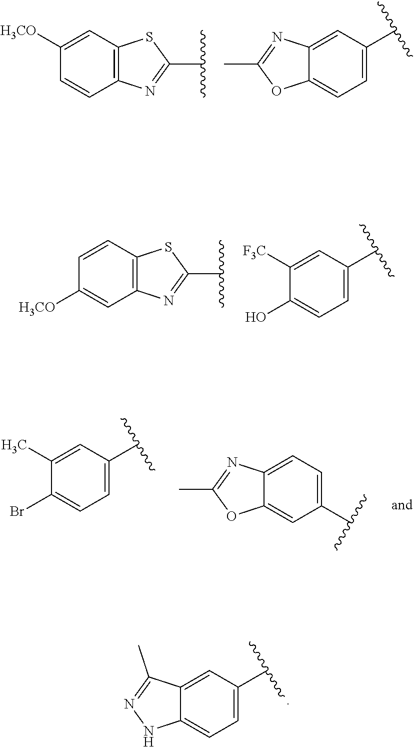

In a further aspect, compounds of the above formulae are provided wherein Z is selected from the group consisting of:

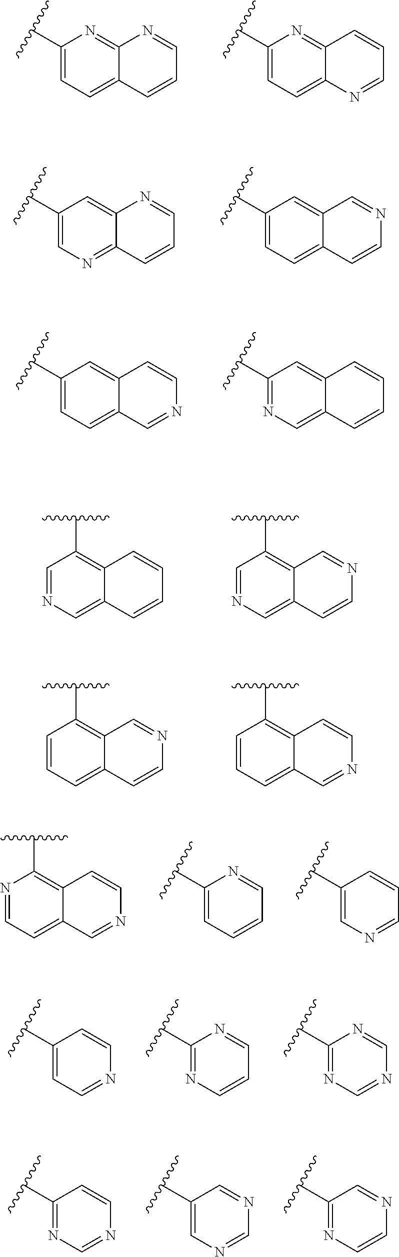

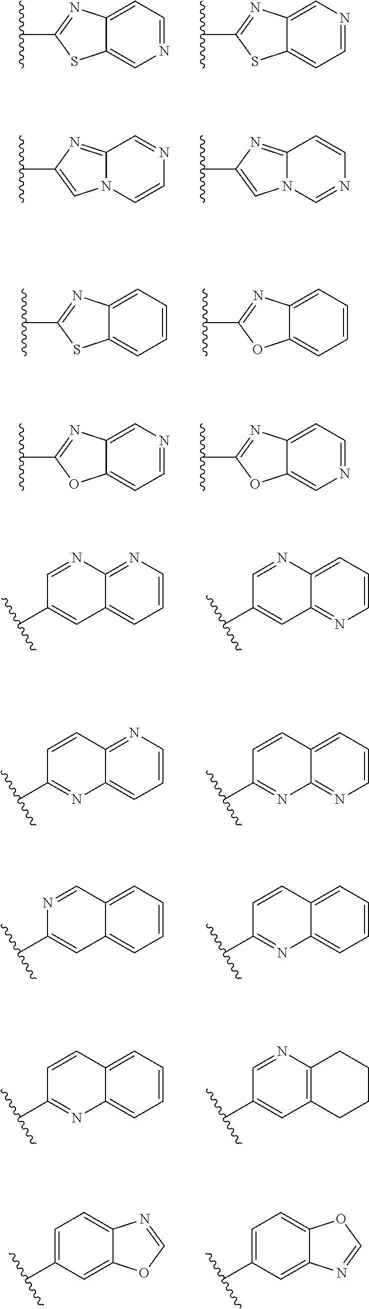

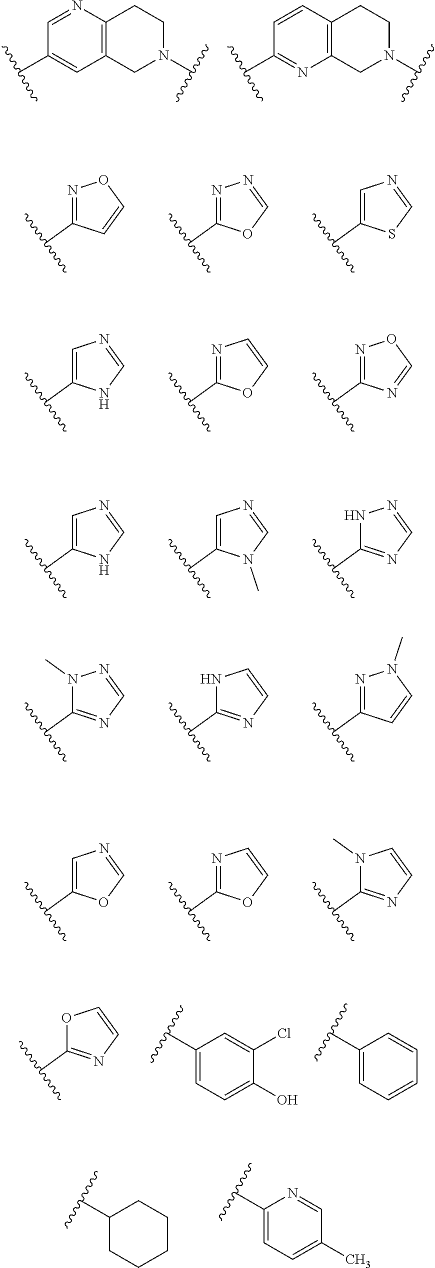

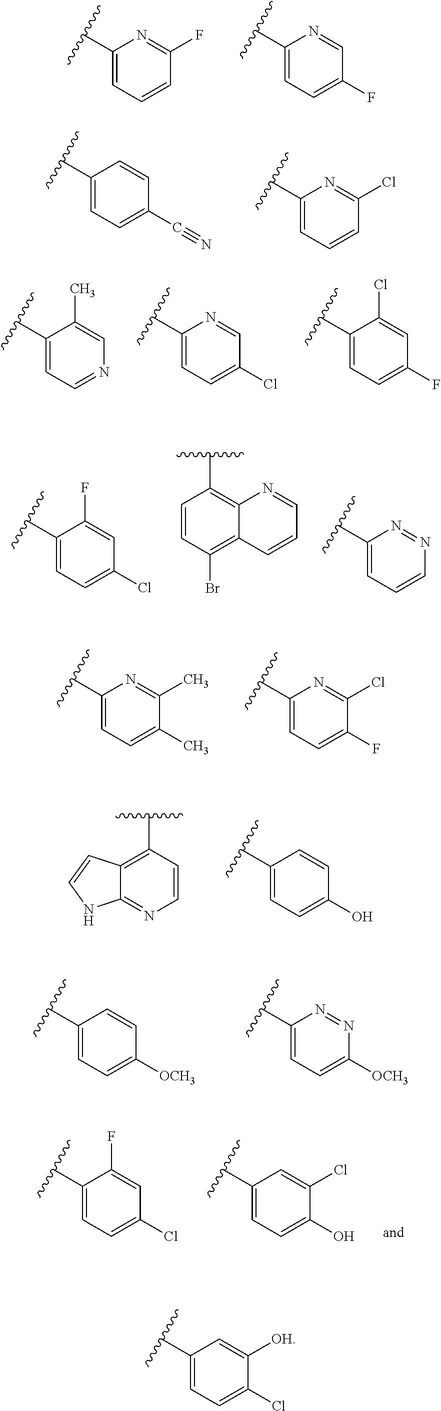

In an aspect, provided is a pharmaceutical composition including one or more compounds as described herein.

In an aspect, provided is a method of treating inflammation, inflammatory disease or disorder, reducing joint pain, preventing joint degeneration or promoting cartilage regeneration in a human subject over the age of 25 in need thereof. The method includes administering to the subject an effective amount of a compound or a composition as described herein.

In an aspect, provided is a method of treating a cell proliferative disease or disorder that is enhanced by gp130 activation in a human subject in need thereof. The method includes administering to the subject an effective amount of a compound or a composition as described herein.

In an aspect, provided is a method of treating or ameliorating a pain condition that is enhanced by gp130 activation in a human subject in need thereof. The method includes administering to the subject an effective amount of a compound or a composition as described herein. In particular, the pain condition is selected from the group consisting of: neuropathic pain, inflammatory pain, headache pain, somatic pain, visceral pain, musckulo-skeletal, craniofacial, other somatic forms of pain and referred pain.

In an aspect, provided is a method of modulating IL-6 family cytokine-mediated inflammatory responses in a cell. The method includes contacting the cell with a compound or a composition described herein.

In an aspect, provided is a composition comprising a pharmaceutically acceptable carrier and a compound or a composition described herein.

In an aspect, provided is a method of treating an acute or chronic inflammatory state. The method includes administering to a subject an effective amount of a compound or a composition as described herein.

In an aspect, provided is a method of decreasing an activated inflammatory pathway in a cell. The method includes contacting the cell with a compound or a composition as described herein.

In an aspect, provided is a method of inhibiting the production or induction of pro-inflammatory genes, cytokines or mediators. The method includes contacting a cell or subject with a compound or a composition as described herein.

In an aspect, provided is a method of inhibiting the production or induction of extracellular matrix degrading enzymes comprising contacting a cell or subject with a compound or a composition as described herein.

In an aspect, provided is a method of modulating STAT3 and/or MYC levels in a cell. The method includes contacting the cell with a compound or a composition as described herein.

In an aspect, provided is a topical formulation including a compound as described herein.

In an aspect, provided is a method of reducing or preventing inflammaging in a target tissue of a human subject in need thereof. The method includes contacting the target tissue with an effective amount of the topical formulation as described herein.

In an aspect, provided is a method of manufacturing a topical formulation of compound in an amount effective to significantly modulate activity or expression of gp130 signaling pathway member in a target population of human skin cells.

In an aspect, provided is a method of treating a subject with skin disorder. The method includes administering to the subject a topical formulation in an amount effective to modulate gp130 signaling in a target population of human skin cells.

In an aspect, provided is a method of cosmetic use. The method includes applying a topical formulation comprising one or more compounds as disclosed hereinto a skin area of a subject.

Other aspects of the invention are disclosed infra.

All publications, patent applications, patents and other reference material mentioned are incorporated by reference in their entirety. In addition, the materials, methods and examples are only illustrative and are not intended to be limiting. The citation of references herein is not to be construed as an admission that the references are prior art to the present invention.

### DETAILED DESCRIPTION

Disclosed herein are modulators of the gp130 signaling pathway and/or downstream pathway members such as STAT-3 and NF-κB.

Also disclosed herein are methods and compositions for treating a subject in need thereof, wherein the subject has a disorder that is modulated by the gp130 signaling pathway and/or downstream pathway members such as STAT-3 and NF-κB.

Also disclosed herein are methods and compositions for treating a subject in need thereof, wherein the subject has an inflammatory disorder that is modulated by the gp130 signaling pathway and/or downstream pathway members such as STAT-3 and NF-κB.

Also disclosed herein are methods and compositions for treating a subject in need thereof, wherein the subject has a proliferative disorder. In embodiments, the proliferative disorder is cancer. In some embodiments, the proliferative disorder is a cancer that is modulated by the gp130 signaling pathway and/or downstream pathway members such as STAT-3 and NF-κB. In some embodiments, the proliferative disorder is a skin cancer. Also disclosed herein are methods and compositions suitable for cosmetically treating a subject with mild to moderate photodamage on human skin cells, e.g., facial skin cells, by applying a novel cream formulation comprising one or more modulators of the gp130 signaling pathway and/or downstream pathway members such as STAT-3 and NF-κB.

The following disclosure provides compositions comprising such modulators, methods of use, and methods of manufacture of a skin care product designed to abrogate these conditions.

In particular, disclosed herein is a study to evaluate the efficacy of a composition disclosed herein when used over the course of 8 weeks by women and men with mild to moderate photodamage on the face.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

### Definitions

All references cited herein are incorporated by reference in their entirety as though fully set forth.

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. Definitions of common terms can be found in Singleton et al., Dictionary of Microbiology and Molecular Biology 3rd ed., J. Wiley & Sons New York, NY (2001); March, Advanced Organic Chemistry Reactions, Mechanisms and Structure 5th ed., J. Wiley & Sons New York, NY (2001); Michael Richard Green and Joseph Sambrook, Molecular Cloning: A Laboratory Manual, 4th ed., Cold Spring Harbor Laboratory Press, Cold Spring Harbor, N.Y., USA (2012); Davis et al., Basic Methods in Molecular Biology, Elsevier Science Publishing, Inc., New York, USA (2012); Jon Lorsch (ed.) Laboratory Methods in Enzymology: DNA, Elsevier, (2013); Frederick M. Ausubel (ed.), Current Protocols in Molecular Biology (CPMB), John Wiley and Sons, (2014); John E. Coligan (ed.), Current Protocols in Protein Science (CPPS), John Wiley and Sons, Inc., (2005); and Ethan M Shevach, Warren Strobe, (eds.) Current Protocols in Immunology (CPI) (John E. Coligan, ADA M Kruisbeek, David H Margulies, John Wiley and Sons, Inc., (2003); each of which provide one skilled in the art with a general guide to many of the terms used in the present application.

As used herein, the singular forms “a,” “an,” and “the” include the plural referents unless the context clearly indicates otherwise. Thus, for example, reference to “a chondrocyte” includes a plurality of such chondrocytes and reference to “an antagonist” includes reference to one or more antagonists or equivalents thereof known to those skilled in the art, and so forth.

The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise. Similarly, “comprise,” “comprises,” “comprising”, “include,” “includes,” and “including” are used interchangeable and not intended to be limiting.

Also, the use of “or” means “and/or” unless stated otherwise.

It is to be further understood that where descriptions of various embodiments use the term “comprising,” those skilled in the art would understand that in some specific instances, an embodiment can be alternatively described using language “consisting essentially of” or “consisting of” The term “about” indicates and encompasses an indicated value and a range above and below that value. In certain embodiments, the term “about” indicates the designated value ±10%, 5%, or ±1%. In certain embodiments, where applicable, the term “about” indicates the designated value(s) ±one standard deviation of that value(s).

The term “alkyl” refers to an organic group that is comprised of carbon and hydrogen atoms that contains single covalent bonds between carbons. Typically, an “alkyl” as used in this disclosure, refers to an organic group that contains 1 to 30 carbon atoms, unless stated otherwise. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkenyl”, refers to an organic group that is comprised of carbon and hydrogen atoms that contains at least one double covalent bond between two carbons. Typically, an “alkenyl” as used in this disclosure, refers to organic group that contains 2 to 30 carbon atoms, unless stated otherwise. While a C2-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of non-conjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkynyl”, refers to an organic group that is comprised of carbon and hydrogen atoms that contains a triple covalent bond between two carbons. Typically, an “alkynyl” as used in this disclosure, refers to organic group that contains 2 to 30 carbon atoms, unless stated otherwise. While a C2-alkynyl can form a triple bond to a carbon of a parent chain, an alkynyl group of three or more carbons can contain more than one triple bond. Where if there is more than 2 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, refers to a conjugated planar ring system with delocalized pi electron clouds that contain only carbon as ring atoms. An “aryl” for the purposes of this disclosure encompasses from 1 to 7 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “heteroalkyl” as used herein by itself or part of another group refers to a stable straight or branched chain hydrocarbon radical containing 1 to 10 carbon atoms and at least two heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In one embodiment, the heteroalkyl contains two oxygen atoms. In another embodiment, the heteroalkyl contains one oxygen and one nitrogen atom.

In another embodiment, the heteroalkyl contains two nitrogen atoms. Non limiting exemplary heteroalkyl groups include —OCH₂CH₂NH₂, —NHCH₂CH₂OCH₃, and —OCH₂CH₂OCH₃.

The term, “Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “cycloalkane”, “cycloalkyl”, as used in this disclosure, refers to an alkyl that contains at least 3 carbon atoms but no more than 12 carbon atoms connected so that it forms a ring. A “cycloalkyl” for the purposes of this disclosure encompass from 1 to 7 cycloalkyl rings, wherein when the cycloalkyl is greater than 1 ring, then the cycloalkyl rings are joined so that they are linked, fused, or a combination thereof. A cycloalkyl may be substituted or unsubstituted, or in the case of more than one cycloalkyl ring, one or more rings may be unsubstituted, one or more rings may be substituted, or a combination thereof.

The term “cycloalkene”, “cycloalkenyl”, as used in this disclosure, refers to an alkene that contains at least 3 carbon atoms but no more than 12 carbon atoms connected so that it forms a ring. A “cycloalkenyl” for the purposes of this disclosure encompass from 1 to 7 cycloalkenyl rings, wherein when the cycloalkenyl is greater than 1 ring, then the cycloalkenyl rings are joined so that they are linked, fused, or a combination thereof. A cycloalkenyl may be substituted or unsubstituted, or in the case of more than one cycloalkenyl ring, one or more rings may be unsubstituted, one or more rings may be substituted, or a combination thereof.

“Disease” or “condition” refer to a state of being or health status of a patient or subject capable of being treated with the compounds/molecules or methods provided herein. Disease as used herein may refer to inflammatory diseases and disorders and immune diseases and disorders such as cartilage degenerative disease, joint surface injury or arthritis (including rheumatoid arthritis), psoriasis, inflammatory bowel disease, aging, lupus, rosacea, fibrosis and the like.

For purposes of this disclosure, the term “extended mixed ring system” refers to a group that is comprised of at least 2 ring structures, but no more than 7 ring structures. An “extended mixed ring system” is comprised of at least one ring functional group that is different from another ring functional group. Examples of ring groups include, but are not limited to, cycloalkyl, cycloalkenyl, cycloalkynyl, aryl, and heterocycle. Each ring may be optionally substituted. The rings comprising the mixed extended ring system may be joined so that they are linked, fused, or a combination thereof.

The term “functional group” or “FG” refers to specific groups of atoms within molecules that are responsible for the characteristic chemical reactions of those molecules. While the same functional group will undergo the same or similar chemical reaction (s) regardless of the size of the molecule it is a part of, its relative reactivity can be modified by nearby functional groups. The atoms of functional groups are linked to each other and to the rest of the molecule by covalent bonds. Examples of FG that can be used in this disclosure, include, but are not limited to, substituted or unsubstituted alkyls, substituted or unsubstituted alkenyls, substituted or unsubstituted alkynyls, substituted or unsubstituted aryls, substituted or unsubstituted hetero-alkyls, substituted or unsubstituted hetero-alkenyls, substituted or unsubstituted hetero-alkynyls, substituted or unsubstituted cycloalkyls, substituted or unsubstituted cycloalkenyls, substituted or unsubstituted hetero-aryls, substituted or unsubstituted heterocycles, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, sulfhydryls, sulfides, disulfides, sulfinyls, sulfos, thiocyanates, isothiocyanates, carbonothioyls, phosphinos, phosphonos, phosphates, Si(OH)₃, Ge(OH)₃, Sn(OH)₃, Si(SH)₄, Ge(SH)₄, AsO₃H, AsO₄H, P(SH)₃, As (SH)₃, SO₃H, Si (OH)₃, Ge(OH)₃, Sn(OH)₃, Si(SH)₄, Ge(SH)₄, Sn(SH)₄, ASO₃H, ASO₄H, P(SH)₃, and As (SH)₃.

The term “gp130” as used herein refers to glycoprotein 130, a cell surface receptor that is expressed ubiquitously in the body and is designated by the gene name IL6ST. Activation of gp130 is essential for several physiological functions, including but not limited to, acute-phase response to injury and infection, fertility, metabolism, haematopoiesis, neuroprotection, anti-angiogenesis, and melanoma and tumor cell suppression. Gp130 is activated by a ligand from the IL-6 family of cytokines, including but not limited to, IL-6, IL-11, leukemia inhibitory factor (LIF). Oncostatin M (OSM), ciliary neurotrophic factor (CNTF), cardiotrophin-1 (CT-1) and cardiotrophin-like cytokine (CLC). Activation of gp130 signaling generally occurs by binding of the ligand to another cell surface receptor, which forms a complex with gp130, thereby activating it. LIF, CT-1, CNTF, OSM and CLC form heterodimers of gp130 and LIFR, whereas OSM may also form a heterodimer of gp130 and OSMR; IL-11 and IL-6 promote formation of heterodimers of gp130 and IL-11Ra and IL-6R, respectively. Therefore, LIF, CT-1, CNTF, OSM and CLC may activate gp130 signaling directly, by binding gp130 first, or indirectly, by binding LIFR/OSMR and then recruiting gp130 to the complex. The ligands of the IL-6 cytokine family trigger the JAK/STAT pathway, the first event of which is the ligand-induced homo- or hetero-dimerization of signal-transducing receptor subunits. All IL-6-type cytokines recruit gp130 to their receptor complexes. They either signal via gp130 alone or in combination with LIFR, OSMR, IL-11Ra or IL-6R, which are all able to activate Jaks and to recruit STAT proteins.

The terms “gp130 receptor,” “gp130,” gp130 protein,” “IL6ST receptor,” “IL6ST” or “IL6ST protein” are here used interchangeably and according to their common, ordinary meaning (e.g., transmembrane protein “glycoprotein 130”) and refer to proteins of the same or similar names and functional fragments and homologs thereof.

The term “hetero-” when used as a prefix, such as, hetero-alkyl, hetero-alkenyl, hetero-alkynyl, or hetero-hydrocarbon, for the purpose of this disclosure refers to the specified hydrocarbon having one or more carbon atoms replaced by non-carbon atoms as part of the parent chain. Examples of such non-carbon atoms include, but are not limited to, N, O, S, Si, Al, B, and P. If there is more than one non-carbon atom in the hetero-based parent chain then this atom may be the same element or may be a combination of different elements, such as N and O.

The term “heterocycle” or “heterocyclyl,”: as used in this disclosure, refers to ring structures that contain at least 1 noncarbon ring atom. A “heterocycle” for the purposes of this disclosure encompass from 1 to 7 heterocycle rings wherein when the heterocycle is greater than 1 ring the heterocycle rings are joined so that they are linked, fused, or a combination thereof. A heterocycle may be a hetero-aryl or nonaromatic, or in the case of more than one heterocycle ring, one or more rings may be nonaromatic, one or more rings may be hetero-aryls, or a combination thereof. A heterocycle may be substituted or unsubstituted, or in the case of more than one heterocycle ring one or more rings may be unsubstituted, one or more rings may be substituted, or a combination thereof. Typically, the noncarbon ring atom is N, O, S, Si, Al, B, or P. In case where there is more than one noncarbon ring atom, these noncarbon ring atoms can either be the same element, or combination of different elements, such as N and O. Examples of heterocycles include, but are not limited to: a monocyclic heterocycle such as, aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2, 5-dihydrofuran tetrahydrofuran, thiophane, piperidine, 1, 2, 3, 6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2, 3, 4, 7-tetrahydro-1H-azepine homopiperazine, 1,3-dioxepane, 4,7-dihydro-1,3-dioxepin, and hexamethylene oxide; and polycyclic heterocycles such as, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1, 2-benzisoxazole, benzothiophene, benzoxazole, benzthiazole, benzimidazole, benztriazole, thioxanthine, carbazole, carboline, acridine, pyrolizidine, and quinolizidine. In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo [2.2.1]heptane and 7-oxabicyclo [2.2.1]heptane.

As used herein, “preventing” refers to the prevention of the disease or condition, e.g., skin condition, in the patient. For example, if an individual at risk of developing a skin disease or other form of skin disorder is treated with the methods of the present invention and does not later develop skin disorder, then the disease has been prevented, at least over a period of time, in that individual.

The term “effective amount” refer to an amount of a compound, formulation, material, or composition, as described herein, effective to achieve a particular cosmetic result.

The term “topical” administration of a formulation disclosed herein refers to application of a formulation to skin of a patient. For purposes of applying a formulation, topical application to the skin shall include application to the stratum corneum. The topical formulation described herein is able to facilitate delivery of an active ingredient through the epidermis by carrying the component having an active ingredient activity past the cells of the epidermis and through the basement membrane into the dermis.

The term “subject” is intended to include living organisms in which an application of the compositions and methods disclosed herein is beneficial in abrogating at least one sign of aging or at least one medical skin condition.

The term “cosmetic” refers to cosmetic preparations which are used to improve looks or the way a person feels about themselves; cosmetic products must deliver visible results without side effects and should appear natural.

The term “chelator” or “chelating agent” refers to any molecule or moiety that is capable of forming a complex (i.e., “chelates”) with a metal ion. In certain exemplary embodiments, a chelator refers to any molecule or moiety that “binds” to a metal ion, in solution, making it unavailable for use in chemical/enzymatic reactions. Chelators generally have two or more unshared electron pairs that can be used to donate to a metal ion. Metal ions are usually coordinated to the chelator by two or more pairs of electrons.

The term “prophylaxis” as used herein means the prevention of or protective treatment for a disease or disease state.

Throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “modulator” refers to a composition that increases or decreases the level of a target molecule or the function of a target molecule or the physical state of the target of the molecule relative to the absence of the modulator.

The term “Inflammation” refers to a complex biological response of a body to a stimulus (e.g., a pathogen, cellular damage or an irritant). Inflammation, when prolonged, can lead to an inflammatory disease or disorder. Factors elicited during an inflammatory reaction include pro-inflammatory cytokines (e.g., TNF-α, IL-1, IFN-gamma, MCP-1, IL-6), cellular migration (e.g., monocytes, macrophages, lymphocytes, plasma cells) and serum proteins (e.g., serum amyloid A (SAA) and serum amyloid P (SAP)). Inflammation can be local (e.g., vascular inflammation) or systemic.

“Inflammatory disorder” or “inflammatory disease” refers to a condition characterized by inflammation in a cell, tissue or body. Inflammatory diseases and disorders include, but are not limited to, hypersensitivities (e.g., allergies), asthma, autoimmune disease (e.g., rheumatoid and osteo arthritis, lupus, multiple sclerosis), cancer, diabetes, inflammatory bowel disease (IBD) or cardiovascular disease (e.g., atherosclerosis), NAFLD, NASH, hepatitis, fibrosis, and cirrhosis.

“Inflammaging” as described herein refers to inflammation, particularly of skin, as a biological response of a body tissue to both environmental challenges such as sun and wind, and internal drivers such as diet, alcohol consumption and smoking, and other potentially harmful stimuli, that promotes detrimental biological responses that result in visible signs of aging such as the appearance of fine lines and wrinkles, hyperpigmentation and increased laxity. The harmful stimuli can include but are not limited to pathogens, bacteria, viruses, fungi, damaged cells and other irritants that are known to those skilled in the art. While inflammation can be a protective immune response that can involve, for example, immune cells, white blood cells, blood vessels, molecular mediators, and other small molecules. Signs of inflammation can include but is not limited to pain, heat, swelling, and/or loss of function. Inflammation can be acute or chronic.

The term “mixed ring system” refers to optionally substituted ring structures that contain at least two rings, and wherein the rings are joined together by linking, fusing, or a combination thereof. A mixed ring system comprises a combination of different ring types, including cycloalkyl, cycloalkenyl, aryl, and heterocycle.

The term “pharmaceutically acceptable” as in pharmaceutically acceptable salt or pharmaceutically acceptable counter ion, refers to compounds, salts, or ions that are tolerated by a subject for topical, or internal use.

The term “pharmaceutically acceptable salt” refers to making a salt formation of a compound disclosed herein. Salt formation can be used as a means of varying the properties of the compounds disclosed herein, for example, to increase or decrease solubility of the compounds, to improve stability of the compounds, to reduce toxicity of the compounds, and/or to reduce the hygroscopicity of the compounds. There are a wide range of chemically diverse acids and bases, with a range of pKa values, molecular weights, solubilities and other properties, that can used for making pharmaceutically acceptable salts of the compounds disclosed herein. Examples of pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the disclosure can form pharmaceutically acceptable salts with various amino acids. Examples of pharmaceutically acceptable base addition salts include, but are not limited to, aluminum, calcium, lithium, magnesium, potassium, sodium, zinc, and diethanolamine salts. For additional examples of pharmaceutical salts that can used to practice this disclosure, see P. H. Stahl and C. G. Wermuth (eds.), Pharmaceutical Salts: Properties, Selection, and Use (2d ed. 2011) Wiley and Sons Publisher, ISBN: 978-3-90639-051-2.

The term “pharmaceutically acceptable counter ion” either refers to pharmaceutically acceptable cations including, but not limited to, alkali metal cations (e.g., Li⁺, Na⁺, K⁺), alkaline earth metal cations (e.g., Ca²⁺, Mg²⁺), non-toxic heavy metal cations and ammonium (NH4⁺) and substituted ammonium (N(R′)4⁺, where R′ is hydrogen, alkyl, or substituted alkyls, i.e., including, methyl, ethyl, or hydroxyethyl, specifically, trimethyl ammonium, triethyl ammonium, and triethanol ammonium cations); or pharmaceutically-acceptable anions including, but not limited to, halides (e.g., Cl″, Br″), sulfate, acetates (e.g., acetate, trifluoroacetate), ascorbates, aspartates, benzoates, citrates, and lactate.

A “subject” generally refers to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

The term “substantially” as used to modify a term means that the modified term includes minor variations in size, purity, structure and the like by only a minor amount. Accordingly, “substantially homogenous in size” means that the material does not vary by more than 1%, 5%, 10%, 20% or 30% (or any value there between) in size from an average size.

The term “substituted” with respect to heterocycles, and the like, refers to structures wherein the parent chain contains one or more substituents.

The term “substituent” refers to an atom or group of atoms substituted in place of a hydrogen atom. For purposes of this disclosure, a substituent would include deuterium atoms.

The term, “therapeutically effective amount,” refers to an amount of a compound, molecule or composition of the disclosure that reduces a symptom or symptoms (and grammatical equivalents of this phrase) or the severity of or frequency of the symptom(s), or elimination of the symptom (s) associated with a disease or disorder to be treated.

The term, “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “unsubstituted” with respect to hydrocarbons, heterocycles, and the like, refers to structures wherein the parent chain contains no substituents.

The term “optionally substituted” with respect to hydrocarbons, heterocycles, and the like, refers to structures wherein the parent chain either contains substituents or contains no substituents. As a non-limiting example, a substituent, as used herein by itself or as part of another group refers to an alkyl group that is either unsubstituted or substituted with one, two, or three substituents, wherein each substituent is independently alkyl, hydroxy, halo, amino, alkylamino, aminoalkyl, nitro, haloalkoxy, aryloxy, aralkyloxy, alkylthio, thioalkyl, sulfonamido, alkylcarbonyl, arylcarbonyl, alkylsulfonyl, arylsulfonyl, ureido, guanidino, carbamate, carboxy, alkoxycarbonyl, and carboxyalkyl.

In one embodiment, the term “modulation” refers to increase and/or induction and/or promotion and/or activation. In an alternative embodiment, the term “modulation” refers to reduction and/or reduction and/or inhibition.

In one embodiment, the term “modulate” refers to upregulation. In an alternative embodiment, the term “modulate” refers to downregulation.

The term “small molecule” refers to a molecule that has a biological effect and has a molecular weight of less than 10000 Daltons. In some embodiments, the small molecule has a molecular weight of less than 5000 Daltons. In some embodiments, the small molecule has a molecular weight of less than 2500 Daltons. In some embodiments, small molecules have a molecular weight of less than 1000 Daltons. In some embodiments, small molecules have a molecular weight of less than 800 Daltons. In some embodiments, small molecules have a molecular weight of less than 500 Daltons.

As used herein, “compound” and “small molecule” are used interchangeable and not intended to be limiting.

The term “pain,” as used here, means any unpleasant sensory experience, usually associated with a physical disorder. The physical disorder may or may not be apparent to a clinician. Pain is of two types: chronic and acute. An “acute pain” is a pain of short duration having a sudden onset. One type of acute pain, for example, is cutaneous pain felt on injury to the skin or other superficial tissues, such as caused by a cut or a burn. Cutaneous nociceptors terminate just below the skin, and due to the high concentration of nerve endings, produce a well-defined, localized pain of short duration. “Chronic pain” is a pain other than an acute pain. Chronic pain includes neuropathic pain, inflammatory pain, headache pain, somatic pain visceral pain and referred pain.

As used herein, a wavy line intersecting another line that is connected to an atom indicates that this atom is covalently bonded to another entity that is present but not being depicted in the structure. A wavy line that does not intersect a line but is connected to an atom indicates that this atom is interacting with another atom by a bond or some other type of identifiable association.

A bond indicated by a straight line and a dashed line indicates a bond that may be a single covalent bond or alternatively a double covalent bond. But in the case where an atom's maximum valence would be exceeded by forming a double covalent bond, then the bond would be a single covalent bond.

It should be understood many of the reagents and starting materials used in the Schemes presented herein are readily available from various commercial suppliers, such as Sigma-Aldrich, Alfa Aesar, Tokyo Chemical Industry Co., LTD, etc. Moreover, many of these same reagents and starting materials can be modified to incorporate additional functional groups by using standard organic synthesis reactions.

### Small Molecule Modulators

The present invention relates to small molecule modulators of gp130. The present invention also relates to pharmaceutical compositions comprising at least one small molecule modulator of gp130. Also, disclosed herein is a topical formulation of small molecule modulators of a gp130 signaling cascade e.g., gp130, STAT-3, or NF-κB, methods of manufacturing of said formulation, and methods of use. In certain embodiments, the pharmaceutical composition comprises a topical formulation. In a non-limiting example, the novel topical formulations disclosed herein comprise gp130 signaling modulators (for example, a STAT-3 agonist) agonist that act to repair skin conditions.

In one aspect, small molecule modulators of the present invention comprise one or more compounds of the following Formula (I):

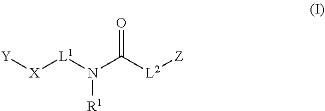

wherein:
X is optionally substituted cycloalkyl, optionally substituted heterocyclyl, optionally substituted heteroaryl, or optionally substituted aryl;
Y is optionally substituted cycloalkyl, optionally substituted heterocyclyl, optionally substituted heteroaryl, or optionally substituted aryl, or is absent;
Z is optionally substituted cycloalkyl, optionally substituted heterocyclyl, optionally substituted heteroaryl, or optionally substituted aryl or optionally substituted alkyl, optionally substituted alkenyl, optionally substituted alkynyl;
L¹ is optionally substituted alkyl, optionally substituted alkenyl, alkynyl, optionally substituted cycloalkyl, optionally substituted cycloalkenyl, alkylamino, aminoalkyl, alkylaminoalkyl, alkyl-O-alkyl, —O-alkyl, -alkyl-O—, —O—, —S—, or —N(R^{1a})—, or is absent;
L² is optionally substituted alkyl, optionally substituted alkenyl, alkynyl, optionally substituted cycloalkyl, optionally substituted cycloalkenyl, alkylamino, aminoalkyl, alkylaminoalkyl, alkyl-O-alkyl, —O-alkyl, -alkyl-O—, —O—, —S—, or —N(R^{1a})—, or is absent;
R¹ is H or C₁-C₆ alkyl;
each R^{1a} is H or C₁-C₆ alkyl;
or
a pharmaceutically acceptable salt thereof.

In another embodiment, compounds of the disclosure are compounds having Formula (I), wherein X is

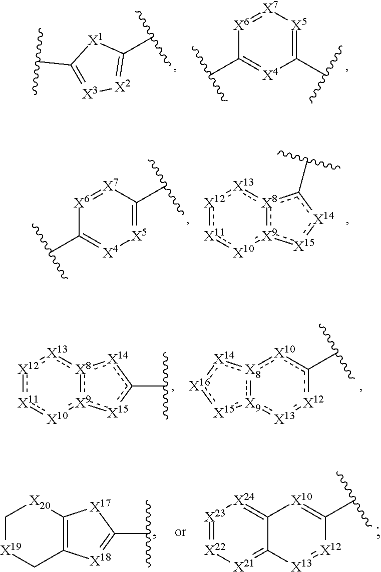

and Y is

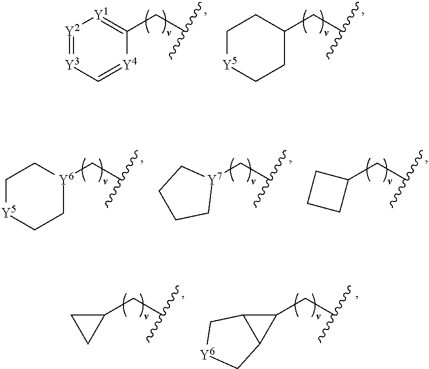

or is absent;
wherein,
   X¹ to X⁸ are each independently —C(R²)=, —N═, —O—, or —S—;
   X¹⁹ and X^{2′} are each independently —C(R²)(R³)—, —N(R⁴)—, —O—, or —S—;
   Y¹, Y², Y³ and Y⁴ are each independently —C(R²)=, or —N═;
   Y⁵ and Y⁸ are each independently —C(R²)(R³)—, —N(R⁴)—, —O—, or —S—;
   Y⁶ and Y⁷ are each independently —C(R²)—, or N;
   R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, amino(C₁-C₃)alkyl, (C₁-C₃)alkylamino, (C₁-C₃)alkoxyl,

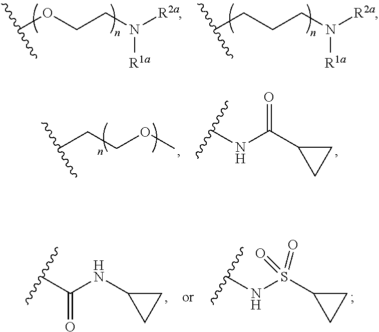

R⁴ is H, (C₁-C₃) alkyl, (C₁-C₃)haloalkyl, (C₁-C₃)alkenyl, (C₁-C₃)alkynyl, (C₁-C₃)alkoxyl,

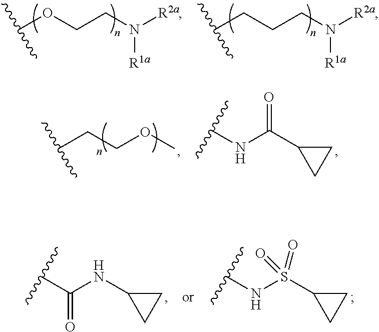

R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl;
v is 0, 1 or 2; and
n is 0, 1, 2, 3, 4, or 5.

In another embodiment, compounds of the disclosure are compounds having Formula (I), wherein Z is

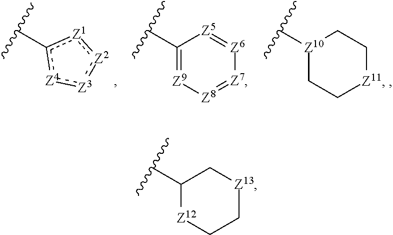

or is absent;
Z¹ to Z⁹ are each independently —C(R²)=, —N═, —O—, or —S—;
Z¹⁰ is —C(R²)— or N;
Z¹¹, Z¹² and Z¹³ are each independently —C(R²)(R³)—, —N(R⁴)—, —O—, or —S—;
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl,

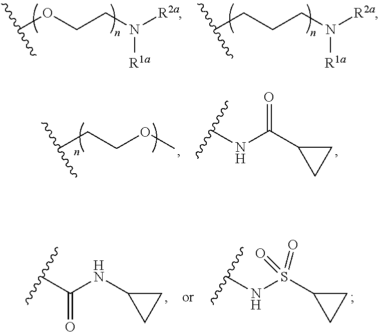

R⁴ is H, (C₁-C₃) alkyl, (C₁-C₃)haloalkyl, (C₁-C₃)alkenyl, (C₁-C₃)alkynyl, (C₁-C₃)alkoxyl,

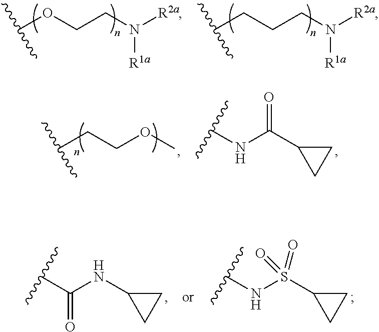

R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl; and
n is 0, 1, 2, 3, 4, or 5.

In an aspect, provided is a compound having a structure of Formula (X-I):

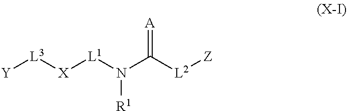

wherein:
A is O or S;
X is optionally substituted cycloalkyl, optionally substituted heterocyclyl, optionally substituted heteroaryl, or optionally substituted aryl;
Y is optionally substituted cycloalkyl, optionally substituted heterocyclyl, optionally substituted heteroaryl, or optionally substituted aryl, or is absent;
Z is optionally substituted cycloalkyl, optionally substituted heterocyclyl, optionally substituted heteroaryl, or optionally substituted aryl or optionally substituted alkyl, optionally substituted alkenyl, optionally substituted alkynyl;
L¹ is optionally substituted alkyl, optionally substituted alkenyl, alkynyl, optionally substituted cycloalkyl, optionally substituted cycloalkenyl, —O—, —S—, or —N(R^{1a})—, or is absent;
L² is optionally substituted alkyl, optionally substituted alkenyl, alkynyl, optionally substituted cycloalkyl, optionally substituted cycloalkenyl, —O—, —S—, or —N(R^{1a})—, or is absent;
L³ is optionally substituted alkyl, optionally substituted alkenyl, alkynyl, optionally substituted cycloalkyl, optionally substituted cycloalkenyl, —C(O)—, NH—C(O)—, —C(O)—NH—, —O—, —S—, or —N(R^{1a})—, or is absent;
R¹ is H or C₁-C₆ alkyl;
each R^{1a} is H or C₁-C₆ alkyl;
or
   a pharmaceutically acceptable salt thereof.

In certain embodiments, in Formula (X-I), X suitably may be:

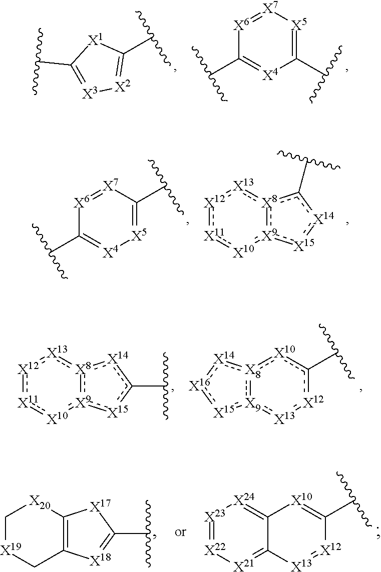

and Y is

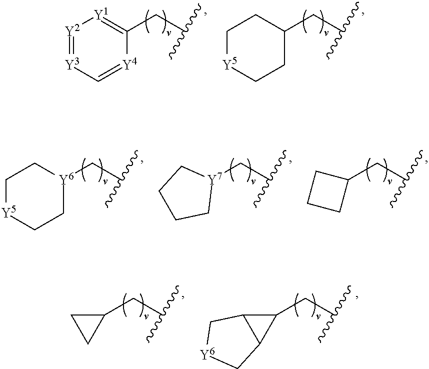

or is absent;
wherein,
   X¹ to X⁸ are each independently —C(R²)=, —N═, —O—, or —S—;
   X¹⁹ and X²⁰ are each independently —C(R²)(R³)—, —N(R⁴)—, —O—, or —S—;
   Y¹, Y², Y³ and Y⁴ are each independently —C(R²)=, or —N═;
   Y⁵ and Y⁸ are each independently —C(R²)(R³)—, —N(R⁴)—, —O—, or —S—;
   Y⁶ and Y⁷ are each independently —C(R²)—, or N;
   R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, amino(C₁-C₃)alkyl, (C₁-C₃)alkylamino, (C₁-C₃)alkoxyl, (C₃-C₆) branched alkyl, (C₃-C₆) branched haloalkyl, (C₃—C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

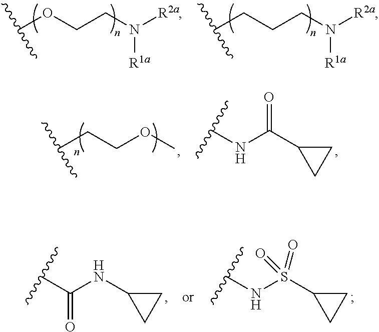

R⁴ is H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

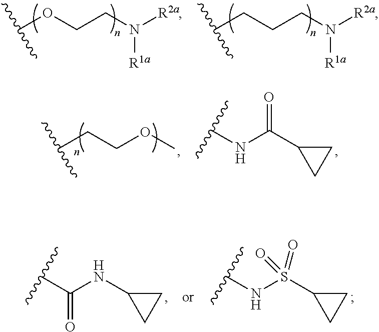

R^{1a} and R^{2a} are each independently H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, or —S(O)₂N(H)CH₃;
v is 0, 1 or 2; and
n is 0, 1, 2, 3, 4, or 5.

In certain embodiments, in Formula (X-I), Z may be:

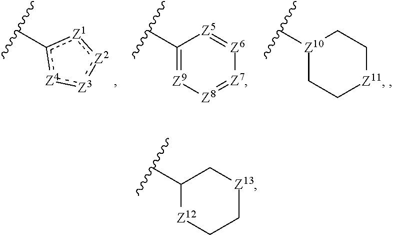

or is absent;
Z¹ to Z⁹ are each independently —C(R²)=, —N═, —O—, or —S—;
Z¹⁰ is —C(R²)— or N;
Z¹¹, Z¹² and Z¹³ are each independently —C(R²)(R³)—, —N(R⁴)—, —O—, or —S—;
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

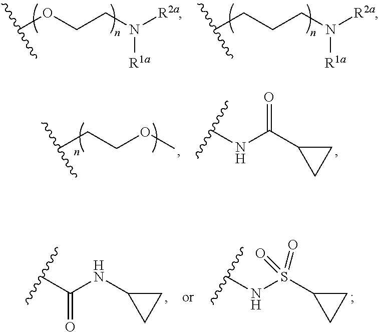

R⁴ is H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, —S(O)₂N(H)CH₃,

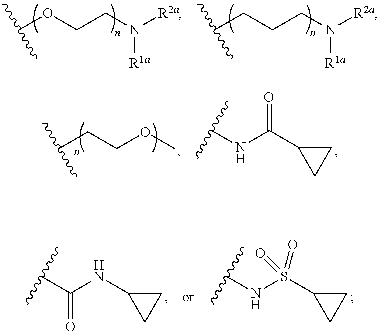

R^{1a} and R^{2a} are each independently H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, —S(O)₂N(H)(C₁-C₃)alkyl, or —S(O)₂N(H)CH₃; and
n is 0, 1, 2, 3, 4, or 5.

In another aspect, a compound of Formula (X-I) may have a structure of the following Formula (X-I-A):

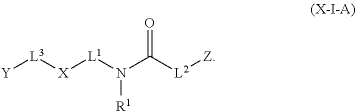

X, Y, Z, L¹, L², L³, and R¹ are as described above.

In another aspect, a compound of Formula (X-I) may have a structure of the following Formula (X-I-B):

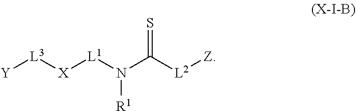

X, Y, Z, L¹, L², L³, and R¹ are as described above.

In certain aspect, L³ is selected from —C(O)—, NH—C(O)—, —C(O)—NH—, —O—, —S—, or —NH— or is absent. In certain aspect, L³ is absent. In certain aspect, L³ is —C(O)—.

In another embodiment, compounds of the disclosure are compounds having a structure of Formula (II):

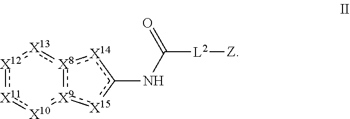

X⁸ to X¹⁵, L² and Z are as described above.

In another embodiment, compounds of the disclosure are compounds having a structure of Formula (IIb):

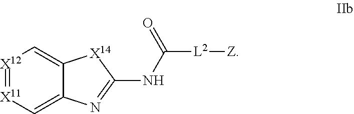

X¹¹, X¹², X¹⁴, L², and Z are as described above.

In another embodiment, compounds of the disclosure are compounds having a structure of Formula (IIc):

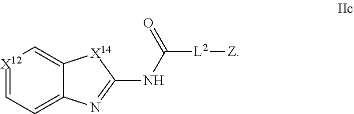

X¹², X¹⁴, L², and Z are as described above.

In another embodiment, compounds of the disclosure are compounds having a structure of Formula (IIa):

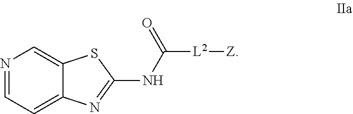

L², and Z are as described above.

In another embodiment, compounds of the disclosure are compounds having a structure of Formula (III):

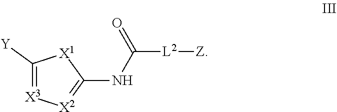

In another embodiment, compounds of the disclosure are compounds having a structure of Formula (IIIb):

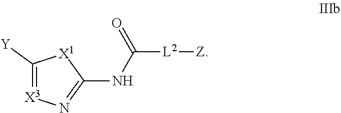

In another embodiment, compounds of the disclosure are compounds having a structure of Formula (IIIc):

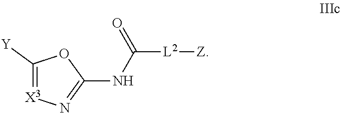

In another embodiment, compounds of the disclosure are compounds having a structure of Formula (IIIa):

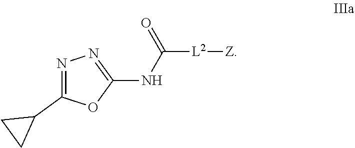

In another embodiment, compounds of the disclosure are compounds having a structure of Formula (IV):

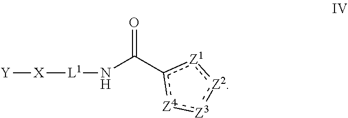

In another embodiment, compounds of the disclosure are compounds having a structure of Formula (IVb):

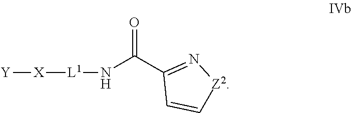

In another embodiment, compounds of the disclosure are compounds having a structure of Formula (IVa):

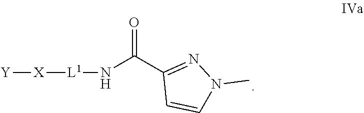

In another embodiment, compounds of the disclosure are compounds having a structure of Formula (V):

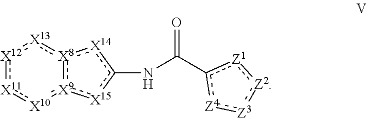

In another embodiment, compounds of the disclosure are compounds having a structure selected from the group consisting of Formula (Va), (Vb), (Vc), (Vd), (Ve), (Vf), (Vg), (Vh), (Vi), and (Vj):

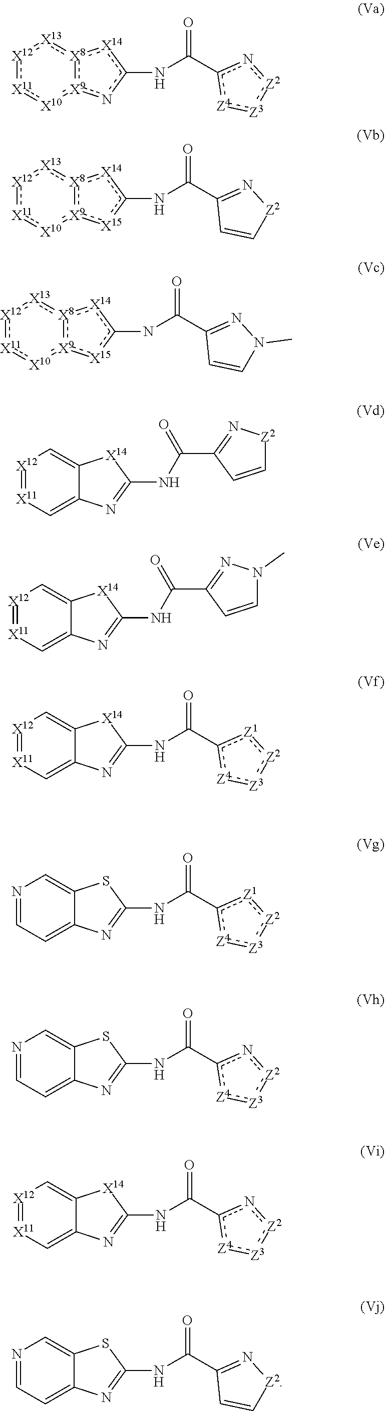

In another embodiment, compounds of the disclosure are compounds having a structure of Formula (VI):

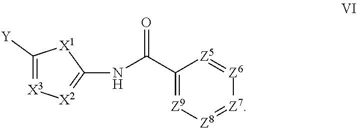

In another embodiment, compounds of the disclosure are compounds having a structure selected from the group consisting of Formula (VIa), (VIb), (VIc), (VId), (VIe), and (VIf):

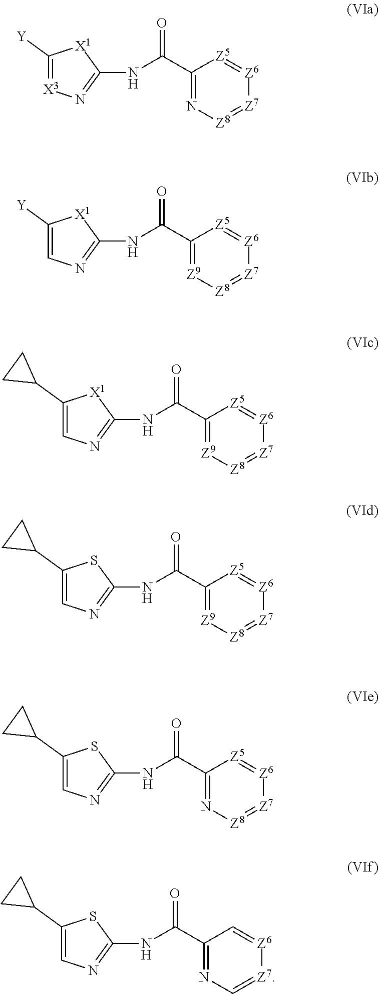

In another embodiment, compounds of the disclosure are compounds having a structure of Formula (VII):

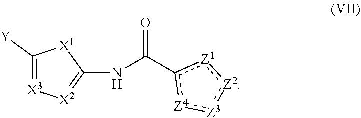

In another embodiment, compounds of the disclosure are compounds having a structure of Formula (VIII):

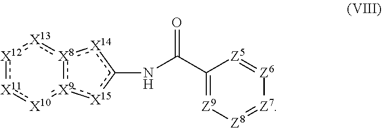

In another embodiment, compounds of the disclosure are compounds having a structure of the disclosure wherein L¹ is absent.

In another embodiment, compounds of the disclosure are compounds having a structure of the disclosure wherein L¹ is absent.

In another embodiment, compounds of the disclosure are compounds having a structure of the disclosure wherein L¹ is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —N(R^{1a})—(C₁-C₃)alkyl-, —(C₁-C₃)alkyl-N(R^{1a})—, —(C₁-C₃)alkyl-N(R^{1a})—(C₁-C₃)alkyl-, —O—(C₁-C₃)alkyl-, —(C₁-C₃)alkyl-O—, —N(R^{1a})—C(O)—, —C(O)—N(R^{1a})—, —N(R^{1a})—C(O)—N(R^{1a})—, —O—C(O)—N(R^{1a})—, —N(R^{1a})—C(O)—O—, —O—, —S—, or —N(R^{1a})—.

In another embodiment, compounds of the disclosure are compounds having a structure of the disclosure wherein L² is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —N(R^{1a})—(C₁-C₃)alkyl-, —(C₁-C₃)alkyl-N(R^{1a})—, —(C₁-C₃)alkyl-N(R^{1a})—(C₁-C₃)alkyl-, —O—(C₁-C₃)alkyl-, —(C₁-C₃)alkyl-O—, —N(R^{1a})—C(O)—, —C(O)—N(R^{1a})—, —N(R^{1a})—C(O)—N(R^{1a})—, —O—C(O)—N(R^{1a})—, —N(R^{1a})—C(O)—O—, —O—, —S—, or —N(R^{1a})—.

In a further embodiments, compounds of the above formulae are provided wherein L¹ is selected from the group consisting of:

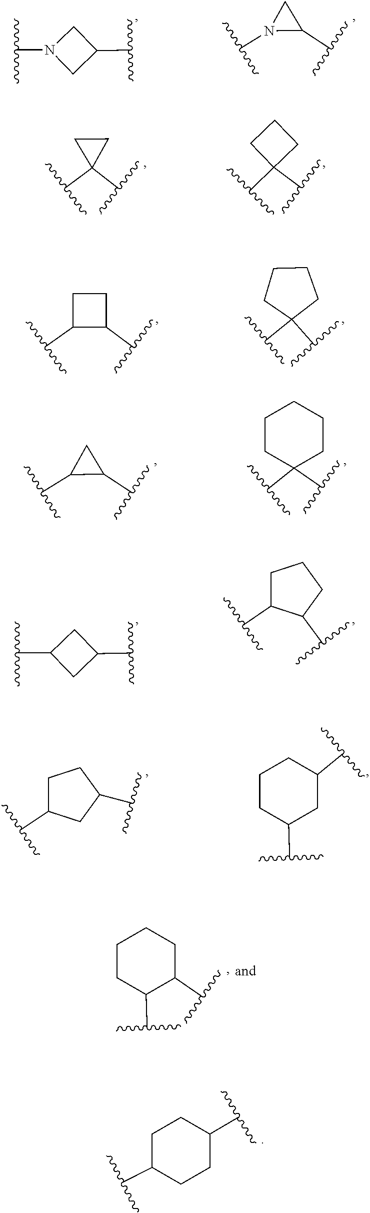

In a further embodiments, compounds of the above formulae are provided wherein L² is selected from the group consisting of:

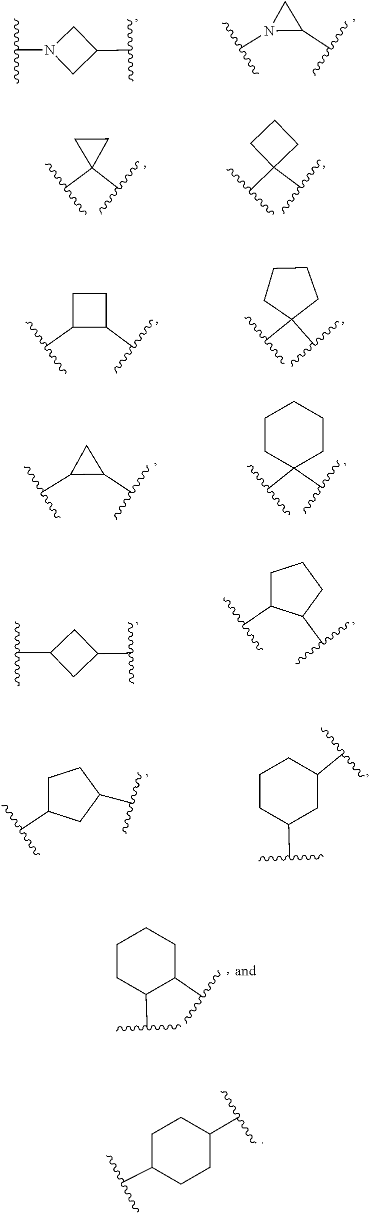

In another embodiment, compounds of the disclosure are compounds having Formula I, wherein: X is selected from the group consisting of:

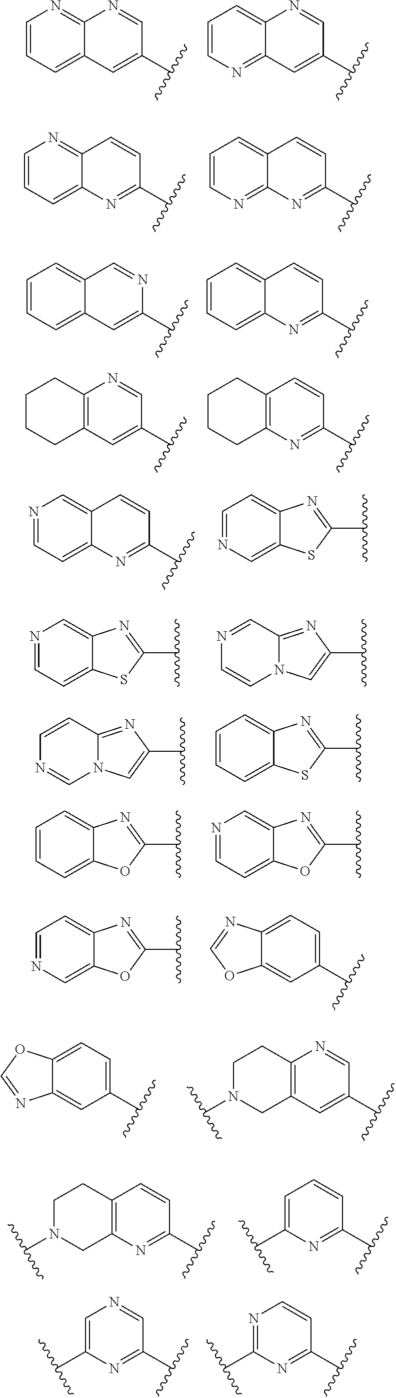

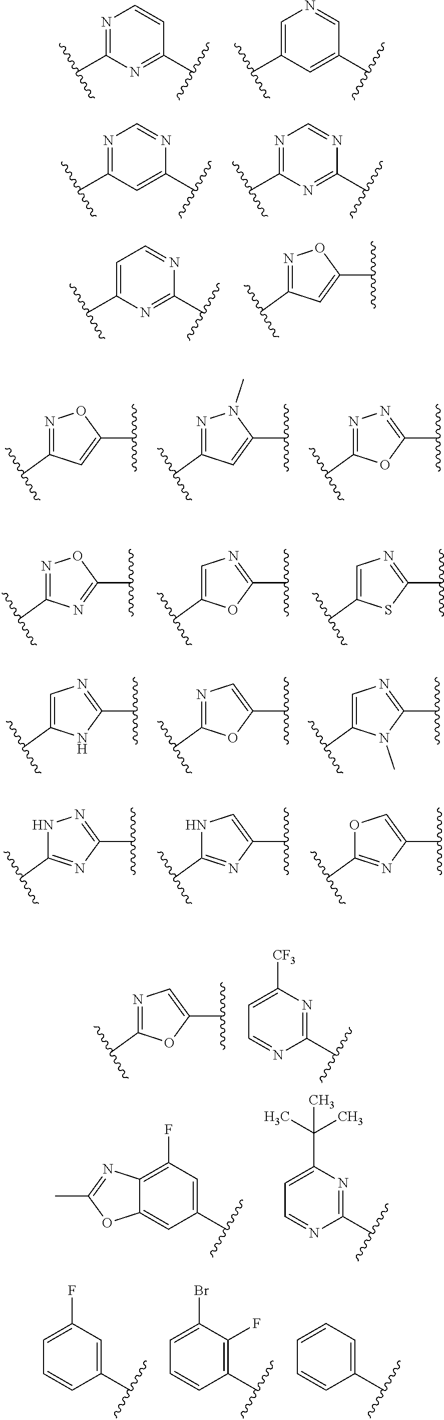

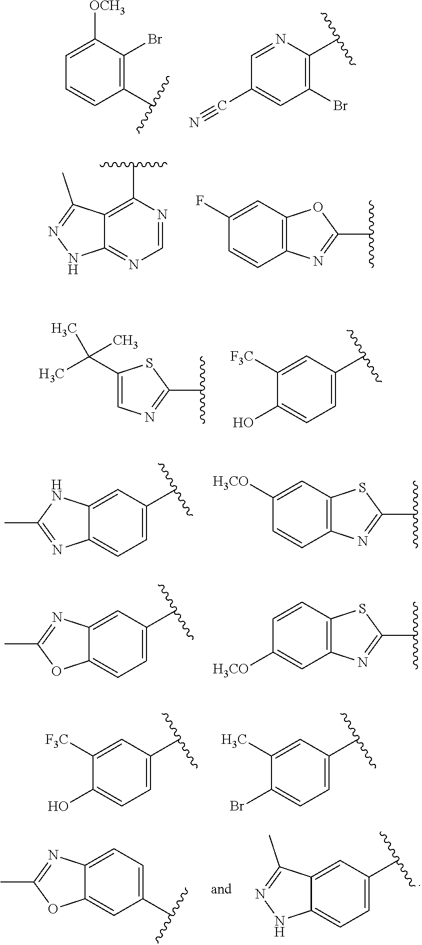

In another embodiment, compounds of the disclosure are compounds having Formula I, wherein: Z is selected from the group consisting of:

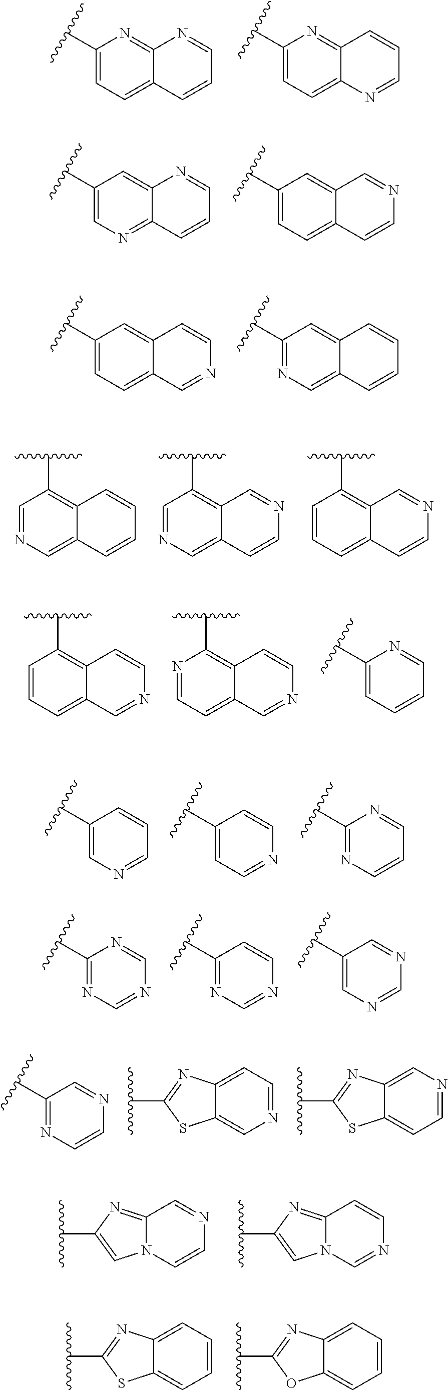

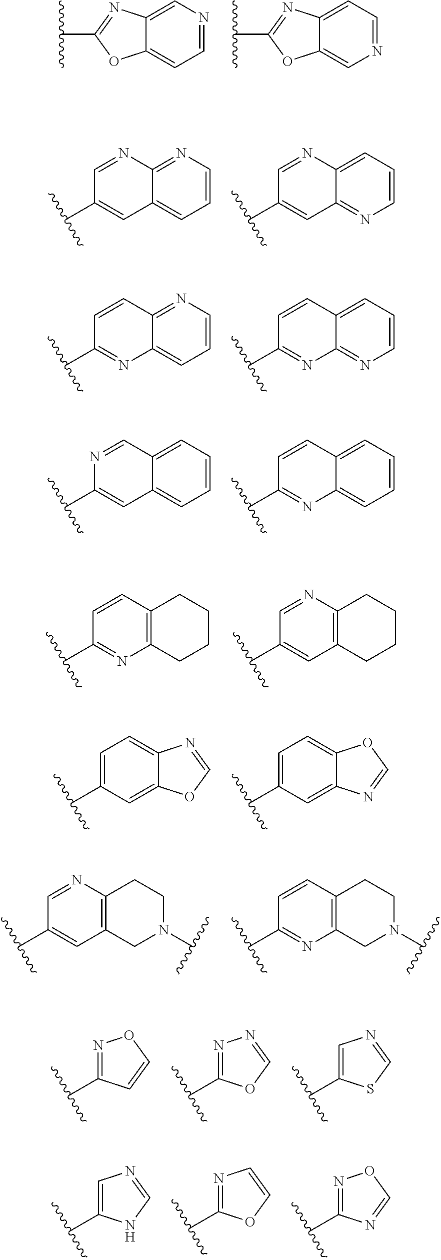

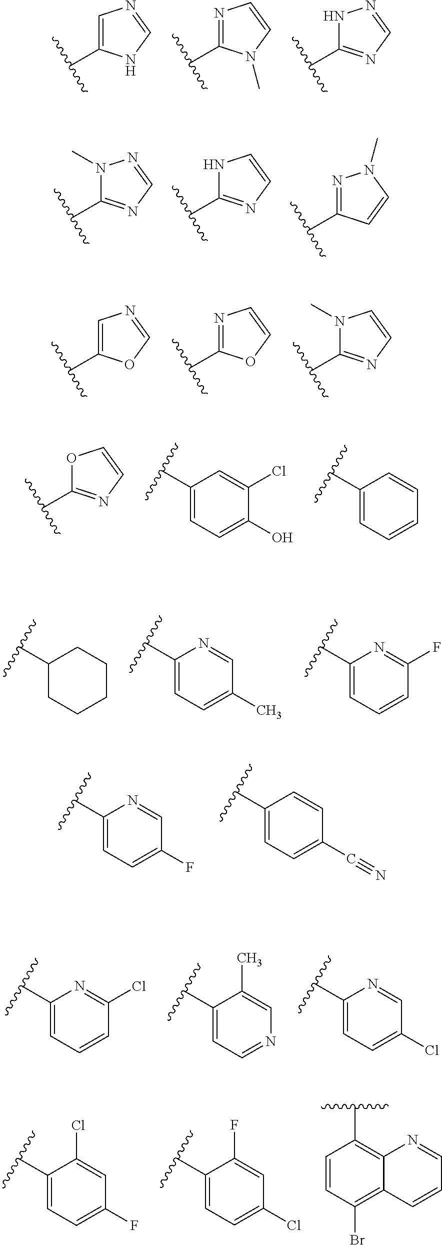

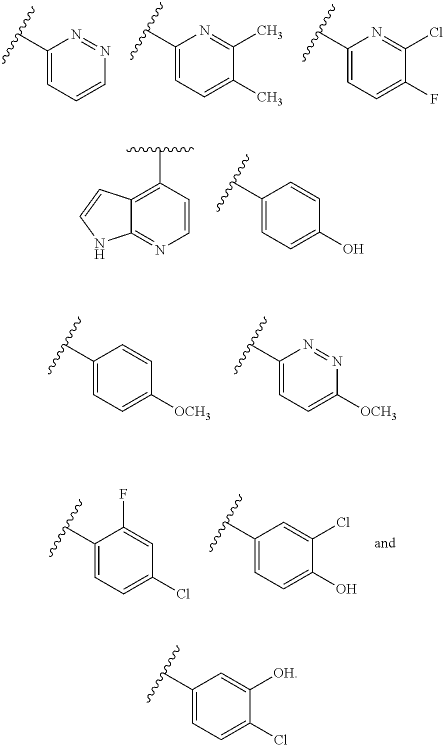

Exemplary compounds for use in the present compositions and formulations include the following shown in Table 1 and Table 2.

TABLE 1
Novel modulators of gp130 signaling

| Compound | Structure |
|---|---|
| 1 | 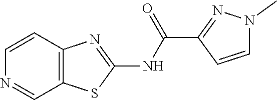 [SMILES: Cn1ccc(C(=O)Nc2nc3ccncc3s2)n1] |
| 2 | 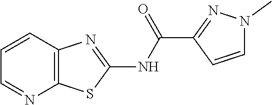 [SMILES: Cn1ccc(C(=O)Nc2nc3cccnc3s2)n1] |
| 3 | 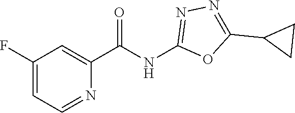 [SMILES: O=C(Nc1nnc(C2CC2)o1)c1cc(F)ccn1] |
| 4 | 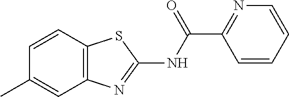 [SMILES: Cc1ccc2sc(NC(=O)c3ccccn3)nc2c1] |
| 5 | 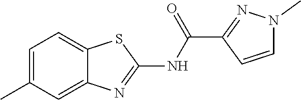 |
| 6 | 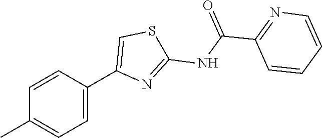 [SMILES: Cc1ccc(-c2csc(NC(=O)c3ccccn3)n2)cc1] |
| 7 | 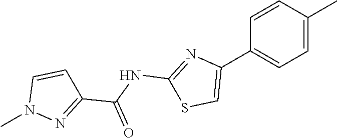 |
| 8 | 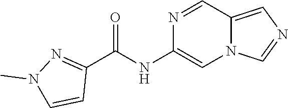 [SMILES: Cn1ccc(C(=O)Nc2cn3cncc3cn2)n1] |
| 9 | 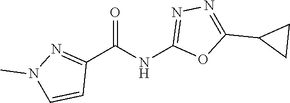 [SMILES: Cn1ccc(C(=O)Nc2nnc(C3CC3)o2)n1] |
| 10 | 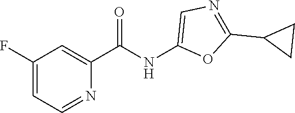 [SMILES: O=C(Nc1cnc(C2CC2)o1)c1cc(F)ccn1] |
| 11 | 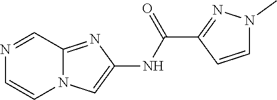 [SMILES: Cn1ccc(C(=O)Nc2cn3ccncc3n2)n1] |
| 12 | 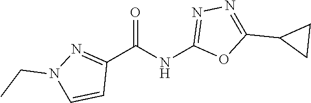 [SMILES: CCn1ccc(C(=O)Nc2nnc(C3CC3)o2)n1] |
| 13 | 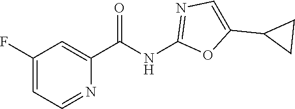 [SMILES: O=C(Nc1ncc(C2CC2)o1)c1cc(F)ccn1] |
| 14 | 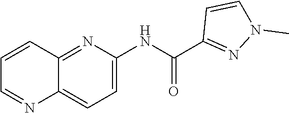 [SMILES: Cn1ccc(C(=O)Nc2ccc3ncccc3n2)n1] |
| 15 | 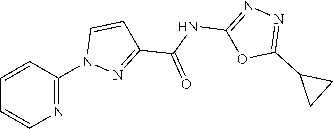 [SMILES: O=C(Nc1nnc(C2CC2)o1)c1ccn(-c2ccccn2)n1] |
| 16 | 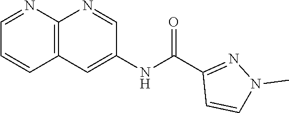 [SMILES: Cn1ccc(C(=O)Nc2cnc3ncccc3c2)n1] |
| 17 | 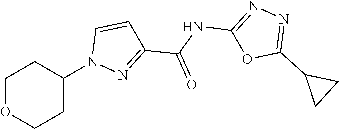 [SMILES: O=C(Nc1nnc(C2CC2)o1)c1ccn(C2CCOCC2)n1] |
| 18 | 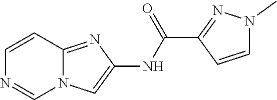 [SMILES: Cn1ccc(C(=O)Nc2cn3cnccc3n2)n1] |
| 19 | 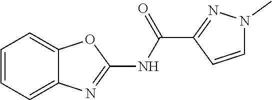 [SMILES: Cn1ccc(C(=O)Nc2nc3ccccc3o2)n1] |
| 20 | 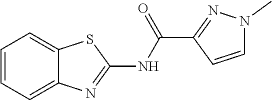 [SMILES: Cn1ccc(C(=O)Nc2nc3ccccc3s2)n1] |
| 21 | 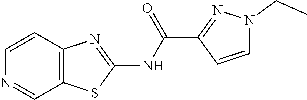 [SMILES: CCn1ccc(C(=O)Nc2nc3ccncc3s2)n1] |
| 22 | 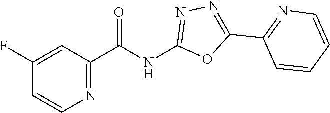 [SMILES: O=C(Nc1nnc(-c2ccccn2)o1)c1cc(F)ccn1] |
| 23 | 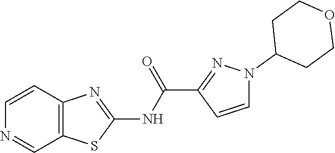 [SMILES: O=C(Nc1nc2ccncc2s1)c1ccn(C2CCOCC2)n1] |
| 24 | 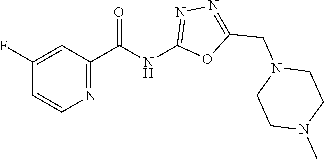 [SMILES: CN1CCN(Cc2nnc(NC(=O)c3cc(F)ccn3)o2)CC1] |
| 25 | 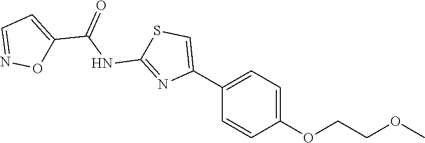 [SMILES: COCCOc1ccc(-c2csc(NC(=O)c3ccno3)n2)cc1] |
| 26 | 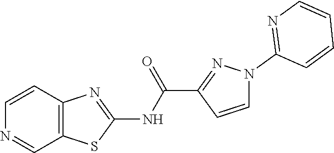 [SMILES: O=C(Nc1nc2ccncc2s1)c1ccn(-c2ccccn2)n1] |
| 27 | 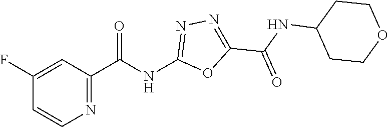 [SMILES: O=C(Nc1nnc(C(=O)NC2CCOCC2)o1)c1cc(F)ccn1] |
| 28 | 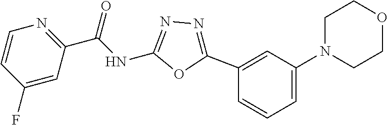 [SMILES: O=C(Nc1nnc(-c2cccc(N3CCOCC3)c2)o1)c1cc(F)ccn1] |
| 29 | 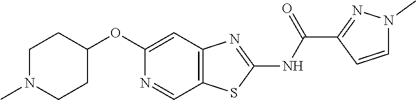 |
| 30 | 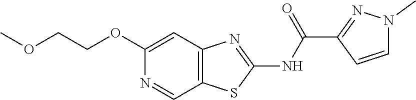 |
| 31 | 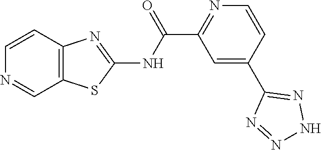 [SMILES: O=C(Nc1nc2ccncc2s1)c1cc(-c2nn[nH]n2)ccn1] |
| 32 | 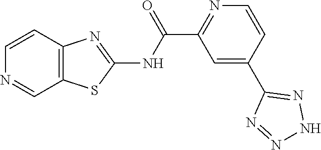 [SMILES: O=C(Nc1nc2ccncc2s1)c1cc(-c2nn[nH]n2)ccn1] |
| 33 | 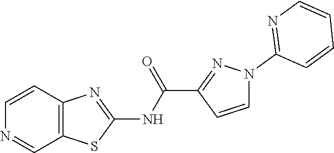 [SMILES: O=C(Nc1nc2ccncc2s1)c1ccn(-c2ccccn2)n1] |
| 34 | 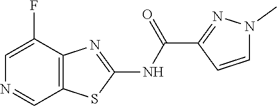 [SMILES: Cn1ccc(C(=O)Nc2nc3c(F)cncc3s2)n1] |
| 35 | 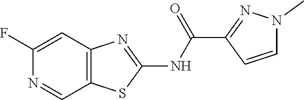 [SMILES: Cn1ccc(C(=O)Nc2nc3cc(F)ncc3s2)n1] |
| 36 | 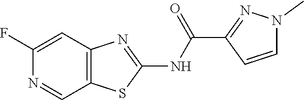 [SMILES: Cn1ccc(C(=O)Nc2nc3cc(F)ncc3s2)n1] |
| 37 | 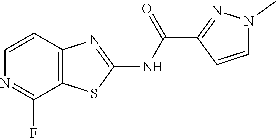 [SMILES: Cn1ccc(C(=O)Nc2nc3ccnc(F)c3s2)n1] |
| 38 | 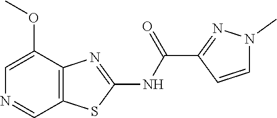 [SMILES: COc1cncc2sc(NC(=O)c3ccn(C)n3)nc12] |
| 39 | 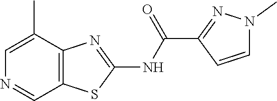 [SMILES: Cc1cncc2sc(NC(=O)c3ccn(C)n3)nc12] |
| 40 | 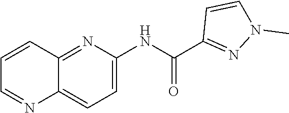 [SMILES: Cn1ccc(C(=O)Nc2ccc3ncccc3n2)n1] |
| 41 | 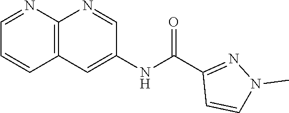 [SMILES: Cn1ccc(C(=O)Nc2cnc3ncccc3c2)n1] |
| 42 | 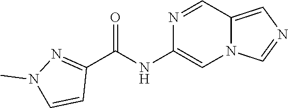 [SMILES: Cn1ccc(C(=O)Nc2cn3cncc3cn2)n1] |
| 43 | 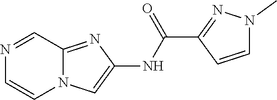 [SMILES: Cn1ccc(C(=O)Nc2cn3ccncc3n2)n1] |
| 44 | 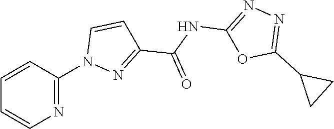 [SMILES: O=C(Nc1nnc(C2CC2)o1)c1ccn(-c2ccccn2)n1] |
| 45 | 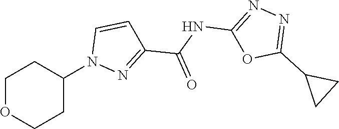 [SMILES: O=C(Nc1nnc(C2CC2)o1)c1ccn(C2CCOCC2)n1] |
| 46 | 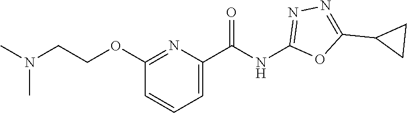 [SMILES: CN(C)CCOc1cccc(C(=O)Nc2nnc(C3CC3)o2)n1] |
| 47 | 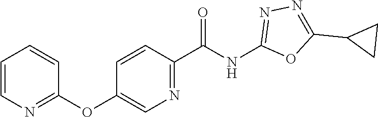 [SMILES: O=C(Nc1nnc(C2CC2)o1)c1ccc(Oc2ccccn2)cn1] |
| 48 | 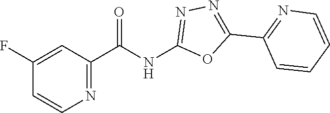 [SMILES: O=C(Nc1nnc(-c2ccccn2)o1)c1cc(F)ccn1] |
| 49 | 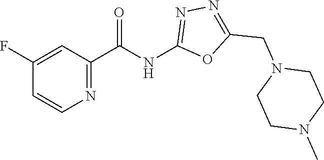 [SMILES: CN1CCN(Cc2nnc(NC(=O)c3cc(F)ccn3)o2)CC1] |
| 50 | 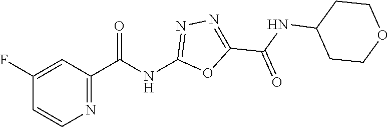 [SMILES: O=C(Nc1nnc(C(=O)NC2CCOCC2)o1)c1cc(F)ccn1] |
| 51 | 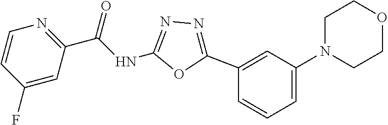 [SMILES: O=C(Nc1nnc(-c2cccc(N3CCOCC3)c2)o1)c1cc(F)ccn1] |
| 52 | 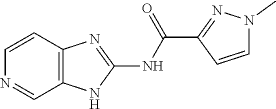 [SMILES: Cn1ccc(C(=O)Nc2nc3ccncc3[nH]2)n1] |
| 53 | 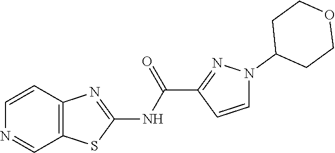 [SMILES: O=C(Nc1nc2ccncc2s1)c1ccn(C2CCOCC2)n1] |
| 54 | 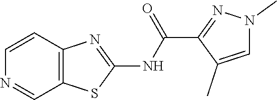 [SMILES: Cc1cn(C)nc1C(=O)Nc1nc2ccncc2s1] |
| 55 | 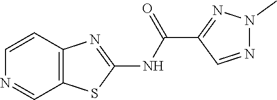 [SMILES: Cn1ncc(C(=O)Nc2nc3ccncc3s2)n1] |
| 56 | 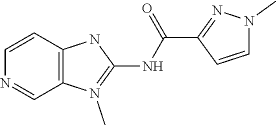 |
| 57 | 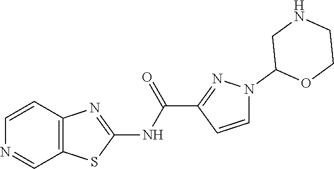 [SMILES: O=C(Nc1nc2ccncc2s1)c1ccn(C2CNCCO2)n1] |
| 58 | 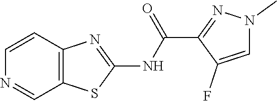 [SMILES: Cn1cc(F)c(C(=O)Nc2nc3ccncc3s2)n1] |
| 59 | 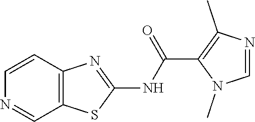 [SMILES: Cc1ncn(C)c1C(=O)Nc1nc2ccncc2s1] |
| 60 | 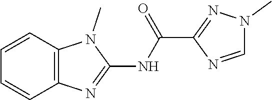 [SMILES: Cn1cnc(C(=O)Nc2nc3ccccc3n2C)n1] |
| 61 | 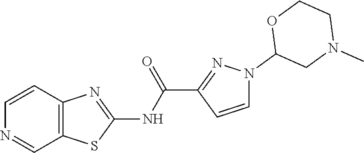 [SMILES: CN1CCOC(n2ccc(C(=O)Nc3nc4ccncc4s3)n2)C1] |
| 62 | 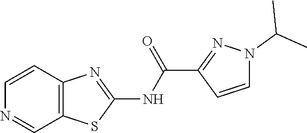 [SMILES: CC(C)n1ccc(C(=O)Nc2nc3ccncc3s2)n1] |
| 63 | 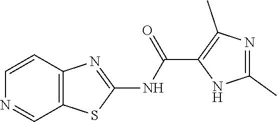 [SMILES: Cc1nc(C)c(C(=O)Nc2nc3ccncc3s2)[nH]1] |
| 64 | 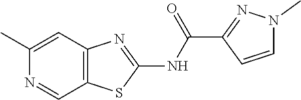 |
| 65 | 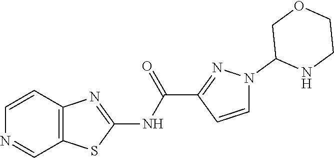 [SMILES: O=C(Nc1nc2ccncc2s1)c1ccn(C2COCCN2)n1] |
| 66 | 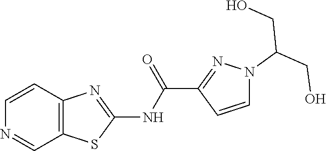 [SMILES: O=C(Nc1nc2ccncc2s1)c1ccn(C(CO)CO)n1] |
| 67 | 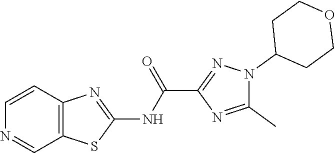 [SMILES: Cc1nc(C(=O)Nc2nc3ccncc3s2)nn1C1CCOCC1] |
| 68 | 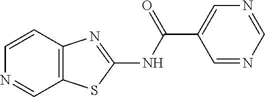 [SMILES: O=C(Nc1nc2ccncc2s1)c1cncnc1] |
| 69 | 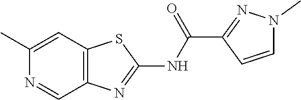 |
| 70 | 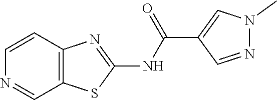 [SMILES: Cn1cc(C(=O)Nc2nc3ccncc3s2)cn1] |
| 71 | 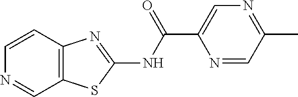 [SMILES: Cc1cnc(C(=O)Nc2nc3ccncc3s2)cn1] |
| 72 | 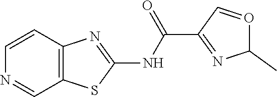 |
| 73 | 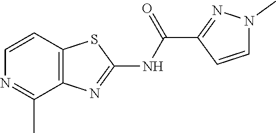 [SMILES: Cc1nccc2sc(NC(=O)c3ccn(C)n3)nc12] |
| 74 | 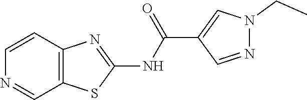 [SMILES: CCn1cc(C(=O)Nc2nc3ccncc3s2)cn1] |
| 75 | 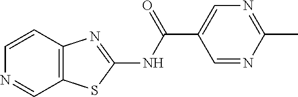 [SMILES: Cc1ncc(C(=O)Nc2nc3ccncc3s2)cn1] |
| 76 | 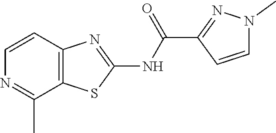 [SMILES: Cc1nccc2nc(NC(=O)c3ccn(C)n3)sc12] |
| 77 | 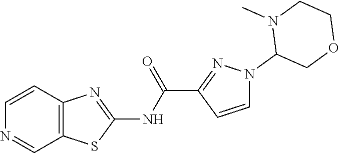 [SMILES: CN1CCOCC1n1ccc(C(=O)Nc2nc3ccncc3s2)n1] |
| 78 | 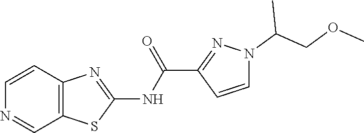 [SMILES: COCC(C)n1ccc(C(=O)Nc2nc3ccncc3s2)n1] |
| 79 | 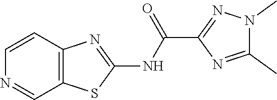 [SMILES: Cc1nc(C(=O)Nc2nc3ccncc3s2)nn1C] |
| 80 | 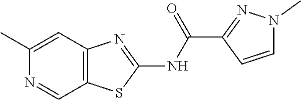 |
| 81 | 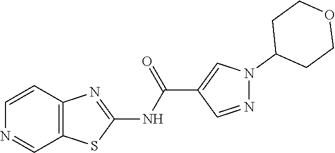 [SMILES: O=C(Nc1nc2ccncc2s1)c1cnn(C2CCOCC2)c1] |
| 82 | 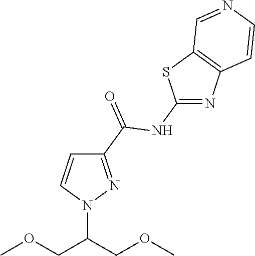 [SMILES: COCC(COC)n1ccc(C(=O)Nc2nc3ccncc3s2)n1] |
| 83 | 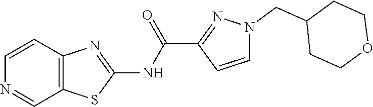 [SMILES: O=C(Nc1nc2ccncc2s1)c1ccn(CC2CCOCC2)n1] |
| 84 | 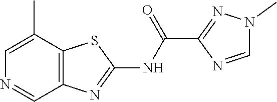 [SMILES: Cc1cncc2nc(NC(=O)c3ncn(C)n3)sc12] |
| 85 | 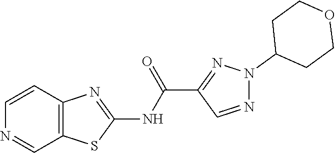 [SMILES: O=C(Nc1nc2ccncc2s1)c1cnn(C2CCOCC2)n1] |
| 86 | 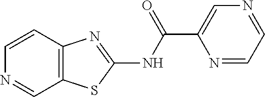 [SMILES: O=C(Nc1nc2ccncc2s1)c1cnccn1] |
| 87 | 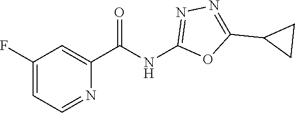 [SMILES: O=C(Nc1nnc(C2CC2)o1)c1cc(F)ccn1] |
| 88 | 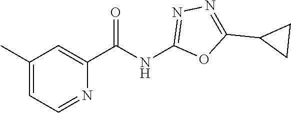 [SMILES: Cc1ccnc(C(=O)Nc2nnc(C3CC3)o2)c1] |
| 89 | 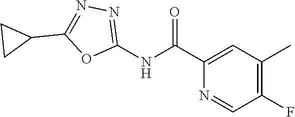 [SMILES: Cc1cc(C(=O)Nc2nnc(C3CC3)o2)ncc1F] |
| 90 | 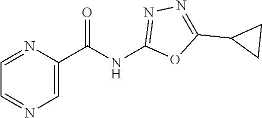 [SMILES: O=C(Nc1nnc(C2CC2)o1)c1cnccn1] |
| 91 | 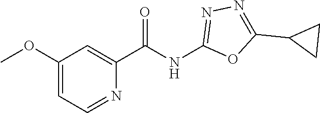 [SMILES: COc1ccnc(C(=O)Nc2nnc(C3CC3)o2)c1] |
| 92 | 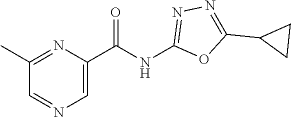 [SMILES: Cc1cncc(C(=O)Nc2nnc(C3CC3)o2)n1] |
| 93 | 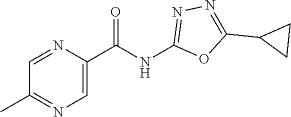 [SMILES: Cc1cnc(C(=O)Nc2nnc(C3CC3)o2)cn1] |
| 94 | 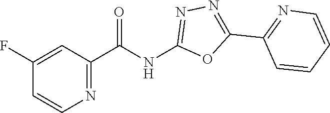 [SMILES: O=C(Nc1nnc(-c2ccccn2)o1)c1cc(F)ccn1] |
| 95 | 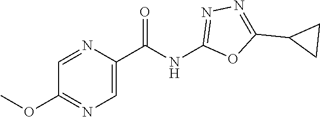 [SMILES: COc1cnc(C(=O)Nc2nnc(C3CC3)o2)cn1] |
| 96 | 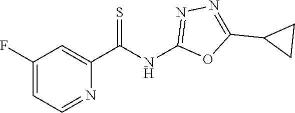 [SMILES: Fc1ccnc(C(=S)Nc2nnc(C3CC3)o2)c1] |
| 97 | 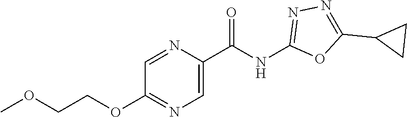 [SMILES: COCCOc1cnc(C(=O)Nc2nnc(C3CC3)o2)cn1] |
| 98 | 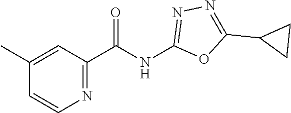 [SMILES: Cc1ccnc(C(=O)Nc2nnc(C3CC3)o2)c1] |
| 99 | 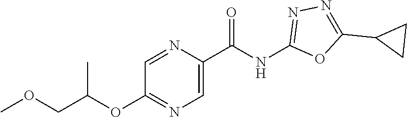 [SMILES: COCC(C)Oc1cnc(C(=O)Nc2nnc(C3CC3)o2)cn1] |
| 100 | 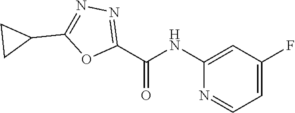 [SMILES: O=C(Nc1cc(F)ccn1)c1nnc(C2CC2)o1] |
| 101 | 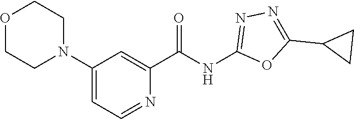 [SMILES: O=C(Nc1nnc(C2CC2)o1)c1cc(N2CCOCC2)ccn1] |
| 102 | 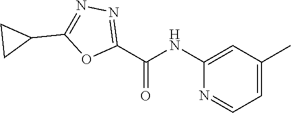 [SMILES: Cc1ccnc(NC(=O)c2nnc(C3CC3)o2)c1] |
| 103 | 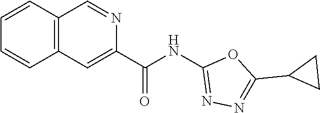 [SMILES: O=C(Nc1nnc(C2CC2)o1)c1cc2ccccc2cn1] |
| 104 | 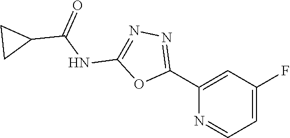 [SMILES: O=C(Nc1nnc(-c2cc(F)ccn2)o1)C1CC1] |
| 105 | 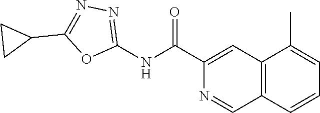 [SMILES: Cc1cccc2cnc(C(=O)Nc3nnc(C4CC4)o3)cc12] |
| 106 | 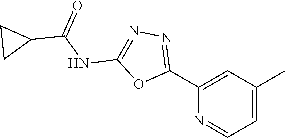 [SMILES: Cc1ccnc(-c2nnc(NC(=O)C3CC3)o2)c1] |
| 107 | 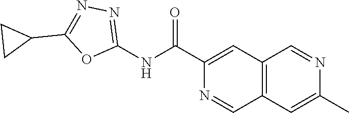 [SMILES: Cc1cc2cnc(C(=O)Nc3nnc(C4CC4)o3)cc2cn1] |
| 108 | 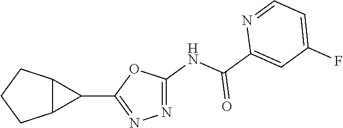 [SMILES: O=C(Nc1nnc(C2C3CCCC32)o1)c1cc(F)ccn1] |
| 109 | 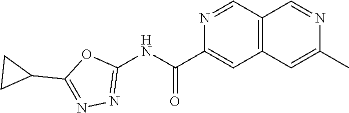 [SMILES: Cc1cc2cc(C(=O)Nc3nnc(C4CC4)o3)ncc2cn1] |
| 110 | 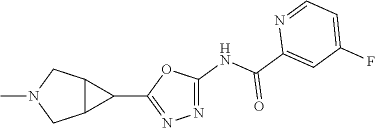 [SMILES: CN1CC2C(C1)C2c1nnc(NC(=O)c2cc(F)ccn2)o1] |
| 111 | 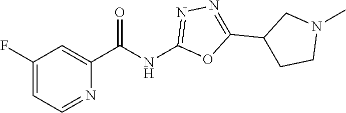 [SMILES: CN1CCC(c2nnc(NC(=O)c3cc(F)ccn3)o2)C1] |
| 112 | 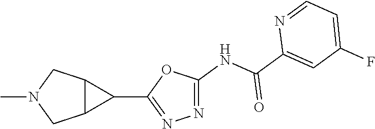 [SMILES: CN1CC2C(C1)C2c1nnc(NC(=O)c2cc(F)ccn2)o1] |
| 113 | 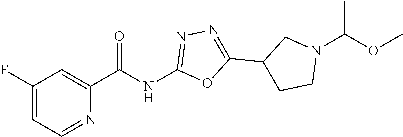 [SMILES: COC(C)N1CCC(c2nnc(NC(=O)c3cc(F)ccn3)o2)C1] |
| 114 | 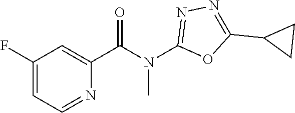 [SMILES: CN(C(=O)c1cc(F)ccn1)c1nnc(C2CC2)o1] |
| 115 | 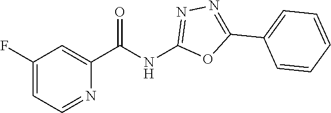 [SMILES: O=C(Nc1nnc(-c2ccccc2)o1)c1cc(F)ccn1] |
| 116 | 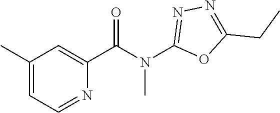 [SMILES: CCc1nnc(N(C)C(=O)c2cc(C)ccn2)o1] |
| 117 | 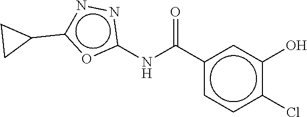 |
| 118 | 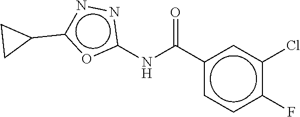 |
| 119 | 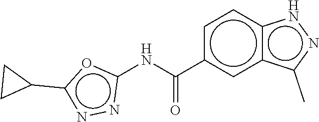 |
| 120 | 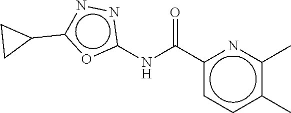 |
| 121 | 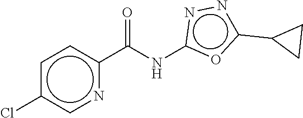 |
| 122 | 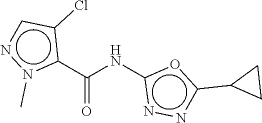 |
| 123 | 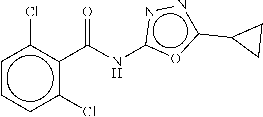 |
| 124 | 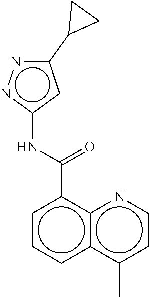 |
| 125 | 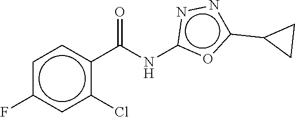 |
| 126 | 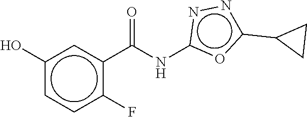 |
| 127 | 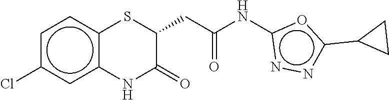 |
| 128 | 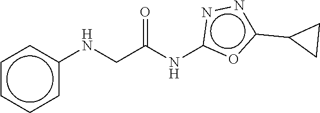 |
| 129 | 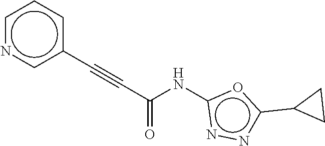 |
| 130 | 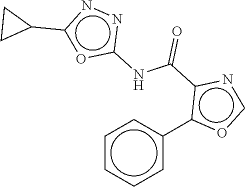 |
| 131 | 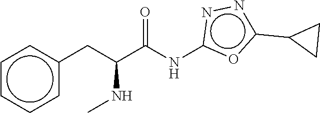 |
| 132 | 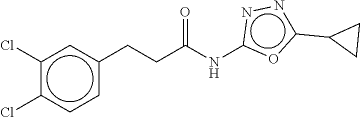 |
| 133 | 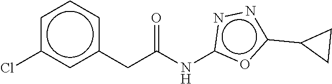 |
| 134 | 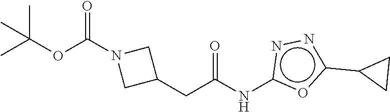 |
| 135 | 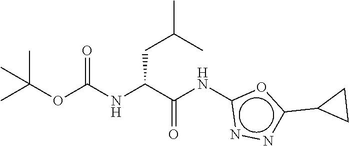 |
| 136 | 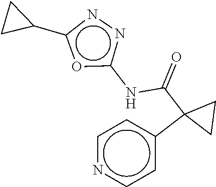 |
| 137 | 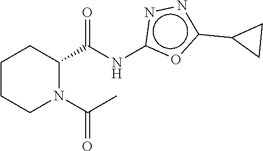 |
| 138 | 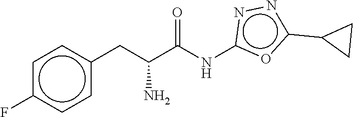 |
| 139 | 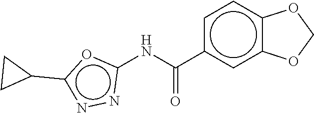 |
| 140 | 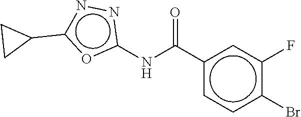 |
| 141 | 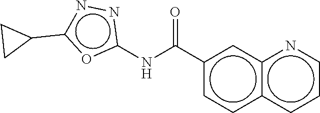 |
| 142 | 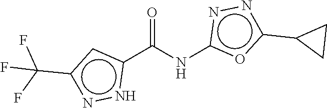 |
| 143 | 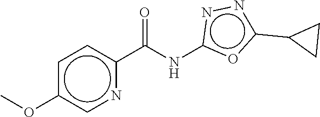 |
| 144 | 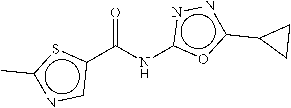 |
| 145 | 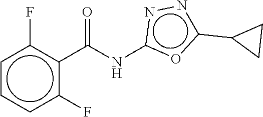 |
| 146 | 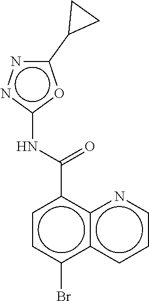 |
| 147 | 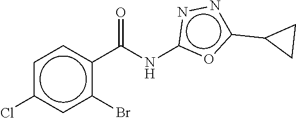 |
| 148 | 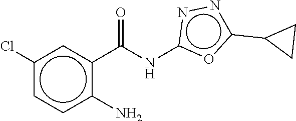 |
| 149 | 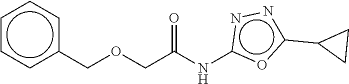 |
| 150 | 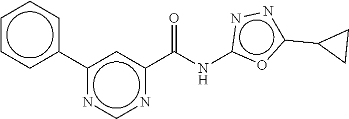 |
| 151 | 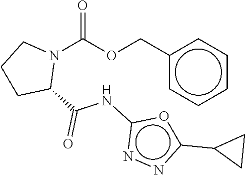 |
| 152 | 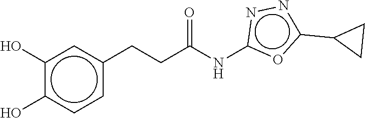 |
| 153 | 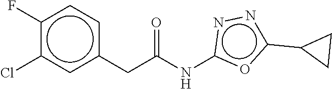 |
| 154 | 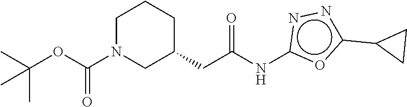 |
| 155 | 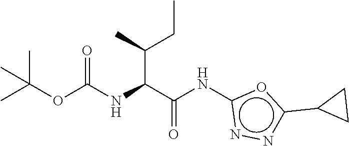 |
| 156 | 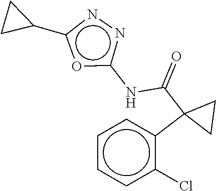 |
| 157 | 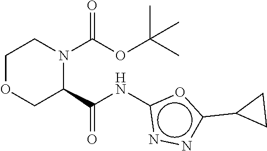 |
| 158 | 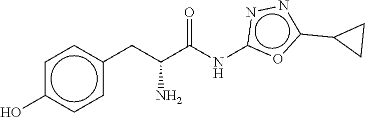 |
| 159 | 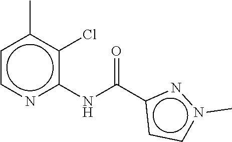 |
| 160 | 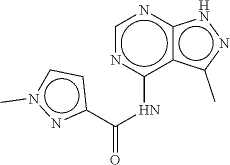 |
| 161 | 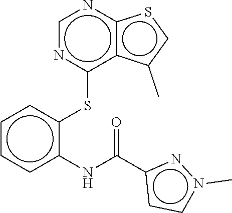 |
| 162 | 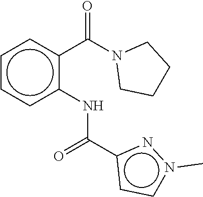 |
| 163 | 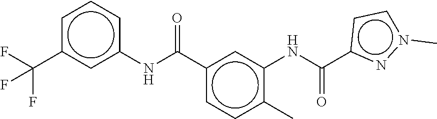 |
| 164 | 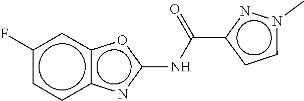 |
| 165 | 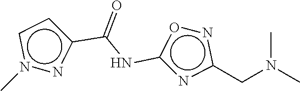 |
| 166 | 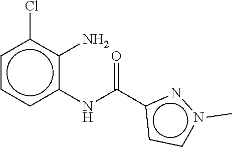 |
| 167 | 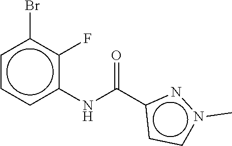 |
| 168 | 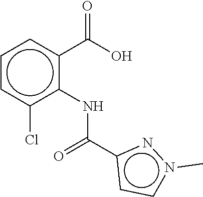 |
| 169 | 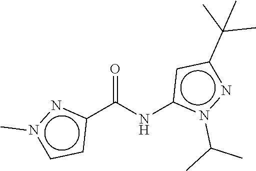 |
| 170 | 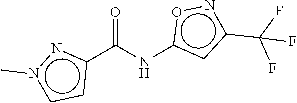 |
| 171 | 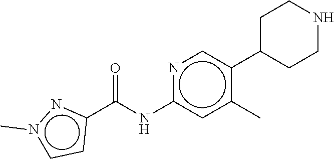 |
| 172 | 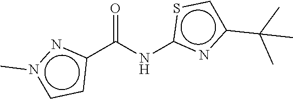 |
| 173 | 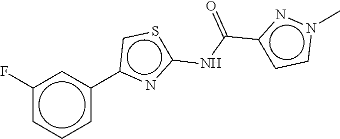 |
| 174 | 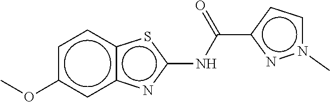 |
| 175 | 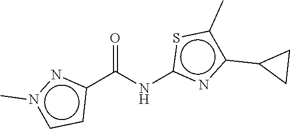 |
| 176 | 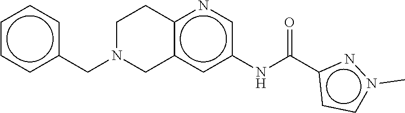 |
| 177 | 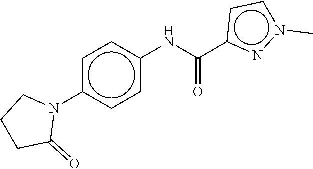 |
| 178 | 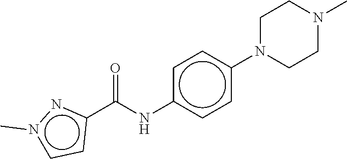 |
| 179 | 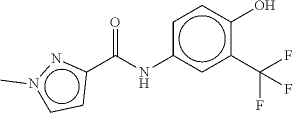 |
| 180 | 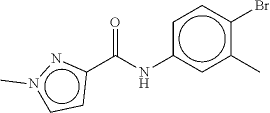 |
| 181 | 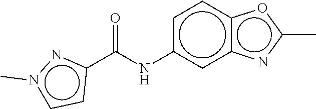 |
| 182 | 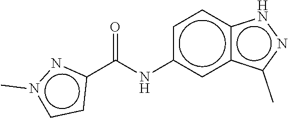 |
| 183 | 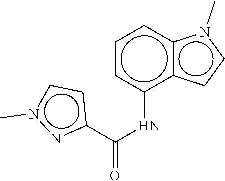 |
| 184 | 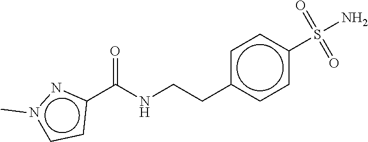 |
| 185 | 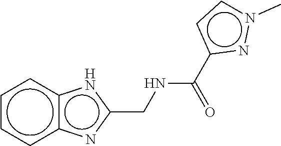 |
| 186 | 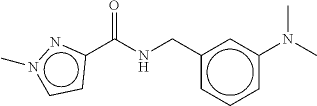 |
| 187 | 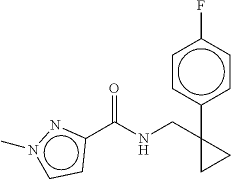 |
| 188 | 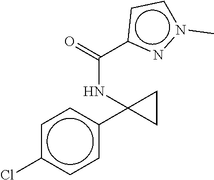 |
| 189 | 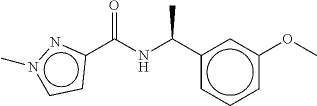 |
| 190 | 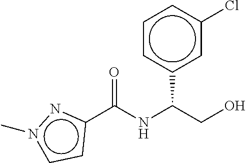 |
| 191 | 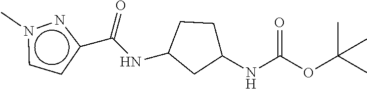 |
| 192 | 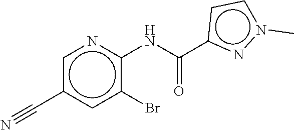 |
| 193 | 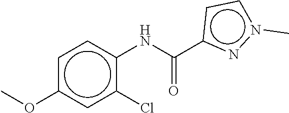 |
| 194 | 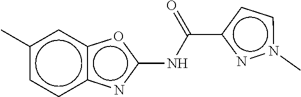 |
| 195 | 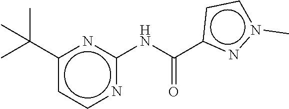 |
| 196 | 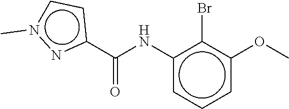 |
| 197 | 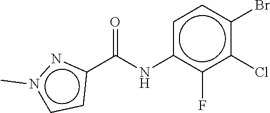 |
| 198 | 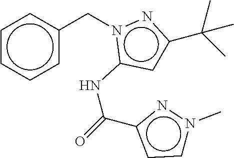 |
| 199 | 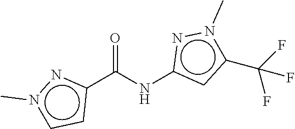 |
| 200 | 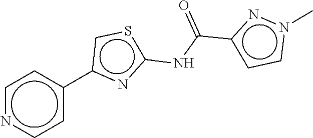 |
| 201 | 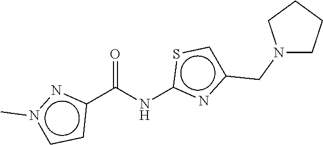 |
| 202 | 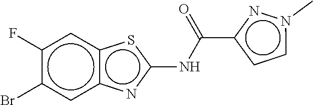 |
| 203 | 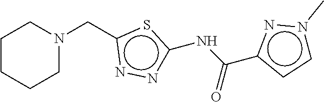 |
| 204 | 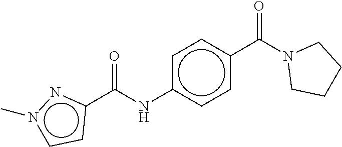 |
| 205 | 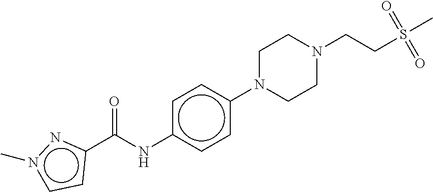 |
| 206 | 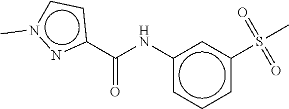 |
| 207 | 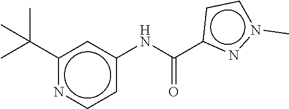 |
| 208 | 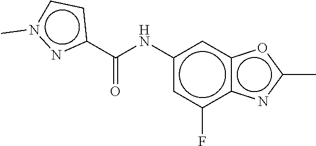 |
| 209 | 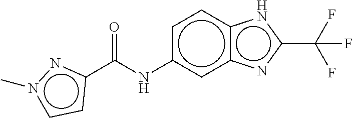 |
| 210 | 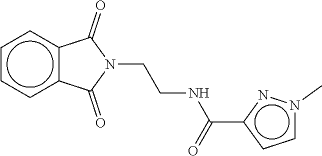 |
| 211 | 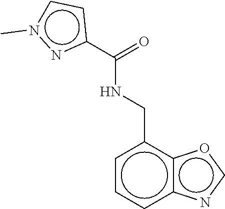 |
| 212 | 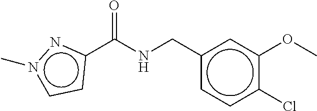 |
| 213 | 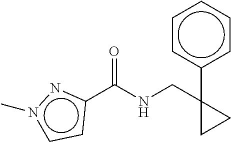 |
| 214 | 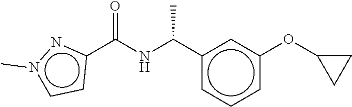 |
| 215 | 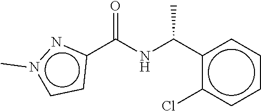 |
| 216 | 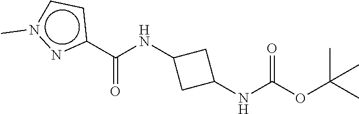 |
| 217 | 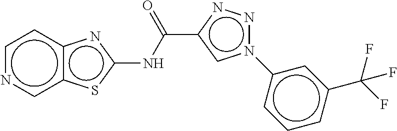 |
| 218 | 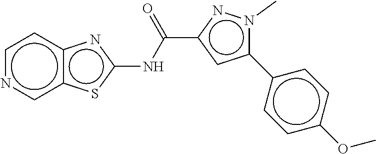 |
| 219 | 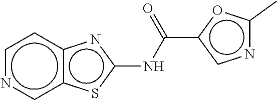 |
| 220 | 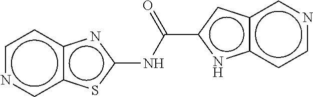 |
| 221 | 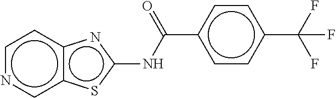 |
| 222 | 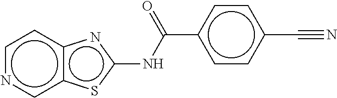 |
| 223 | 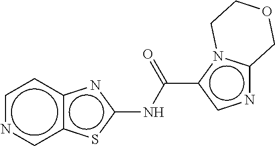 |
| 224 | 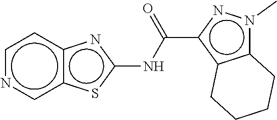 |
| 225 | 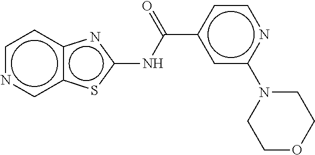 |
| 226 | 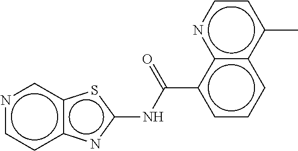 |
| 227 | 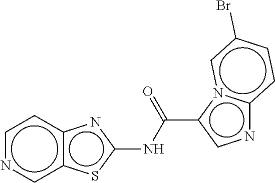 |
| 228 | 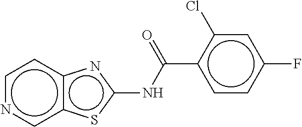 |
| 229 | 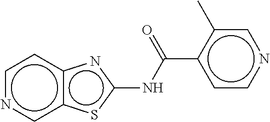 |
| 230 | 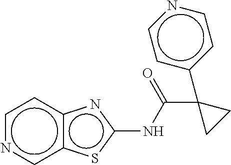 |
| 231 | 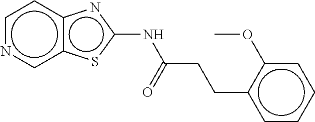 |
| 232 | 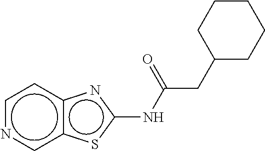 |
| 233 | 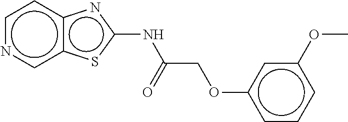 |
| 234 | 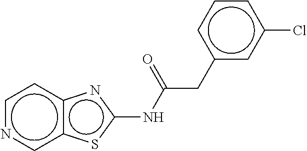 |
| 235 | 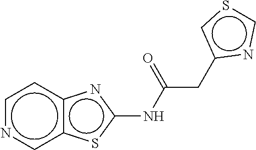 |
| 236 | 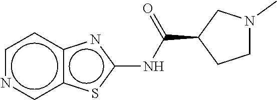 |
| 237 | 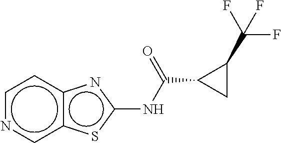 |
| 238 | 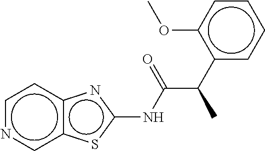 |
| 239 | 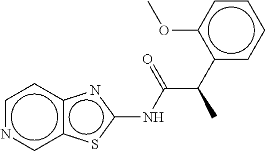 |
| 240 | 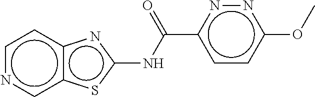 |
| 241 | 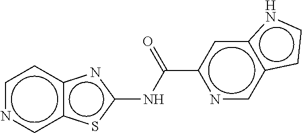 |
| 242 | 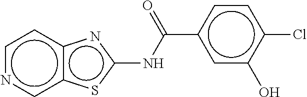 |
| 243 | 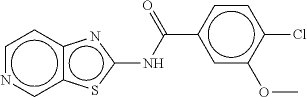 |
| 244 | 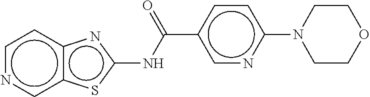 |
| 245 | 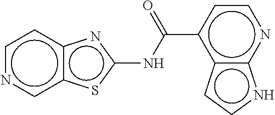 |
| 246 | 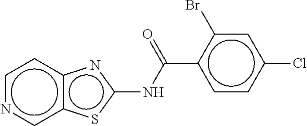 |
| 247 | 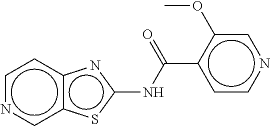 |
| 248 | 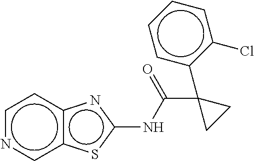 |
| 249 | 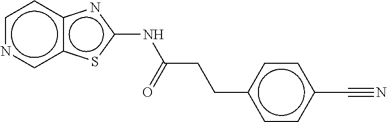 |
| 250 | 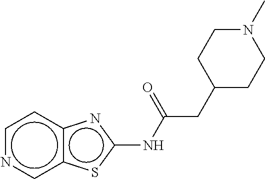 |
| 251 | 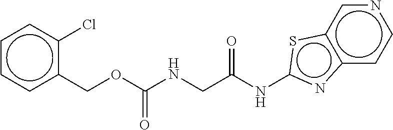 |
| 252 | 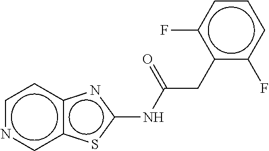 |
| 253 | 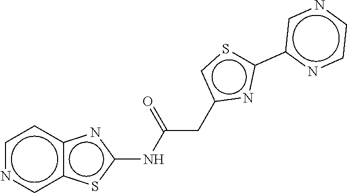 |
| 254 | 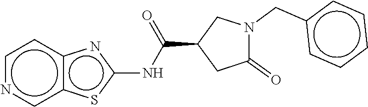 |
| 255 | 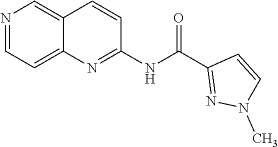 [SMILES: Cn1ccc(C(=O)Nc2ccc3cnccc3n2)n1] |
| 256 | 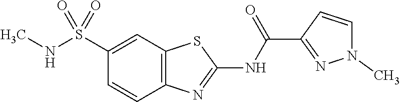 |

TABLE 2
Novel modulators of gp130 signaling

| | |
|---|---|
| A-1 | 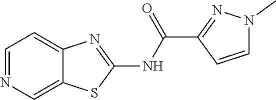 [SMILES: Cn1ccc(C(=O)Nc2nc3ccncc3s2)n1] |
| A-2 | 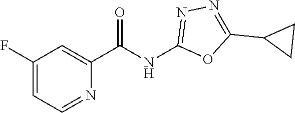 [SMILES: O=C(Nc1nnc(C2CC2)o1)c1cc(F)ccn1] |
| A-3 | 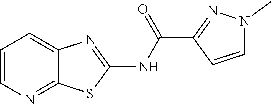 [SMILES: Cn1ccc(C(=O)Nc2nc3cccnc3s2)n1] |
| A-4 | 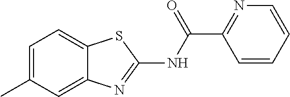 [SMILES: Cc1ccc2sc(NC(=O)c3ccccn3)nc2c1] |
| A-5 | 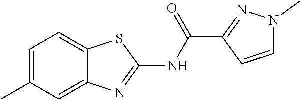 |
| A-6 | 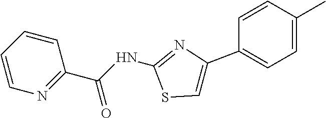 [SMILES: Cc1ccc(-c2csc(NC(=O)c3ccccn3)n2)cc1] |
| A-7 | 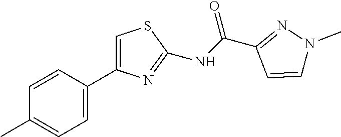 |
| A-8 | 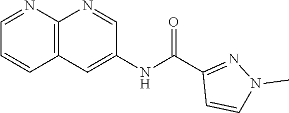 [SMILES: Cn1ccc(C(=O)Nc2cnc3ncccc3c2)n1] |
| A-9 | 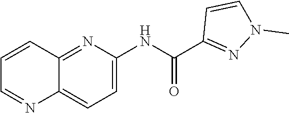 [SMILES: Cn1ccc(C(=O)Nc2ccc3ncccc3n2)n1] |
| A-10 | 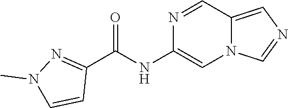 [SMILES: Cn1ccc(C(=O)Nc2cn3cncc3cn2)n1] |
| A-11 | 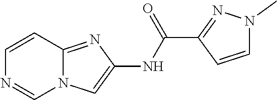 [SMILES: Cn1ccc(C(=O)Nc2cn3cnccc3n2)n1] |
| A-12 | 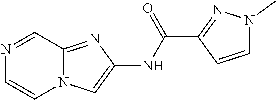 [SMILES: Cn1ccc(C(=O)Nc2cn3ccncc3n2)n1] |
| A-13 | 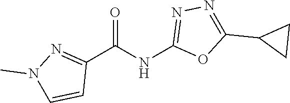 [SMILES: Cn1ccc(C(=O)Nc2nnc(C3CC3)o2)n1] |
| A-14 | 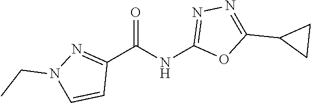 [SMILES: CCn1ccc(C(=O)Nc2nnc(C3CC3)o2)n1] |
| A-15 | 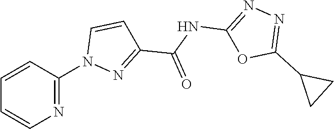 [SMILES: O=C(Nc1nnc(C2CC2)o1)c1ccn(-c2ccccn2)n1] |
| A-16 | 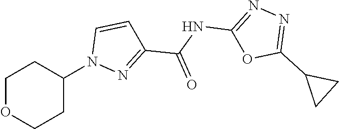 [SMILES: O=C(Nc1nnc(C2CC2)o1)c1ccn(C2CCOCC2)n1] |
| A-17 | 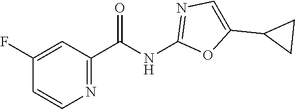 [SMILES: O=C(Nc1ncc(C2CC2)o1)c1cc(F)ccn1] |
| A-18 | 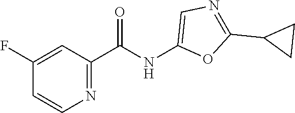 [SMILES: O=C(Nc1cnc(C2CC2)o1)c1cc(F)ccn1] |
| A-19 | 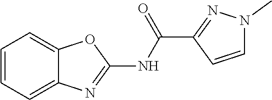 [SMILES: Cn1ccc(C(=O)Nc2nc3ccccc3o2)n1] |
| A-20 | 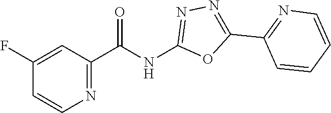 [SMILES: O=C(Nc1nnc(-c2ccccn2)o1)c1cc(F)ccn1] |
| A-21 | 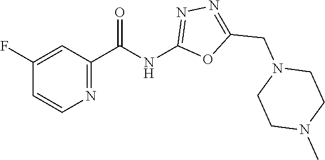 [SMILES: CN1CCN(Cc2nnc(NC(=O)c3cc(F)ccn3)o2)CC1] |
| A-22 | 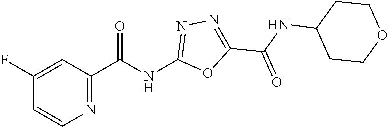 [SMILES: O=C(Nc1nnc(C(=O)NC2CCOCC2)o1)c1cc(F)ccn1] |
| A-23 | 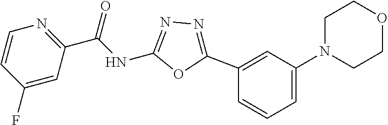 [SMILES: O=C(Nc1nnc(-c2cccc(N3CCOCC3)c2)o1)c1cc(F)ccn1] |
| A-24 | 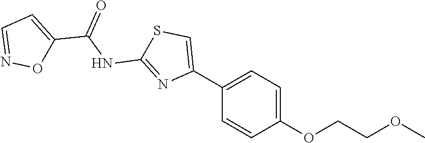 [SMILES: COCCOc1ccc(-c2csc(NC(=O)c3ccno3)n2)cc1] |
| A-25 | 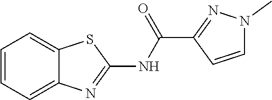 [SMILES: Cn1ccc(C(=O)Nc2nc3ccccc3s2)n1] |
| A-26 | 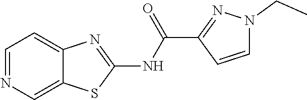 [SMILES: CCn1ccc(C(=O)Nc2nc3ccncc3s2)n1] |
| A-27 | 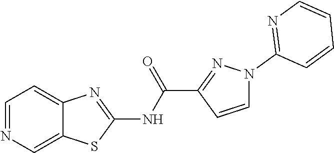 [SMILES: O=C(Nc1nc2ccncc2s1)c1ccn(-c2ccccn2)n1] |
| A-28 | 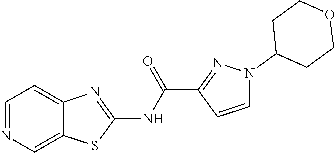 [SMILES: O=C(Nc1nc2ccncc2s1)c1ccn(C2CCOCC2)n1] |
| A-29 | 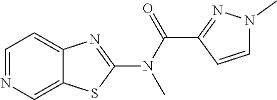 [SMILES: CN(C(=O)c1ccn(C)n1)c1nc2ccncc2s1] |
| A-30 | 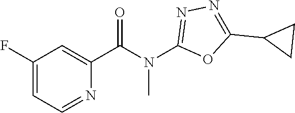 [SMILES: CN(C(=O)c1cc(F)ccn1)c1nnc(C2CC2)o1] |
| A-31 | 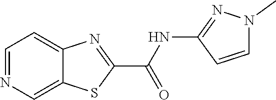 [SMILES: Cn1ccc(NC(=O)c2nc3ccncc3s2)n1] |
| A-32 | 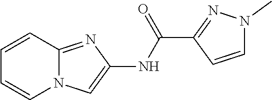 [SMILES: Cn1ccc(C(=O)Nc2cn3ccccc3n2)n1] |
| A-33 | 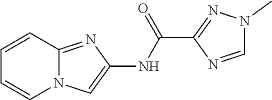 [SMILES: Cn1cnc(C(=O)Nc2cn3ccccc3n2)n1] |
| A-34 | 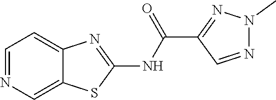 [SMILES: Cn1ncc(C(=O)Nc2nc3ccncc3s2)n1] |
| A-35 | 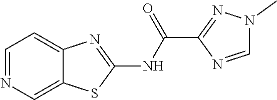 [SMILES: Cn1cnc(C(=O)Nc2nc3ccncc3s2)n1] |
| A-36 | 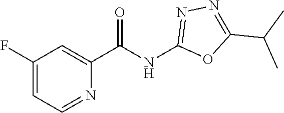 [SMILES: CC(C)c1nnc(NC(=O)c2cc(F)ccn2)o1] |
| A-37 | 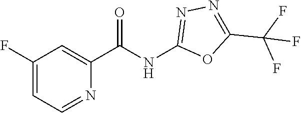 [SMILES: O=C(Nc1nnc(C(F)(F)F)o1)c1cc(F)ccn1] |
| A-38 | 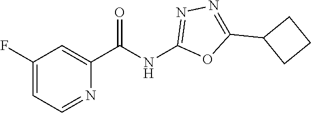 [SMILES: O=C(Nc1nnc(C2CCC2)o1)c1cc(F)ccn1] |
| A-39 | 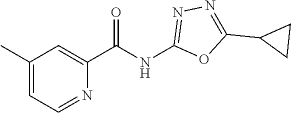 [SMILES: Cc1ccnc(C(=O)Nc2nnc(C3CC3)o2)c1] |
| A-40 | 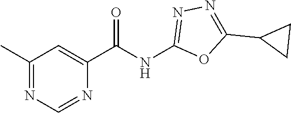 [SMILES: Cc1cc(C(=O)Nc2nnc(C3CC3)o2)ncn1] |
| A-41 | 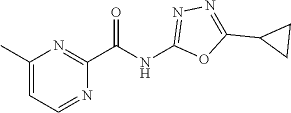 [SMILES: Cc1ccnc(C(=O)Nc2nnc(C3CC3)o2)n1] |
| A-42 | 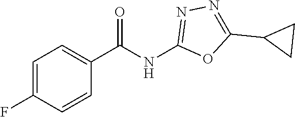 [SMILES: O=C(Nc1nnc(C2CC2)o1)c1ccc(F)cc1] |
| A-43 | 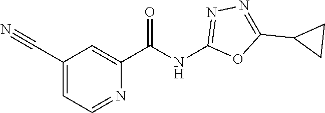 [SMILES: N#Cc1ccnc(C(=O)Nc2nnc(C3CC3)o2)c1] |
| A-44 | 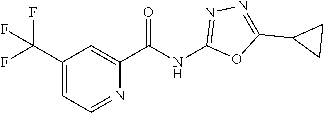 [SMILES: O=C(Nc1nnc(C2CC2)o1)c1cc(C(F)(F)F)ccn1] |
| A-45 | 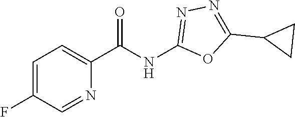 [SMILES: O=C(Nc1nnc(C2CC2)o1)c1ccc(F)cn1] |
| A-46 | 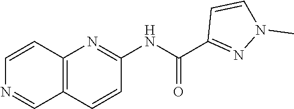 [SMILES: Cn1ccc(C(=O)Nc2ccc3cnccc3n2)n1] |
| A-47 | 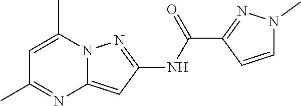 |
| A-48 | 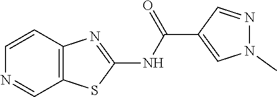 [SMILES: Cn1cc(C(=O)Nc2nc3ccncc3s2)cn1] |
| A-49 | 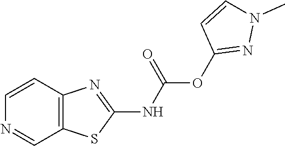 [SMILES: Cn1ccc(OC(=O)Nc2nc3ccncc3s2)n1] |
| A-50 | 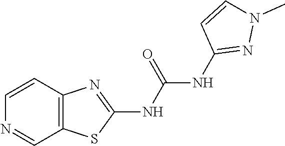 [SMILES: Cn1ccc(NC(=O)Nc2nc3ccncc3s2)n1] |
| A-51 | 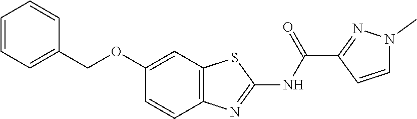 [SMILES: Cn1ccc(C(=O)Nc2nc3ccc(OCc4ccccc4)cc3s2)n1] |
| A-52 | 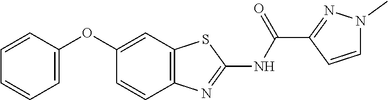 [SMILES: Cn1ccc(C(=O)Nc2nc3ccc(Oc4ccccc4)cc3s2)n1] |
| A-53 | 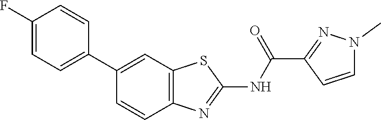 [SMILES: Cn1ccc(C(=O)Nc2nc3ccc(-c4ccc(F)cc4)cc3s2)n1] |
| A-54 | 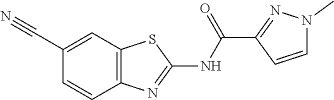 [SMILES: Cn1ccc(C(=O)Nc2nc3ccc(C#N)cc3s2)n1] |
| A-55 | 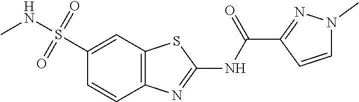 |
| A-56 | 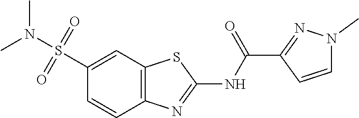 |
| A-57 | 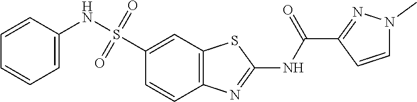 [SMILES: Cn1ccc(C(=O)Nc2nc3ccc(S(=O)(=O)Nc4ccccc4)cc3s2)n1] |
| A-58 | 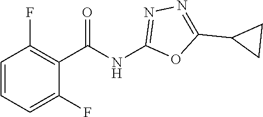 [SMILES: O=C(Nc1nnc(C2CC2)o1)c1c(F)cccc1F] |
| A-59 | 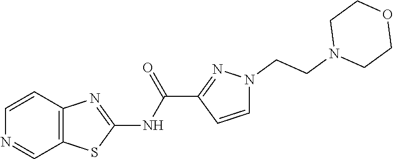 [SMILES: O=C(Nc1nc2ccncc2s1)c1ccn(CCN2CCOCC2)n1] |
| A-60 | 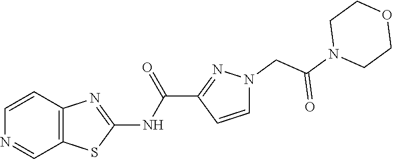 [SMILES: O=C(Nc1nc2ccncc2s1)c1ccn(CC(=O)N2CCOCC2)n1] |
| A-61 | 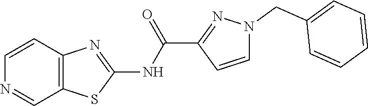 [SMILES: O=C(Nc1nc2ccncc2s1)c1ccn(Cc2ccccc2)n1] |
| A-62 | 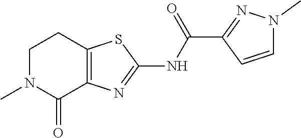 |
| A-63 | 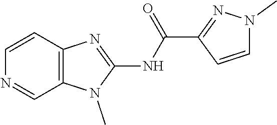 [SMILES: Cn1ccc(C(=O)Nc2nc3ccncc3n2C)n1] |
| A-64 | 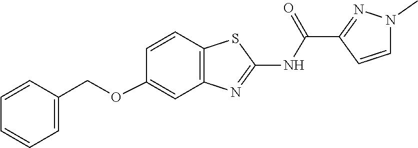 [SMILES: Cn1ccc(C(=O)Nc2nc3cc(OCc4ccccc4)ccc3s2)n1] |
| A-65 | 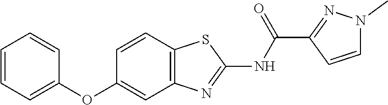 [SMILES: Cn1ccc(C(=O)Nc2nc3cc(Oc4ccccc4)ccc3s2)n1] |
| A-66 | 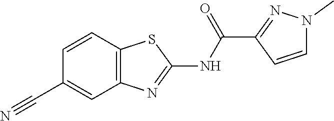 [SMILES: Cn1ccc(C(=O)Nc2nc3cc(C#N)ccc3s2)n1] |
| A-67 | 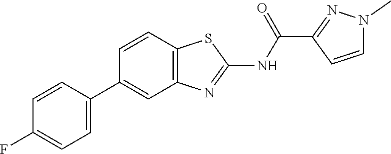 [SMILES: Cn1ccc(C(=O)Nc2nc3cc(-c4ccc(F)cc4)ccc3s2)n1] |
| A-68 | 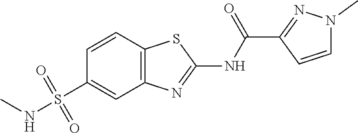 |
| A-69 | 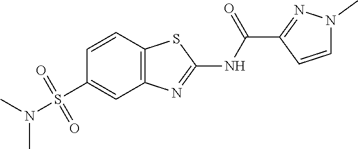 |
| A-70 | 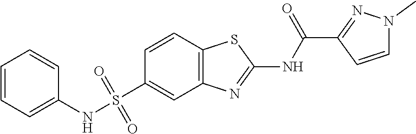 [SMILES: Cn1ccc(C(=O)Nc2nc3cc(S(=O)(=O)Nc4ccccc4)ccc3s2)n1] |
| A-71 | 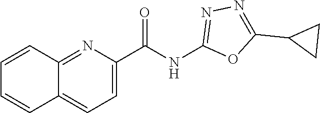 [SMILES: O=C(Nc1nnc(C2CC2)o1)c1ccc2ccccc2n1] |
| A-72 | 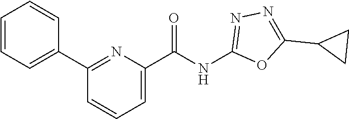 [SMILES: O=C(Nc1nnc(C2CC2)o1)c1cccc(-c2ccccc2)n1] |
| A-73 | 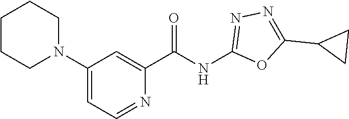 [SMILES: O=C(Nc1nnc(C2CC2)o1)c1cc(N2CCCCC2)ccn1] |
| A-74 | 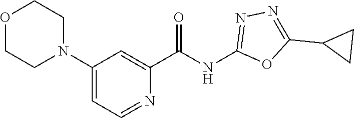 [SMILES: O=C(Nc1nnc(C2CC2)o1)c1cc(N2CCOCC2)ccn1] |
| A-75 | 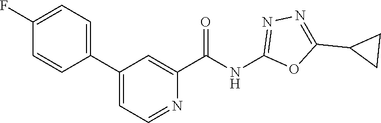 [SMILES: O=C(Nc1nnc(C2CC2)o1)c1cc(-c2ccc(F)cc2)ccn1] |
| A-76 | 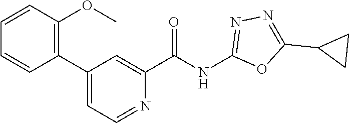 [SMILES: COc1ccccc1-c1ccnc(C(=O)Nc2nnc(C3CC3)o2)c1] |
| A-77 | 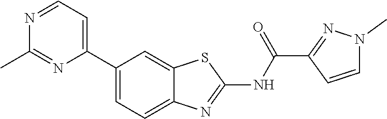 |
| A-78 | 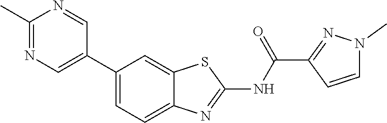 |
| A-79 | 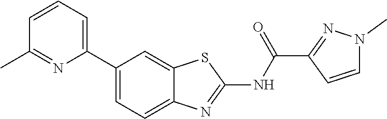 |
| A-80 | 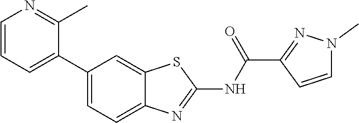 [SMILES: Cc1ncccc1-c1ccc2nc(NC(=O)c3ccn(C)n3)sc2c1] |
| A-81 | 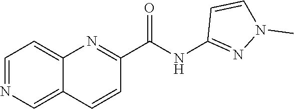 [SMILES: Cn1ccc(NC(=O)c2ccc3cnccc3n2)n1] |
| A-82 | 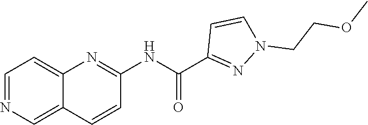 [SMILES: COCCn1ccc(C(=O)Nc2ccc3cnccc3n2)n1] |
| A-83 | 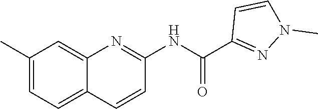 |
| A-84 | 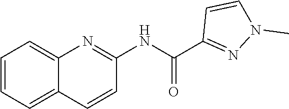 [SMILES: Cn1ccc(C(=O)Nc2ccc3ccccc3n2)n1] |
| A-85 | 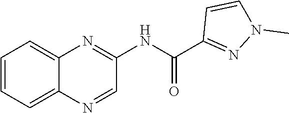 [SMILES: Cn1ccc(C(=O)Nc2cnc3ccccc3n2)n1] |
| A-86 | 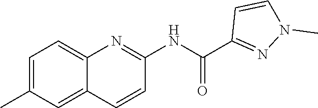 |
| A-87 | 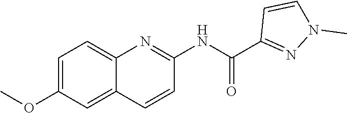 |
| A-88 | 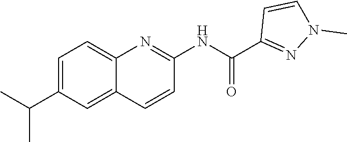 [SMILES: CC(C)c1ccc2nc(NC(=O)c3ccn(C)n3)ccc2c1] |
| A-89 | 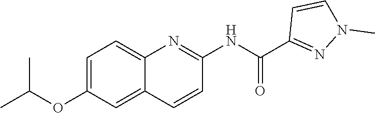 [SMILES: CC(C)Oc1ccc2nc(NC(=O)c3ccn(C)n3)ccc2c1] |
| A-90 | 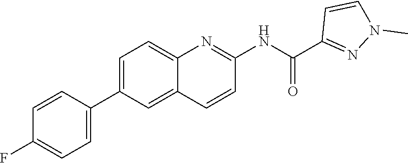 [SMILES: Cn1ccc(C(=O)Nc2ccc3cc(-c4ccc(F)cc4)ccc3n2)n1] |
| A-91 | 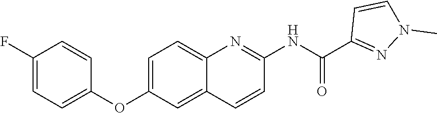 [SMILES: Cn1ccc(C(=O)Nc2ccc3cc(Oc4ccc(F)cc4)ccc3n2)n1] |
| A-92 | 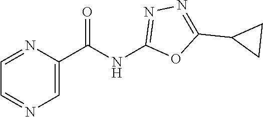 [SMILES: O=C(Nc1nnc(C2CC2)o1)c1cnccn1] |
| A-93 | 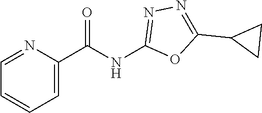 [SMILES: O=C(Nc1nnc(C2CC2)o1)c1ccccn1] |
| A-94 | 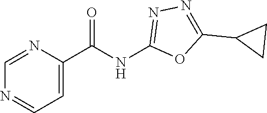 [SMILES: O=C(Nc1nnc(C2CC2)o1)c1ccncn1] |
| A-95 | 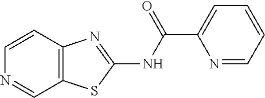 [SMILES: O=C(Nc1nc2ccncc2s1)c1ccccn1] |
| A-96 | 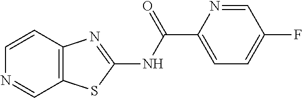 [SMILES: O=C(Nc1nc2ccncc2s1)c1ccc(F)cn1] |
| A-97 | 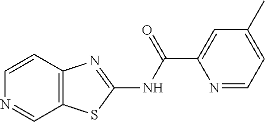 [SMILES: Cc1ccnc(C(=O)Nc2nc3ccncc3s2)c1] |
| A-98 | 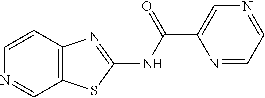 [SMILES: O=C(Nc1nc2ccncc2s1)c1cnccn1] |
| A-99 | 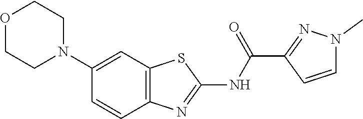 [SMILES: Cn1ccc(C(=O)Nc2nc3ccc(N4CCOCC4)cc3s2)n1] |
| A-100 | 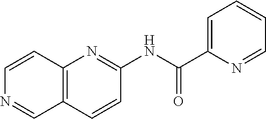 [SMILES: O=C(Nc1ccc2cnccc2n1)c1ccccn1] |
| A-101 | 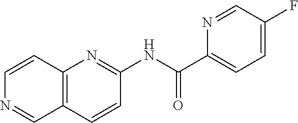 [SMILES: O=C(Nc1ccc2cnccc2n1)c1ccc(F)cn1] |
| A-102 | 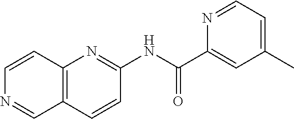 [SMILES: Cc1ccnc(C(=O)Nc2ccc3cnccc3n2)c1] |
| A-103 | 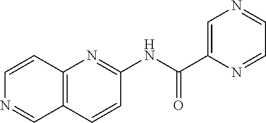 [SMILES: O=C(Nc1ccc2cnccc2n1)c1cnccn1] |
| A-104 | 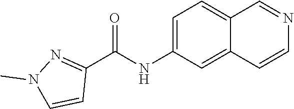 [SMILES: Cn1ccc(C(=O)Nc2ccc3cnccc3c2)n1] |
| A-105 | 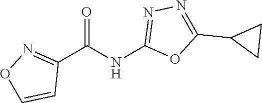 [SMILES: O=C(Nc1nnc(C2CC2)o1)c1ccon1] |
| A-106 | 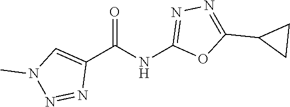 [SMILES: Cn1cc(C(=O)Nc2nnc(C3CC3)o2)nn1] |
| A-107 | 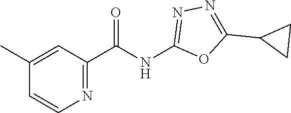 [SMILES: Cc1ccnc(C(=O)Nc2nnc(C3CC3)o2)c1] |
| A-108 | 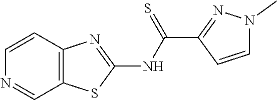 [SMILES: Cn1ccc(C(=S)Nc2nc3ccncc3s2)n1] |
| A-109 | 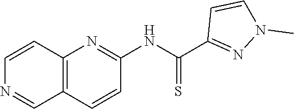 [SMILES: Cn1ccc(C(=S)Nc2ccc3cnccc3n2)n1] |
| A-110 | 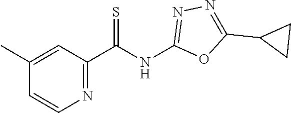 [SMILES: Cc1ccnc(C(=S)Nc2nnc(C3CC3)o2)c1] |
| A-111 | 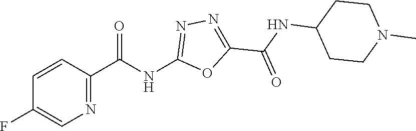 [SMILES: CN1CCC(NC(=O)c2nnc(NC(=O)c3ccc(F)cn3)o2)CC1] |
| A-112 | 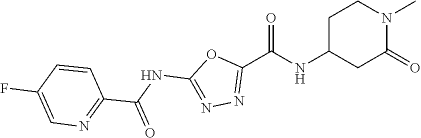 [SMILES: CN1CCC(NC(=O)c2nnc(NC(=O)c3ccc(F)cn3)o2)CC1=O] |
| A-113 | 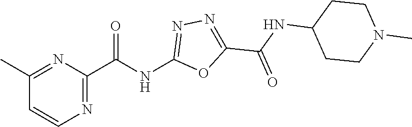 |
| A-114 | 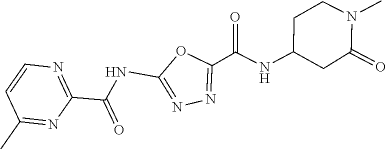 |
| A-115 | 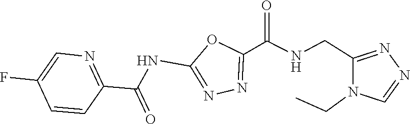 [SMILES: CCn1cnnc1CNC(=O)c1nnc(NC(=O)c2ccc(F)cn2)o1] |
| A-116 | 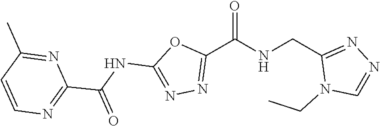 [SMILES: CCn1cnnc1CNC(=O)c1nnc(NC(=O)c2nccc(C)n2)o1] |
| A-117 | 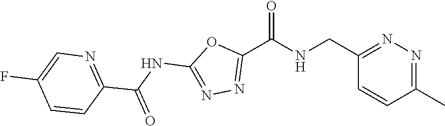 [SMILES: Cc1ccc(CNC(=O)c2nnc(NC(=O)c3ccc(F)cn3)o2)nn1] |
| A-118 | 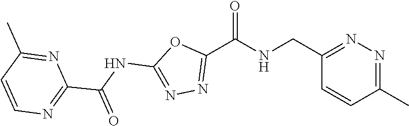 [SMILES: Cc1ccc(CNC(=O)c2nnc(NC(=O)c3nccc(C)n3)o2)nn1] |
| A-119 | 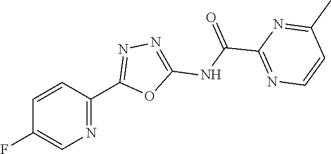 [SMILES: Cc1ccnc(C(=O)Nc2nnc(-c3ccc(F)cn3)o2)n1] |
| A-120 | 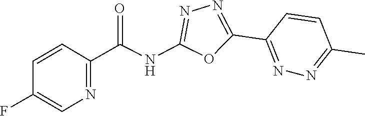 [SMILES: Cc1ccc(-c2nnc(NC(=O)c3ccc(F)cn3)o2)nn1] |
| A-121 | 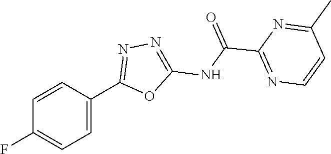 [SMILES: Cc1ccnc(C(=O)Nc2nnc(-c3ccc(F)cc3)o2)n1] |
| A-122 | 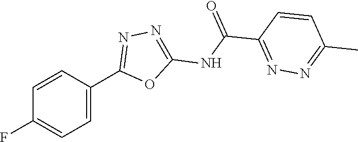 [SMILES: Cc1ccc(C(=O)Nc2nnc(-c3ccc(F)cc3)o2)nn1] |
| A-123 | 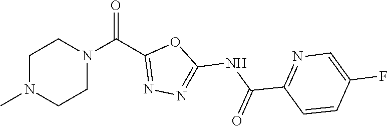 [SMILES: CN1CCN(C(=O)c2nnc(NC(=O)c3ccc(F)cn3)o2)CC1] |
| A-124 | 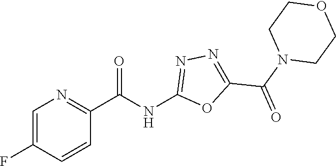 [SMILES: O=C(Nc1nnc(C(=O)N2CCOCC2)o1)c1ccc(F)cn1] |
| A-125 | 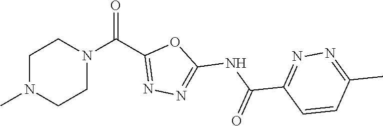 |
| A-126 | 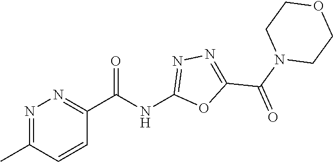 [SMILES: Cc1ccc(C(=O)Nc2nnc(C(=O)N3CCOCC3)o2)nn1] |
| A-127 | 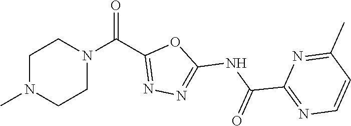 [SMILES: Cc1ccnc(C(=O)Nc2nnc(C(=O)N3CCN(C)CC3)o2)n1] |
| A-128 | 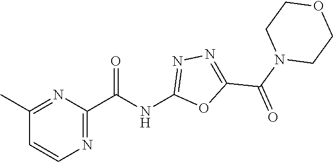 [SMILES: Cc1ccnc(C(=O)Nc2nnc(C(=O)N3CCOCC3)o2)n1] |
| A-129 | 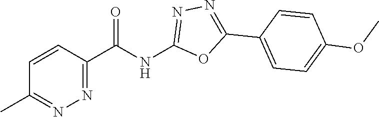 |
| A-130 | 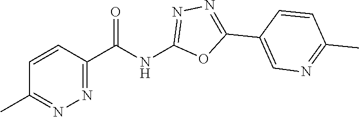 |
| A-131 | 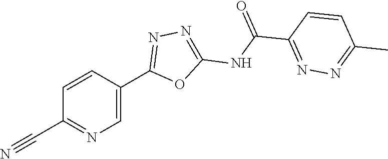 [SMILES: Cc1ccc(C(=O)Nc2nnc(-c3ccc(C#N)nc3)o2)nn1] |
| A-132 | 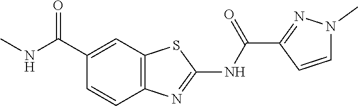 |
| A-133 | 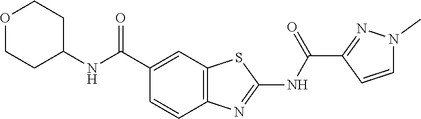 [SMILES: Cn1ccc(C(=O)Nc2nc3ccc(C(=O)NC4CCOCC4)cc3s2)n1] |
| A-134 | 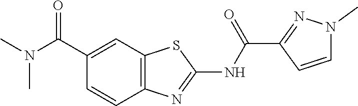 |

In one embodiment, the modulator modulates activity and/or expression of a molecule downstream of the gp130 signaling.

In another embodiment, the compound of the disclosure is a direct gp130 agonist.

In another embodiment, the modulator directly interacts with gp130 signal-transducing molecule.

In one aspect, the disclosed modulators have a unique pro-regenerative and anti-inflammatory profile, wherein the modulator inhibits (i) activation of p38, ERK1/2 and NF-kB by OSM, (ii) gp130 phosphorylation, (iii) MMP13 and ADAMTS4 expression induced by IL-6 family cytokines and (iv) promotes or does not inhibit YAP and/or STAT3 activation by LIF, wherein the modulator is capable of increasing expression of COL2 and ACAN in the presence of OSM.

In another aspect, the disclosed modulators have a unique anti-inflammatory profile, wherein the modulator inhibits (i) activation of p38, ERK1/2, NF-κb and gp130 by IL-6 family cytokines and (ii) MMP13 and ADAMTS4 expression induced by OSM.

In another aspect, the disclosed modulators have a unique pro-regenerative profile, wherein the modulator enhances YAP and/or STAT3 activation by LIF and the modulator is capable of increasing expression of COL2 and ACAN in the presence of OSM.

In some embodiments, the disclosed modulators regulate anti-inflammatory and/or pro-regenerative responses of the cell through modulating gp130 activity.

In one embodiment, the compound modulates the activity of SRC, NF-κB, YAP, p38, ERK1/2, STAT3 or MYC, or a combination thereof.

In one embodiment, the disclosed compounds modulate STAT3 and MYC signaling. In one embodiment, the compound does not stimulate STAT3 and MYC signaling. In another embodiment, the compound stimulates STAT3 and MYC signaling.

In one embodiment, the present invention also relates to stereoisomers, enantiomers, salts, solvates, polymorphs, multi-component complexes and liquid crystals of compounds of Formula (I) or (X-I) and subformula thereof.

In one embodiment, the present invention also relates to polymorphs and crystal habits of compounds of Formula (I) or (X-I) and subformula thereof, prodrugs and isomers thereof (including optical, geometric and tautomeric isomers) and isotopically-labeled compounds of Formula (I) or (X-I) and subformula thereof.

The compounds used in the present formulations may be in the form of pharmaceutically acceptable salts.

When a compound disclosed herein contains an acidic or basic moiety, it may also disclosed as a pharmaceutically acceptable salt (See, Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002).

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(IS)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oco-glutaric acid, glycolic acid, hippuric acid, hydrobromic acid, hydrochloric acid, hydroiodic acid, (+)-L-lactic acid, (+/−)-DL-lactic acid, lactobionic acid, lauric acid, maleic acid, (−)-L-malic acid, malonic acid, (+/−)-DL-mandelic acid, methanesulfonic acid, naphthalene-2-sulfonic acid, naphthalene-1,5-disulfonic acid, l-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, iV-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, iV-methyl-D-glucamine, 2-amino-2-(hydroxymethyl)-1, 3-propanediol, and tromethamine.

Compounds of Formula (I) or (X-I), and Tables 1 to 2 may be readily prepared. For instance, a nucleophilic reagent such as an amine including a primary or secondary amine may be reacted under suitable conditions with an acid or other reactive group to link portions of the compound. For instance, the nucleophilic reagent may be reacted with an acid (e.g., —C(═O)OH, —C(═O)halide such as —C(═O)Cl, —SO₃H) or other reactive group in one or more solvents including polar solvents such as methylene chloride, chloroform and the like at room temperature or elevated temperature for a time sufficient to effectively complete the reaction as may be determined by chromography or other analysis. The formed compound may be further functionalized as desired such as by one or more addition or coupling reactions. An exemplary preferred synthesis is shown in Example 7.

The invention also generally covers all pharmaceutically acceptable predrugs and prodrugs of the compounds of Formula (I) or (X-I) and subformula thereof.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

Prodrugs of the compounds are useful in the methods of this disclosure. Any compound that will be converted in vivo to provide a biologically, pharmaceutically or therapeutically active form of a compound of the disclosure is a prodrug. Various examples and forms of prodrugs are well known in the art. Examples of prodrugs are found, inter alia, in Design of Prodrugs, edited by H. Bundgaard, (Elsevier, 1985), Methods in Enzymology, Vol. 42, at pp. 309-396, edited by K. Widder, et al. (Academic Press, 1985); A Textbook of Drug Design and Development, edited by Krosgaard-Larsen and H. Bundgaard, Chapter 5, “Design and Application of Prodrugs,” by H. Bundgaard, at pp. 113-191, 1991); H. Bundgaard, Advanced Drug Delivery Reviews, Vol. 8, p. 1-38 (1992); H. Bundgaard, et al., Journal of Pharmaceutical Sciences, Vol. 77, p. 285 (1988); and Nogrady (1985) Medicinal Chemistry A Biochemical Approach, Oxford University Press, New York, pages 388-392).

Prodrugs of compounds disclosed herein can be prepared by methods known to one of skill in the art and routine modifications thereof, and/or procedures found in U.S. Pat. No. 8,293,786, and references cited therein and routine modifications made thereof.

### Pharmaceutical Compositions

Also provided herein are pharmaceutical formulations. The invention thus relates to a pharmaceutical composition comprising at least one modulator of gp130 signaling pathway, compound of any one of Formula (I), (X-I), (X-I-A), (X-I-B), (II), (III), (IV), (V), (V-a-j), (VI), (VIa-f), (VII), or (VIII) as described above, and at least one pharmaceutically acceptable carrier, diluent, excipient and/or adjuvant.

In one embodiment, a pharmaceutical composition comprises at least one modulator of gp130 signaling pathway selected from Tables 1 to 2, above, and at least one pharmaceutically acceptable carrier, diluent, excipient and/or adjuvant.

In embodiments of the pharmaceutical compositions, the compound, or pharmaceutically acceptable salt thereof, is included in a therapeutically effective amount.

A pharmaceutical composition of the disclosure is formulated to be compatible with its intended route of administration.

Examples of routes of administration include parenteral, e.g., intra-articular injection, intravenous, intradermal, subcutaneous, oral (e.g., inhalation), transdermal (topical), transmucosal, and rectal administration.

In one embodiment, the pharmaceutical composition of the invention comprising a compound of formula (I) or (X-I) as described above, is in a form suitable for administration to a subject. Such suitable administration form may be solid, semi-solid or liquid. Such suitable administration form will be clear to the skilled person; reference is made to the latest edition of Remington's Pharmaceutical Sciences.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy to administer by a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g. a compound of Formula (I), (X-I), (X-I-A), (X-I-B), (II), (III), (IV), (V), (V-a-j), (VI), (VIa-f), (VII), or (VIII) disclosed herein, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient plus any additional desired ingredient from a previously sterile-filtered solution thereof.

In a particular embodiment, one or more compounds of the disclosure are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to cells with monoclonal antibodies) can also be used as pharmaceutically acceptable carriers. These can be prepared according to methods known to those skilled in the art, for example, as described in U.S. Pat. No. 4,522,811.

In some embodiments, the pharmaceutical composition comprising compound of formula (I), (X-I), (X-I-A), (X-I-B), (II), (III), (IV), (V), (V-a-j), (VI), (VIa-f), (VII), or (VIII) is administered in solid form. Some preferred, but non-limiting examples of such forms include powders, tablets, pills, capsules, cachets, suppositories, and dispersible granules. In some embodiments, the pharmaceutical composition comprises compound of formula (I), (X-I), (X-I-A), (X-I-B), (II), (III), (IV), (V), (V-a-j), (VI), (VIa-f), (VII), or (VIII) and a solid carrier. A solid carrier may be one or more substance that may also act as diluents, flavoring agents, binders, preservatives, tablet disintegrating agents, or an encapsulating material. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 1% to 70% of the active compound of formula (I), (X-I), (X-I-A), (X-I-B), (II), (III), (IV), (V), (V-a-j), (VI), (VIa-f), (VII), or (VIII).

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

In one aspect, the pharmaceutical composition described herein is formulated to be compatible with topical administration. In one embodiment, the pharmaceutical composition is formulated as a topical formulation.

The topical formulation may be applied to a skin area following a suitable dosage and treatment regimen. The dosage and administration regimen for the described method is depend on the nature and condition being treated, the age and condition of the patient, and any prior or concurrent therapy.

In some instances, the topical formulation can be applied once every week, once every other day, once daily, twice daily, three times daily, or four time daily for a suitable period of time. The treatment may be terminated when the skin area is recovered. When necessary, the treatment may resume, for example, if a skin area needs additional treatment.

According to the invention, the topical formulations may be administered topically in the form of a cream, gel, or liquid. The topical administration provides the stabilized formulation directly to the skin, which is preferably provided with the use of a dermatologically acceptable carrier. While the carrier may consist of a relatively simple solvent or dispersant, such as an oil, it is generally preferred that the carrier comprises a material more conducive to topical application, and particularly one which will form a film or layer on the skin to which it is applied. This localizes the application and provides some resistance to perspiration and/or aids in percutaneous delivery and penetration of the active ingredients into lipid layers. Many such compositions are known in the art, and can take the form of creams, gels, ointments, hydrogels, pastes or plasters, and liquid dosage forms, such as solutions, emulsions, in particular oil-in-water emulsions, suspensions, for example lotions, etc., or even solid sticks. Liposomes or microspheres may also be used.

In some embodiments, the topical formulation may be administered using a device or method designed to more readily break the skin barrier and provide the agents in the topical formulation with a faster or more effective means through the stratum corneum. These include, for example, ultrasound therapy or ultrasound, oxygen nebulizers and nanosomal mist in conjunction with iontophoresis. In some embodiments, a spray or nebulizer may be used to create the nanosomel mist. In one embodiment, the micro-electronic cosmetic delivery mechanism described as PowerCosmetics® may be used for delivery of the topical formulation to the skin. This method is useful for delivering ionizable compounds to the skin and aids the penetration of small molecules through the stratum corneum.

In one embodiment, low intensity ultrasound delivery systems described as OZ Inside™ may be used for delivery of the topical formulation to the skin. PCT/US2011/041787, PCT/US2014/043951 are hereby incorporated by reference for using such delivery systems to administer the topical formulation of this disclosure.

The subject to be treated by the topical formulation can be a human or a non-human mammal.

In some embodiments, the pharmaceutical composition comprising compound of formula (I), (X-I), (X-I-A), (X-I-B), (II), (III), (IV), (V), (V-a-j), (VI), (VIa-f), (VII), or (VIII) may be administered orally in solid form. Some preferred, but non-limiting examples of such forms include powders, tablets, pills, capsules, cachets, suppositories, and dispersible granules.

In one aspect, the pharmaceutical composition comprising compound of formula (I), (X-I), (X-I-A), (X-I-B), (II), (III), (IV), (V), (V-a-j), (VI), (VIa-f), (VII), or (VIII) may be formulated for use in implant coating.

In some embodiments, implant coating is used for coating an implantable device.

In some embodiments, implant coating is useful for slow release of the pharmaceutical composition comprising compound of formula (I), (X-I), (X-I-A), (X-I-B), (II), (III), (IV), (V), (V-a-j), (VI), (VIa-f), (VII), or (VIII).

In some embodiments, the implantable device is a member selected from the group consisting of a bone substitute, a joint prosthesis, a dental implant, a maxillofacial implant, a vertebral surgery aid, and a transcutaneous device.

### Further Ingredients

The pharmaceutical composition of the invention may optionally comprise one or more other pharmaceutically acceptable carrier, diluent, excipient and/or adjuvant. Such suitable carrier, diluent, excipient and/or adjuvant for use in the preparation of the administration forms will be clear to the skilled person; reference is made to the latest edition of Remington's Pharmaceutical Sciences.

Especially, the pharmaceutical composition of the invention can optionally contain such inactive substances that are commonly used in pharmaceutical formulations, such as for example cosolvents, antioxidants, surfactants, wetting agents, emulsifying agents, buffering agents, pH modifying agents, preserving agents (or preservating agents), isotonifiers, stabilizing agents, granulating agents or binders, precipitation inhibitors, lubricants, disintegrants, glidants, diluents or fillers, adsorbents, dispersing agents, suspending agents, bulking agents, release agents, sweetening agents, flavoring agents, and the like.

According to one embodiment, the pharmaceutical composition of the invention comprises one or more pharmaceutically acceptable inactive ingredients selected from: caprylic acid, polyethylene glycol, propylene glycol, ethanol, glycerol, dimethylsulfoxide, dimethylacetamide, dimethylisosorbide, cellulose derivatives (including hydroxypropylmethylcellulose, methylcellulose, hydroxypropylcellulose, hydroxypropylmethylcellulose phthalate and hydroxypropylmethylcellulose acetate succinate), cremophor RH40 (polyoxyl 40 hydrogenated castor oil), cremophor EL (polyoxyl 35 hydrogenated castor oil), polysorbate 20 (polyoxyethylenesorbitan monolaurate), polysorbate 80 (polyoxyethylenesorbitan monooleate), poloxamer 188 (poly(ethylene glycol)-block-poly(propylene glycol)-block-poly(ethylene glycol)), poloxamer 407 (Poly(ethylene glycol)-block-poly(propylene glycol)-block-poly(ethylene glycol)), vitamin E TPGS (vitamin E polyethylene glycol succinate), solutol HS15 (polyoxyethylated 12-hydroxystearic acid), labrasol (caprylocaproyl polyoxyl-8 glycerides), labrafil M1944 (Oleoyl polyoxyl-6 glycerides), polyvinylpyrrolidone (also called povidone, preferably polyvinylpyrrolidone K17, K19, K29-K32, K90), polyvinylpyrrolidone polyvinylacetate copolymer, carboxymethylcellulose (Na/Ca), polyethylene glycol methyl ether-block-poly(D-L-lactide) copolymer, sodium lauryl sulfate, sodium docusate, propylene glycol monolaurate, propylene glycol dilaurate, propylene glycol monocaprylate, polyethylene glycol 660 12-monostearate, poly(butyl methacrylate-co-(2-dimethylaminoethyl) methacrylate-co-methyl methacrylate) 1:2:1, sodium lauryl sulphate.

In a preferred embodiment, the pharmaceutical composition of the invention comprises one or more pharmaceutically acceptable cosolvents. Preferably cosolvents are selected from caprylic acid, polyethylene glycol (PEG), propylene glycol, ethanol, dimethylsulfoxide, dimethylacetamide, dimethylisosorbide and mixtures thereof. In a specific embodiment, the pharmaceutical composition of the invention comprises caprylic acid and/or PEG. Advantageously, when the composition comprises PEG as cosolvent, PEG is of low molecular weight, preferably PEG is PEG 400. In an alternative embodiment, when the composition comprises PEG, it is of a moderate molecular weight, preferably PEG 2000.

In one embodiment, the pharmaceutical composition of the invention further comprises one or more antioxidant; preferably the antioxidant is selected from butylated hydroxytoluene (BHT), butylated hydroxyanisole (BHA), citric acid, sodium metabisulfite, ascorbic acid, methionine and vitamin E; more preferably the antioxidant is BHT.

In some embodiments, surfactants are added, such as for example polyethylene glycols, polyoxyethylene sorbitan fatty acid esters, sorbitan esters, sodium docusate, sodium lauryl sulfate, polysorbates (20, 80, etc.), poloxamers (188, 407 etc.), pluronic polyols, polyoxyethylene sorbitan monoethers (TWEEN®-20, TWEEN®-80, etc.), vitamin E TPGS (Vitamin E polyethylene glycol succinate), cremophor RH40 (polyoxyl 40 hydrogenated castor oil), cremophor EL (polyoxyl 35 hydrogenated castor oil), polyethylene glycol 660 12-monostearate, solutol HS15 (Polyoxyethylated 12-hydroxystearic acid), labrasol (caprylocaproyl polyoxyl-8 glycerides), labrafil M1944 (Oleoyl polyoxyl-6 glycerides).

In some embodiments, wetting agents are added, such as for example sodium lauryl sulphate, vitamin E TPGS, sodium docusate, polysorbate 80, poloxamer 407. A preferred wetting agent id sodium lauryl sulphate.

In some embodiments, emulsifying agents are added, such as for example carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate. Preferred emulsifying agents are for example poloxamer, propylene glycol monolaurate, propylene glycol dilaurate, and propylene glycol monocaprylate.

In some embodiments, buffering agents are used to help to maintain the pH in the range that approximates physiological conditions Suitable buffering agents include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

In some embodiments, pH modifiers are added, such as for example sodium hydroxide, sodium bicarbonate, magnesium oxide, potassium hydroxide, meglumine, sodium carbonate, citric acid, tartaric acid, ascorbic acid, fumaric acid, succinic acid and malic acid.

In some embodiments, preservatives agents are added to retard microbial growth. Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol.

In some embodiments, isotonifiers sometimes known as “stabilizers” are added and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active ingredient. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); monosaccharides, such as xylose, mannose, fructose, glucose; disaccharides such as lactose, maltose, sucrose and trisaccacharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. Preferred stabilizers are for example glycerol; polyethylene glycol; polyvinylpyrrolidone; cellulose derivatives such as hydroxypropylmethylcellulose, hydroxypropylmethylcellulose phthalate or hydroxypropylmethylcellulose acetate succinate; carboxymethylcellulose (Na/Ca); polyethylene glycol methyl ether-block-poly(D-L-lactide) copolymer; and poly(butyl methacrylate-co-(2-dimethylaminoethyl) methacrylate-co-methyl methacrylate) 1:2:1.

In some embodiments granulating agent/binder(s) are added, such as for example starch, gums (inclusive of natural, semisynthetic and synthetic), microcrystalline cellulose, ethyl cellulose, methylcellulose, hydroxypropylcellulose, liquid glucose polymers such as povidone, polyvinylpyrrolidone polyvinylacetate copolymer and the like. Preferred granulating agents are for example methylcellulose, hydroxypropylcellulose, povidone and polyvinylpyrrolidone polyvinylacetate copolymer.

In some embodiments precipitation inhibitors are added, such as for example water soluble derivatives of cellulose including hydroxypropylmethylcellulose and methylcellulose, and water soluble polymers such as polyvinylpyrrolidone or polyvinylpyrrolidone polyvinylacetate copolymer. A preferred precipitation inhibitor is hydroxypropylmethylcellulose.

In some embodiments lubricants are added, such as for example magnesium stearate, glyceryl esters, behenoyl polyoxyl-8 glycerides Nf (Compritol HD5 ATO), sodium stearyl fumarate and the like.

In some embodiments disintegrants are added, such as for example synthetics like sodium starch glycolate, cross povidone, cross carmellose sodium, kollidon CL, and natural origin such as locust bean gum and the like.

In some embodiments glidants are added, such as for example talc, magnesium stearate, colloidal silicon dioxide, starch and the like.

In some embodiments, diluents (or fillers) are added, such as for example dextrose, lactose, mannitol, microcrystalline cellulose, sorbitol, sucrose, dibasic calcium phosphate, calcium sulphate dehydrate, starch and the like.

In some embodiments, adsorbents are added, such as for example silicon dioxide, purified aluminium silicate and the like.

In some embodiments, the pharmaceutical composition of the invention is in the form of tablets and tableting excipients are added, such as for example granulating agents, binders, lubricants, disintegrants, glidants, diluents, adsorbents and the like.

In some embodiments, the pharmaceutical composition of the invention is in the form of capsules, in which the capsule shells are constructed from gelatin or from non-animal derived products such as cellulose and its derivatives such as hydroxypropylmethylcellulose. Other ingredients may be included in the capsule shells such as polyethyleneglycol to act as plasticizer; pigments such as titanium dioxide or iron oxide to provide opacity and colour differentiation; lubricants such as carnauba wax; gelling agents such as carrageenan and wetting agents such as sodium lauryl sulphate. In one embodiment, the pharmaceutical composition of the invention is formulated as capsules, wherein the capsule shells are constructed from gelatin and wherein additional components are optionally included in the capsule shells, such as for example polyethylene glycol and sodium lauryl sulphate.

### Dosages and Unit Dose

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC50 (e.g., the concentration of the test compound which achieves a half-maximal inhibition of symptoms) as determined in cell culture. Such information can be used to more accurately determine useful doses in humans. Levels in plasma can be measured, for example, by high performance liquid chromatography.

The pharmaceutical preparation is preferably in unit dosage form. In such form the

preparation is subdivided into unit doses containing appropriate quantities of the active component.

The unit dosage form can be a packaged preparation, the package containing discrete

quantities of preparation, such as packeted tablets, capsules, and powders in vials or ampoules.

Also, the unit dosage form can be a capsule, tablet, cachet, or lozenge itself, or it can be the appropriate number of any of these in packaged form.

The quantity of compound of Formula (I), (X-I), (X-I-A), (X-I-B), (II), (III), (IV), (V), (V-a-j), (VI), (VIa-f), (VII), or (VIII) in a unit dose preparation may be varied or adjusted according to the particular application and the potency of the active component. In one embodiment, the dosage comprises the compound in an amount from about 0.1 mg to about 10000 mg. In another embodiment the dosage comprises the compound in an amount from about 1 mg to about 1000 mg. In another embodiment the dosage comprises the compound in an amount from about 1 mg to about 400 mg. In another embodiment the dosage comprises the compound in an amount from about 1 mg to about 100 mg. In another embodiment the dosage comprises the compound in an amount from about 1 mg to about 10 mg.

The composition can, if desired, also contain other compatible therapeutic agents. Some compounds of formula (I), (X-I), (X-I-A), (X-I-B), (II), (III), (IV), (V), (V-a-j), (VI), (VIa-f), (VII), or (VIII) may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

In one embodiment, the pharmaceutical composition of the invention is administered as a daily dose such that it corresponds administering about 1 mg to about 400 mg of compound of Formula (I), (X-I), (X-I-A), (X-I-B), (II), (III), (IV), (V), (V-a-j), (VI), (VIa-f), (VII), or (VIII) described above to the subject per day.

In some embodiments, daily dosage is administered in separate administrations of 2, 3, 4, or 6 equal unit doses throughout the day.

In another embodiment, daily dosage is administered as a single unit dose.

In some embodiments, each unit dose is administered in the form of one, two, three, or four tablet, suspension, granule or capsule.

The pharmaceutical composition of the invention may also be formulated so as to provide rapid, sustained or delayed release of the modulator described above contained therein.

### Additional Therapeutic Agents and Methods

Compositions and formulations of one or more modulators of gp130 signaling pathway disclosed herein can be used in combination with other active agents to treat a disorder or disease in a subject.

It should be understood that the administration of an additional therapeutic agent with a compound of the disclosure encompasses co-administration of these therapeutic agents in a substantially simultaneous manner, such as in a single capsule having a fixed ratio of active ingredients or in multiple, separate capsules for each active ingredient. In addition, administration of an additional therapeutic agent in combination with a compound disclosed herein also encompasses use of each type of therapeutic agent in a sequential manner. In either case, the treatment regimen will provide beneficial effects of the drug combination in treating the disorders described herein.

In a further embodiment, the compounds disclosed herein can be combined with one or more class of therapeutic agents, including, but not limited to, alkylating agents, cancer immunotherapy monoclonal antibodies, anti-metabolites, mitotic inhibitors, antitumor antibiotics, topoisomerase inhibitors, photosensitizers, tyrosine kinase inhibitors, anti-cancer agents, chemotherapeutic agents, anti-migraine treatments, anti-tussives, mucolytics, decongestants, anti-allergic non-steroidals, expectorants, antihistamine treatments, anti-retroviral agents, CYP3A inhibitors, CYP3A inducers, protease inhibitors, adrenergic agonists, anticholinergics, mast cell stabilizers, xanthines, leukotriene antagonists, glucocorticoid treatments, antibacterial agents, antifungal agents, sepsis treatments, steroidals, local or general anesthetics, NSAIDS, NRIs, DARIs, SNRIs, sedatives, NDRIs, SNDRIs, monoamine oxidase inhibitors, hypothalamic phoshpholipids, antiemetics, ECE inhibitors, opioids, thromboxane receptor antagonists, potassium channel openers, thrombin inhibitors, growth factor inhibitors, anti-platelet agents, P2Y (AC) antagonists, anticoagulants, low molecular weight heparins, Factor Via inhibitors, Factor Xa inhibitors, renin inhibitors, NEP inhibitors, vasopepsidase inhibitors, squalene synthetase inhibitors, anti-atherosclerotic agents, MTP inhibitors, calcium channel blockers, potassium channel activators, alpha-muscarinic agents, beta-muscarinic agents, anti-arrhythmic agents, diuretics, thrombolytic agents, anti-diabetic agents, mineralocorticoid receptor antagonists, growth hormone secretagogues, aP2 inhibitors, phophodiesterase inhibitors, anti-inflammatories, antiproliferatives, antibiotics, farnesyl-protein transferase inhibitors, hormonal agents, plant-derived products, epipodophyllotoxins, taxanes, prenyl-protein transferase inhibitors, anti-TNF antibodies and soluble TNF receptors, Cyclooxygenase-2 inhibitors, and miscellaneous agents.

It should be understood that the administration of an additional therapeutic agent with a compound of the disclosure encompasses co-administration of these therapeutic agents in a substantially simultaneous manner, such as in a single capsule having a fixed ratio of active ingredients or in multiple, separate capsules for each active ingredient. In addition, administration of an additional therapeutic agent in combination with a compound disclosed herein also encompasses use of each type of therapeutic agent in a sequential manner. In either case, the treatment regimen will provide beneficial effects of the drug combination in treating the disorders described herein.

### Kits

For use in the therapeutic applications described herein, kits and articles of manufacture are also described herein. Such kits can comprise a carrier, package, or container that is compartmentalized to receive one or more containers such as vials, tubes, and the like, each of the container (s) comprising one of the separate elements to be used in a method described herein. Suitable containers include, for example, bottles, vials, syringes, and test tubes. The containers can be formed from a variety of materials such as glass or plastic.

For example, the container (s) can comprise one or more compounds described herein, optionally in a composition or in combination with another agent as disclosed herein. The container (s) optionally have a sterile access port (for example the container can be an intravenous solution bag or a vial having a stopper pierceable by a hypodermic injection needle). Such kits optionally comprise a compound with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

### Topical Formulation

The present invention also generally covers all provides novel topical formulation of a small molecule modulator of gp130 signaling or a derivative thereof for improvement of skin appearance in normal and aged human skin.

The one embodiment the topical formulation has an appearance of opaque and viscous lotion.

In another embodiment the topical formulation has a color of off-white to straw.

In another embodiment the topical formulation has an odor of citrus.

In another embodiment the topical formulation has a specific gravity at 25° C.

In another embodiment the topical formulation has a pH at 25° C. at a range of 4.5-5.5

In another embodiment the topical formulation has percentage of total solids at a range of 17.5% to 21.5%

In another embodiment the topical formulation has a viscosity at 25° C. at a range of 18,000 centipoises (cps)-40,000 cps.

In one embodiment, excipients commonly found in skin care compositions such as, for example, emollients, skin conditioning agents, emulsifying agents, humectants, preservatives, antioxidants, perfumes, chelating agents, buffering agents, etc. may be utilized provided that they are physically and chemically compatible with other components of the formulation.

The one embodiment, the topical formulation contains:
a. a first part that contains a mixture of a first component, a second component, a third component a fourth component, a fifth component, and a sixth component;
b. a second part that contains a mixture of two components, a seventh component and an eighth component; and
c. a third part that contains a mixture of a ninth component, a 10th component, an 11th component, a 12th component, a 13th component, a 14th component, a 15th component, a 16th component, a 17th component, and an 18th component, wherein the formulation contains a combination of the first part, the second part, and the third part.

In one embodiment, the topical formulation comprises a crosslinked hyaluronic acid. In one embodiment, the crosslinked hyaluronic acid is an aqueous combination of pentylene glycol, ethylhexylglycerin, sodium hyaluronate, and a crosspolymer, e.g., Hylasome® EG10 (CAS #105524-32-1, available from Vantage Specialty Ingredients). In one embodiment, the topical formulation comprises the crosslinked hyaluronic acid at about 1% W/W. In one embodiment, the topical formulation comprises the crosslinked hyaluronic acid at about 10% W/W.

In one embodiment, the topical formulation comprises a self-emulsifying elastomer dispersion. In one embodiment, the self-emulsifying elastomer dispersion is a combination of dimethicone, polysilicone-11, isohexadecane, ammonium polyacryloyldimethyl taurate, tocopheryl acetate, polysorbate 80, and polysorbate 20, e.g., Gransil ORB-5CS (available from Grant Industries). In one embodiment, the topical formulation comprises the self-emulsifying elastomer dispersion at about 25% W/W. In one embodiment, the topical formulation comprises the self-emulsifying elastomer dispersion at about 25% W/W.

In one embodiment, the topical formulation comprises a branched aliphatic hydrocarbon emollient. In one embodiment, the branched aliphatic hydrocarbon emollient is an isododecane, e.g., Armesiil2C (CAS #93685-81-5, #31807-55-3, #13475-82-6, available from Argan Co.). In one embodiment, the topical formulation comprises the branched aliphatic hydrocarbon emollient at about 8% W/W. In one embodiment, the topical formulation comprises the branched aliphatic hydrocarbon emollient at about 8% W/W.

In one embodiment, the topical formulation comprises a stable and oil soluble form of vitamin C. In one embodiment, the stable and oil soluble form of vitamin C is tetrahexyldecyl ascorbate, e.g., BV—OSC (CAS #183476-82-6, available from Barnet Products Corporation). In one embodiment, the topical formulation comprises the stable and oil soluble form of Vitamin C at about 0.5% W/W. In one embodiment, the topical formulation comprises the stable and oil soluble form of vitamin C about 0.5% W/W.

In one embodiment, the topical formulation comprises an active cooling ingredient. In one embodiment, the active cooling ingredient is menthyl ethylamido oxalate, e.g., Frescolat® X-Cool (available from Symrise). In one embodiment, the topical formulation comprises the stable and oil soluble form of vitamin C at about 0.1% W/W. In one embodiment, the topical formulation comprises the active cooling ingredient at about 0.10% W/W.

In one embodiment, the topical formulation comprises orange essential oil blend. In one embodiment, the orange essential oil blend is a combination of limonene, citrus Aurantium dulcis (orange), peel oil and Citrus tangerina (tangerine) peel oil, e.g., Frag. Orange Essential Oil Blend (#CE-188609, available from Harris). In one embodiment, the topical formulation comprises the stable and oil soluble form of vitamin C at about 0.2% W/W. In one embodiment, the topical formulation comprises the orange essential oil blend at about 0.2% W/W.

In one embodiment, the topical formulation comprises a viscous oil. In one embodiment, the viscous oil is tocopherol e.g., DL-Alpha Tocopherol (available from DSM). In one embodiment, the topical formulation comprises the viscous oil at about 0.1% W/W. In one embodiment, the topical formulation comprises the viscous oil at about 0.1% W/W.

In one embodiment, the topical formulation comprises an emollient. In one embodiment, the emollient is butylene glycol, e.g., 1,3-butylene glycol (available from Univar). In one embodiment, the topical formulation comprises the emollient at about 4% W/W. In one embodiment, the topical formulation comprises the emollient at about 4% W/W.

In one embodiment, the topical formulation comprises a polymer thickener and stabilizing agent. In one embodiment, the polymer thickener and stabilizing agent is a combination of isohexadecane, ammonium polyacryloyldimethyl taurate, polysorbate 80 e.g., Granthix APP (available from Grant Industries). In one embodiment, the topical formulation comprises the polymer thickener and stabilizing agent at about 2.5% W/W. In one embodiment, the topical formulation comprises the polymer thickener and stabilizing agent at about 2.5% W/W.

In one embodiment, the topical formulation comprises a sustainable pentylene glycol. In one embodiment, the sustainable pentylene glycol is Hydrolite® 5 green (available from Symrise). In one embodiment, the topical formulation comprises the sustainable pentylene glycol at about 1% W/W. In one embodiment, the topical formulation comprises the sustainable pentylene glycol at about 1% W/W.

In one embodiment, the topical formulation comprises a lipopeptide. In one embodiment, the lipopeptide is a combination of water (aqua), pentylene glycol, caprylyl glycol, N-prolyl palmitoyl tripeptide-56 acetate e.g., Matrixyl® Morphemics™ (available from Sederma). In one embodiment, the topical formulation comprises the lipopeptide at about 2% W/W. In one embodiment, the topical formulation comprises the lipopeptide at about 2% W/W.

In one embodiment, the topical formulation comprises a stress oxidative inhibitor. In one embodiment, the stress oxidative inhibitor is a combination of glycerin and acer rubrum extract e.g., Borealine Expert (available from Lucas Meyer). In one embodiment, the topical formulation comprises the lipopeptide at about 0.1% W/W. In one embodiment, the topical formulation comprises the stress oxidative inhibitor at about 0.1% W/W.

In one embodiment, the topical formulation comprises an extract from tropical fruits standardized in carbohydrates and total alpha hydroxyacids (AHAs). In one embodiment, the extract from tropical fruits is a combination of water (aqua), Spondias mombin pulp extract, Mangifera indica (mango) pulp extract, Musa sapientum (banana) pulp extract, benzyl alcohol, potassium sorbate e.g., Exfo-Bio (available from ChemyUnion). In one embodiment, the topical formulation comprises the extract from tropical fruits at about 2% W/W. In one embodiment, the topical formulation comprises the extract from tropical fruits standardized in carbohydrates and total alpha hydroxyacids (AHAs) at about 2% W/W.

In one embodiment, the topical formulation comprises a blend of multifunctional ingredients with self-preserving properties. In one embodiment, the blend of multifunctional ingredients comprises a combination of glyceryl caprylate, glycerin, capryl-hydroxamic acid e.g., Spectrastat™ G2N (available from Inolex). In one embodiment, the topical formulation comprises the blend of multifunctional ingredients at about 1.2% W/W. In one embodiment, the topical formulation comprises the blend of multifunctional ingredients with self-preserving properties at about 1.2% W/W.

In one embodiment, the topical formulation comprises at least one chelating agent. The term “chelating agent” as used herein refers to any known pharmaceutically acceptable chelating agents. Suitable chelating agents can include but are not limited to any one or more of ethylenediaminetetraacetic acid (EDTA) and derivatives thereof, ethylene glycol-bis-(2-aminoethyl)-N,N,N′,N′-tetraacetic acid (EGTA) and derivatives thereof, cyclohexanediamine tetraacetic acid (CDTA) and derivatives thereof, hydroxyethylethylenediamine triacetic acid (HEDTA) and derivatives thereof, diethylenetriamine pentaacetic acid (DTPA) and derivatives thereof, dimercaptopropane sulfonic acid (DMPS) and derivatives thereof, dimercaptosuccinic acid (DMSA) and derivatives thereof, aminotrimethylene phosphonic acid (ATPA) and derivatives thereof, N,N-bis(carboxymethyl)glycine (NTA) and derivatives thereof, nitrilotriacetic acid and derivatives thereof, citric acid and derivatives thereof, niacinamide and derivatives thereof, sodium desoxycholate and derivatives thereof, sodium phytate and derivatives thereof, trisodium ethylenediamine disuccinate and derivatives thereof, polyphosphates; porphine; and any pharmaceutically acceptable salts thereof.

The described chelating agents can be present in the described topical pharmaceutical compositions in an amount of, for example, from about 0.001 wt % to about 10 wt %; from about 0.005 wt % to about 5 wt %; from about 0.005 wt % to about 0.5 wt %; from about 0.001 wt % to about 1 wt %; from about 0.01 to about 5 wt %; from about 0.006 wt % to about 0.04 wt %; from about 0.007 wt % to about 0.035 wt %; from about 0.008 wt % to about 0.035 wt %; from about 0.009 wt % to about 0.035 wt %; from about 0.01 wt % to about 0.03 wt %; from about 0.015 wt % to about 0.025 wt %; from about 0.018 wt % to about 0.022 wt %; from about 0.019 wt % to about 0.021 wt %; about 0.019 wt %; about 0.02 wt %; or about 0.021 wt % chelating agent.

In one embodiment, the topical formulation is stable at room temperature. The optimal storage conditions are between 15° C. to 25° C., in a dry environment, and away from UV and heat.

Normal handling is done under good practices. Elevated temperatures are avoided.

### Kits for Use in Promoting Skin Health and Reducing the Effects of Aging

The present disclosure also provides kits for use in promoting skin health and reducing the effects of aging. Such kits may include one or more containers comprising a topical formulation as described herein, including but not limited to one or more compounds of Formula (I) or (X-I), and Tables 1 to 2 above.

In some embodiments, the kit may comprise instructions for use in accordance with any of the methods described herein. The included instructions may comprise a description of administration of the topical formulation to promote skin health and reducing the effects of aging according to any of the methods described herein. The kit may further comprise a description of selecting an individual suitable for treatment based on identifying whether that individual is in need of treatment. The instructions relating to the use of a topical formulation generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for promoting skin health and reducing the effects of aging. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. At least one active agent in the composition is an active agent selected from the group comprising compounds from the Tables 1 to 2.

Kits may optionally provide additional components such as interpretive information. Normally, the kit comprises a container and a label or package insert(s) on or associated with the container. In some embodiments, the invention provides articles of manufacture comprising contents of the kits described above.

### Methods of Use—Promoting Skin Health and Preventing or Reducing Inflammaging

“Inflammaging” as described herein refers to inflammation, particularly of skin, as a biological response of a body tissue to both environmental challenges such as sun and wind, and internal drivers such as diet, alcohol consumption and smoking, and other potentially harmful stimuli, that promotes detrimental biological responses that result in visible signs of aging such as the appearance of fine lines and wrinkles, hyperpigmentation and increased laxity. The harmful stimuli can include but are not limited to pathogens, bacteria, viruses, fungi, damaged cells and other irritants that are known to those skilled in the art. While inflammation can be a protective immune response that can involve, for example, immune cells, white blood cells, blood vessels, molecular mediators, and other small molecules. Signs of inflammation can include but is not limited to pain, heat, swelling, and/or loss of function. Inflammation can be acute or chronic.

In some embodiments described herein, a formulation is provided for the treatment of inflammaging. The formulation can include cells manufactured by the methods described herein.

In some embodiments, the subject suffers from or is at risk of developing inflammaging in one or more skin regions.

In some embodiments, the inflammation is on the skin, scalp, nasal passages, mouth, nail area such as the cuticles, eyes, vaginal area or the perineal area.

Any of the topical formulations described herein can be used for promoting skin health and reducing the effects of aging in a subject in need of the treatment. The topical formulation may be applied to a skin area following a suitable dosage and treatment regimen. The dosage and administration regimen for the described method is depend on the nature and condition of the wound being treated, the age and condition of the patient, and any prior or concurrent therapy.

In some instances, the topical formulation can be applied once every week, once every other day, once daily, twice daily, three times daily, or four time daily for a suitable period of time. The treatment may be terminated when the skin area is recovered. When necessary, the treatment may resume, for example, if a skin area needs additional treatment.

According to the invention, the topical formulations may be administered topically in the form of a cream, gel, or liquid. The topical administration provides the stabilized formulation directly to the skin, which is preferably provided with the use of a dermatologically acceptable carrier. While the carrier may consist of a relatively simple solvent or dispersant, such as an oil, it is generally preferred that the carrier comprise a material more conducive to topical application, and particularly one which will form a film or layer on the skin to which it is applied. This localizes the application and provides some resistance to perspiration and/or aids in percutaneous delivery and penetration of the active ingredients into lipid layers. Many such compositions are known in the art, and can take the form of creams, gels, ointments, hydrogels, pastes or plasters, and liquid dosage forms, such as solutions, emulsions, in particular oil-in-water emulsions, suspensions, for example lotions, etc., or even solid sticks. Liposomes or microspheres may also be used.

In some embodiments, the topical formulation may be administered using a device or method designed to more readily break the skin barrier and provide the agents in the topical formulation with a faster or more effective means through the stratum corneum. These include, for example, ultrasound therapy or ultrasound, oxygen nebulizers and nanosomal mist in conjunction with iontophoresis. In some embodiments, a spray or nebulizer may be used to create the nanosomel mist. In one embodiment, the micro-electronic cosmetic delivery mechanism described as PowerCosmetics® may be used for delivery of the topical formulation to the skin. This method is useful for delivering ionizable compounds to the skin and aids the penetration of small molecules through the stratum corneum.

In one embodiment, low intensity ultrasound delivery systems described as OZ Inside™ may be used for delivery of the topical formulation to the skin. PCT/US2011/041787, PCT/US2014/043951 are hereby incorporated by reference for using such delivery systems to administer the topical formulation of this disclosure.

The subject to be treated by the topical formulation can be a human or a non-human mammal.

In some embodiments, the subject is a human patient who would benefit from improvement of skin health, promotion of skin self-renewing processes and protection against oxidative damage to skin.

In some embodiments, a method for treating a subject suffering from a skin disorder is provided. The method can comprise providing the cell of any of the embodiments described herein or the topical formulation of any of the embodiments described herein and applying the topical formulation to the subject, wherein the topical formulation is applied onto skin.

In some embodiments, the skin disorder is selected from a group consisting of psoriasis, skin cancer, acne, alopecia, carbuncles, dermatitis, eczema, atopic dermatitis, contact dermatitis, seborrheic dermatitis, cradle cap, perioral dermatitis, shingles, ringworm, melisma, and impetigo.

In some embodiments, the skin disorder arises from an autoimmune or inflammatory disorder.

In some embodiments, the autoimmune or inflammatory disorder is Alopecia areata, autoimmune angioedema, Autoimmune progesterone dermatitis, Autoimmune urticarial, Bullous pemphigoid, Cicatricial pemphigoid, Dermatitis herpetiformis, Discoid lupus erythematosus, Epidermolysis bullosa acquisita, Erythema nodosum, Gestational pemphigoid, Hidradenitis suppurativa, Lichen planus, Lichen sclerosus, Linear IgA disease, Morphea, Pemphigus vulgaris, Pityriasis lichenoides et varioliformis acuta, Mucha-Habermann disease, Psoriasis, Systemic scleroderma or Vitiligo. In some embodiments, skin diseases and skin disorders can include acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriee des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer and tropical acne.

### Methods of Use

Provided herein are methods of treating a disease or disorder in a subject, comprising administering to the subject a therapeutically effective amount of a gp130 receptor modulator. In some embodiments, provided herein are methods of treating a disease or disorder in a subject, comprising administering to the subject a therapeutically effective amount of a gp130 receptor agonist or with an effective amount of a modulator of gp130 signaling pathway described herein.

Provided herein is a method of repairing a joint surface injury in a subject, comprising administering to the subject a therapeutically effective amount of a binding site 1 gp130 receptor agonist or with an effective amount of a modulator of gp130 signaling pathway described herein.

### Methods of Use—Proliferative Disorder

Provided herein are methods of treating a proliferative disorder in a subject, comprising administering to the subject a therapeutically effective amount of a gp130 receptor modulator. In some embodiments, the proliferative disorder is a cancer. In certain embodiments, the cancer is a cancer that is mediated in part by a gp130 receptor. In certain embodiments, the cancer is a cancer that is responsive to inhibition of a gp130 receptor.

In some embodiments, the cancer is a skin cancer. In some embodiments, the skin cancer is a melanoma, basal cell carcinoma, squamous cell carcinoma,, Kaposi sarcoma, angiosarcoma, cutaneous B-cell lymphoma, cutaneous T-cell lymphoma, dermatofibrosarcoma protuberans, Merkel cell carcinoma, or sebaceous carcinoma.

Merkel cell carcinoma, sebaceous carcinoma.

### Methods of Use—Cartilage

Also provided herein is a method of treating a cartilage degenerative disease in a subject in need thereof. The method includes administering to the subject a therapeutically effective amount of a binding site 1 gp130 receptor agonist or with an effective amount of a modulator described herein. In embodiments, the disorder is arthritis. In embodiments, the disorder is osteoarthritis. In embodiments, the disorder is rheumatoid arthritis.

Further provided herein are methods of increasing secretion of cartilaginous matrix in cartilage, including contacting a gp130 receptor with a gp130 receptor agonist or with an effective amount of a modulator described herein. In embodiments, methods of increasing secretion of cartilaginous matrix in cartilage, comprise contacting a gp130 receptor with a binding site 1 gp130 receptor agonist or with an effective amount of a modulator described herein.

In embodiments, the cartilaginous matrix is in articular cartilage. In embodiments, the cartilaginous matrix includes collagens and proteoglycans.

### Methods of Use—Modulating the Activity of a Gp130 Receptor

Provided herein is a method of modulating the activity of a gp130 receptor in a cell.

The method comprises contacting the cell with a binding site 1 gp130 receptor agonist or with an effective amount of a compound of formula (I), (X-I), (X-I-A), (X-I-B), (II), (III), (IV), (V), (V-a-j), (VI), (VIa-f), (VII), or (VIII) described herein.

In embodiments, the activity of the gp130 receptor is increased. In embodiments, the activity of the gp130 receptor is decreased or inhibited. In embodiments, the activity is heterodimerization.

Also provided herein is a method of transforming a mature adult cell to a progenitor cell, comprising contacting the cell with a binding site 1 gp130 receptor agonist or with an effective amount of a modulator described herein.

In embodiments, the cell is a human cell. In embodiments, the cell is a chondrocyte. In some embodiments, the chondrocyte is an adult chondrocyte. In some embodiments, the binding site 1 cgp130 receptor agonist is a compound described herein e.g., a compound of Formula (I), (X-I), (X-I-A), (X-I-B), (II), (III), (IV), (V), (V-a-j), (VI), (VIa-f), (VII), or (VIII) as described herein.

Provided herein is a method of regulating chondrocyte activation, maturation and/or differentiation, comprising contacting a chondrocyte with a modulator described herein.

Also provided herein a method of regenerating or repairing tissue in a subject in need thereof, comprising administering to the subject a therapeutically effective amount of a modulator described herein. Preferably, the tissue is cartilage.

Further provided herein are methods of modulating expression of COL2 and ACAN in the presence of IL-6 family cytokines.

Further provided herein are methods of modulating expression of MMP13 and ADAMTS4 in the presence of IL-6 family cytokines.

Further provided herein are methods of modulating activity of SRC, NF-κB, STAT3, p38, ERK1/2, YAP, or MYC, or a combination thereof in a cell.

In one aspect, the method includes contacting the cell with a binding site 1 gp130 receptor agonist or with an effective amount of a compound of formula (I), (X-I), (X-I-A), (X-I-B), (II), (III), (IV), (V), (V-a-j), (VI), (VIa-f), (VII), or (VIII) described herein.

The contacting may be performed in vitro. The contacting may be performed in vivo.

Provided herein is a method of regulating chondrocyte activation, maturation and/or differentiation. In certain aspects, the method includes contacting a chondrocyte with a binding site 1 gp130 receptor agonist or with an effective amount of a modulator described herein.

In some embodiments, the chondrocyte activation includes an increase in proliferation, migration, metabolism or any combination thereof.

The disclosure also provides a method of treating an inflammatory disease or disorder or cell-proliferative disease or disorder comprising contacting a subject with a compound as described herein and above. In one embodiment, the inflammatory disease or disorder or cell proliferative disease or disorder is selected from the group consisting of stroke; heart disease; cartilage degeneration; hair loss; wound healing; arthritis; fibrosis; neurodegenerative disorders; aging; diseases known to be associated with low grade chronic inflammation; immune disorders including psoriasis, rosacea, lupus, rheumatoid arthritis, inflammatory bowel disease; cytokine release syndrome; and cancer.

In some embodiments, the disclosure provides methods of modulating the production or induction of inflammation and/or inflammatory cytokines comprising contacting a cell or subject with compounds of formula (I), (X-I), (X-I-A), (X-I-B), (II), (III), (IV), (V), (V-a-j), (VI), (VIa-f), (VII), or (VIII) as described herein. In certain embodiments, the cell is a chondrocyte.

The disclosure also provides a method of modulating IL-6 mediated inflammatory responses in a cell comprising contacting the cell with a compound as described herein and above. In one embodiment, the cell is a chondrocyte.

The disclosure also provides a composition comprising a pharmaceutically acceptable carrier and a compound of formula (I), (X-I), (X-I-A), (X-I-B), (II), (III), (IV), (V), (V-a-j), (VI), (VIa-f), (VII), or (VIII) as described herein and above.

The disclosure also provides a method of treating an acute of chronic inflammatory state comprising contacting a subject with a compound of formula (I), (X-I), (X-I-A), (X—I-B), (II), (III), (IV), (V), (V-a-j), (VI), (VIa-f), (VII), or (VIII) or pharmaceutical as described herein and above.

The disclosure also provides a method of decreasing an activated inflammatory pathway in a cell comprising contacting the cell with a compound of formula (I), (X-I), (X-I-A), (X-I-B), (II), (III), (IV), (V), (V-a-j), (VI), (VIa-f), (VII), or (VIII) or pharmaceutical as described herein and above.

The disclosure provides a method of inhibiting the production or induction of pro-inflammatory genes, cytokines or mediators comprising contacting a cell or subject with a compound of formula (I), (X-I), (X-I-A), (X-I-B), (II), (III), (IV), (V), (V-a-j), (VI), (VIa-f), (VII), or (VIII) or pharmaceutical as described herein and above.

The disclosure provides a method of inhibiting the production or induction of extracellular matrix degrading enzymes comprising contacting a cell or subject with a compound of formula (I), (X-I), (X-I-A), (X-I-B), (II), (III), (IV), (V), (V-a-j), (VI), (VIa-f), (VII), or (VIII) or pharmaceutical as described herein and above.

The disclosure also provides a method treating an acute or chronic inflammatory state comprising contacting a subject with a compound of Formula (I).

The disclosure further provides a method of decreasing an activated inflammatory pathway in a cell comprising contacting the cell with a compound of Formula (I).

The disclosure provides a method of inhibiting the production or induction of pro-inflammatory genes, cytokines or mediators comprising contacting a cell or subject with a compound of Formula (I).

The disclosure also provides a method of treating a skin disorder comprising contacting a subject with a modulator as described herein and above. In one embodiment

In some embodiments, the subject is a human patient who would benefit from improvement of skin health, promotion of skin self-renewing processes and protection against oxidative damage to skin.

In some embodiments, a method for treating a subject suffering from a skin disorder is provided. The method can comprise providing the cell of any of the embodiments described herein or the topical formulation of any of the embodiments described herein and applying the topical formulation to the subject, wherein the topical formulation is applied onto skin.

In some embodiments, the skin disorder is selected from a group consisting of psoriasis, skin cancer, acne, alopecia, carbuncles, dermatitis, eczema, atopic dermatitis, contact dermatitis, seborrheic dermatitis, cradle cap, perioral dermatitis, shingles, ringworm, melisma, vitiligo and impetigo.

In some embodiments, the skin disorder arises from an autoimmune or inflammatory disorder.

In some embodiments, the autoimmune or inflammatory disorder is Alopecia areata, autoimmune angioedema, Autoimmune progesterone dermatitis, Autoimmune urticarial, Bullous pemphigoid, Cicatricial pemphigoid, Dermatitis herpetiformis, Discoid lupus erythematosus, Epidermolysis bullosa acquisita, Erythema nodosum, Gestational pemphigoid, Hidradenitis suppurativa, Lichen planus, Lichen sclerosus, Linear IgA disease, Morphea, Pemphigus vulgaris, Pityriasis lichenoides et varioliformis acuta, Mucha-Habermann disease, Psoriasis, Systemic scleroderma or Vitiligo. In some embodiments, skin diseases and skin disorders can include acne aestivalis (Mallorca acne), acne conglobate, acne cosmetica (cosmetic acne), acne fulminans (acute febrile ulcerative acne), acne keloidalis nuchae (acne keloidalis, dermatitis papillaris capillitii, folliculitis keloidalis, folliculitis keloidis nuchae, nuchal keloid acne), adult forehead with scattered red pimples, acne vulgaris, acne mechanica, acne medicamentosa, acne miliaris necrotica (acne varioliformis), acne vulgaris, acne with facial edema (solid facial edema), blepharophyma, erythrotelangiectatic rosacea (erythematotelangiectatic rosacea, vascular rosacea), excoriated acne (acne excoriee des jeunes filles, Picker's acne), glandular rosacea, gnathophyma, gram-negative rosacea, granulomatous facial dermatitis, adult male with a large, red, bulbous nose, rhinophyma, granulomatous perioral dermatitis, halogen acne, hidradenitis suppurativa (acne inversa, pyoderma fistulans significa, Verneuil's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum, neonatal cephalic pustulosis), occupational acne, oil acne, ocular rosacea (ophthalmic rosacea, ophthalmorosacea), otophyma, periorificial dermatitis, persistent edema of rosacea (chronic upper facial erythematous edema, Morbihan's disease, rosaceous lymphedema), phymatous rosacea, pomade acne, papulopustular rosacea (inflammatory rosacea), perifolliculitis capitis abscedens et suffodiens (dissecting cellulitois of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobate, synovitis-acne-pustulosis-hyperostosis-osteomyelitis syndrome (SAPHO syndrome), steroid rosacea, tar acne, skin cancer and tropical acne.

Further provided herein are methods of treating or ameliorating inflammaging.

In one aspect, the method treating or ameliorating inflammaging comprises contacting the cell of a subject with an effective amount of a modulator described herein.

In some embodiments, the subject suffers from or is at risk of developing inflammaging in one or more skin regions.

In some embodiments, the inflammation is on the skin, scalp, nasal passages, mouth, nail area such as the cuticles, eyes, vaginal area or the perineal area.

In one embodiment, a modulator of gp130 signaling pathway described herein can be used for promoting skin health and reducing the effects of aging in a subject in need of the treatment.

In one embodiment, a modulator of gp130 signaling pathway described herein can be used for promoting hair growth in a subject in need of the treatment.

In one embodiment, a modulator of gp130 signaling pathway described herein can be used for treating or ameliorating muscular dystrophy.

Further provided herein are methods of treating or ameliorating a pain condition in a subject in need thereof, wherein said pain condition is selected from the group consisting of: neuropathic pain, inflammatory pain, headache pain, somatic pain, visceral pain, musckulo-skeletal, craniofacial, other somatic forms of pain and referred pain.

In some embodiments, the inflammatory pain is selected from a pain associated with an inflammatory condition selected from the group consisting of arthritic disorder; autoimmune disease; connective tissue disorder; injury; infection; neuritis; and joint inflammation.

In some embodiments, the somatic pain is selected from the group consisting of excessive muscle tension; repetitive motion disorder; muscle disorder; myalgia; infection; and drugs.

The method includes administering to the subject a therapeutically effective amount of a binding site 1 gp130 receptor agonist or with an effective amount of a modulator described herein.

In another aspect, provided herein are methods of treating or ameliorating conditions comprising multiple syndromes selected from septic shock and cytokine storm.

### IL-6/gp130 Axis

The pathogenesis of osteoarthritis (OA) often begins from an injury to articular cartilage, which establishes chronic, low-grade inflammation mediated by interleukin-6/glycoprotein 130 (IL-6/gp130) and other factors that promote matrix degradation over time and eventual destruction of cartilage. IL-6 signaling through IL-6R/gp130 suppresses chondrocyte proliferation, promotes mineralization in articular cartilage, downregulation of matrix proteins and increases expression of matrix-degrading proteases. Moreover, blockade of IL-6 in vivo in mouse models of OA has been shown to be chondroprotective. Importantly, higher serum levels of IL-6 have been correlated with the development of OA in humans, and a monoclonal antibody against IL-6R is currently in Phase III clinical trials for the treatment of hand OA (NCT02477059).

Signaling downstream of IL-6/gp130 is mediated by multiple pathways, including signal transducer and activator of transcription 3 (STAT3). STAT3 has been demonstrated to have pleiotropic effects during chondrogenesis and in articular chondrocytes. During chondrogenic differentiation of multipotent mesenchymal stem cells, IL-6/STAT3 signaling promotes chondrocyte commitment and matrix production. Similarly, loss of STAT3 during limb formation results in increased hypertrophy, premature ossification and decreases in expression of the master regulator of chondrocyte identity SOX9. In contrast, in adult articular chondrocytes inhibition of STAT3 downstream of IL-6 is chondroprotective, reducing the severity of OA-like pathology in a mouse model. Together, these data indicate that IL-6/STAT3 signaling can drive matrix loss and development of OA in vivo in both mouse models and humans.

Recent studies have shown that Bone Morphogenetic Protein receptor IB (BMPR1B) marks superficial chondrocytes throughout human ontogeny and also in rodent joints. As described herein these cells can also be identified by their high level of IL-6 coreceptor gp130 expression and activity. Based on the known role of IL-6/gp130 signaling in hypertrophy and OA pathogenesis, a small molecule screen was performed to identify potential agents to manipulate gp130 signaling. These studies revealed Regulator of Cartilage Growth and Differentiation 423 (RCGD 423), a small molecule modulator of gp130 (see, e.g., PCT/US2016/020126, which is incorporated herein by reference for all purposes).

In vitro studies demonstrated that RCGD 423 signals through gp130 and interacts with the extracellular region of gp130.

Further elucidation of the mechanism of RCGD 423 demonstrated that it promotes the formation of active, ligand-independent gp130 homodimers, thereby distinguishing its activity from IL-6 family cytokines; this was reflected in critical differences in the downstream molecular events of IL-6 family cytokine and RCGD 423 stimulation. Moreover, RCGD 423 actively competes with signaling by pro-inflammatory IL-6 family cytokines by sequestering gp130 away from forming heterodimers with IL-6R. Finally, in a rat model of OA, this molecule evidenced a remarkable ability to prevent cartilage degeneration.

A close analog of RCGD 423 has been shown to stimulate hair cycle in mice through stabilization of MYC protein, thus validating the mechanism of action of this compound in a completely independent system. However, despite these positive results, increases in pSTAT3 and MYC levels may be detrimental in a clinical OA pathology scenario, based on potential pro-degenerative and oncogenic concerns, respectively.

RCGD 423 provided information regarding the specific regulatory pockets/clefts in gp130. Using this information, modeling and bench research led to the identification of other small molecules that interacted with the gp130 pockets/clefts. For example, one such molecule CX-011 (also referred to as “B8” herein) and related analogs are shown to be potent inhibitor of pro-catabolic signaling by IL-6 family cytokines and which do not affect levels of pSTAT3 or MYC protein (see, e.g., PCT/US2019/020058 and, which is incorporated herein by reference for all purposes).

CX-011 was predicted to bind gp130 in the same binding pocket as RCGD 423, and it is hypothesized that it stabilizes an inactive conformation. The in vitro results, suggest a small molecule inhibitor of pro-inflammatory, pro-degenerative signaling mediated by IL-6 family cytokines through gp130 would have great clinical importance. Although a biologic against IL-6R is currently being tested as a therapeutic against OA, this therapy does not block the effects of oncostatin M (OSM) and LIF, two other IL-6 family members with pro-catabolic consequences on articular cartilage. Thus, small molecule gp130 inhibitor such as CX-011 and analogs thereof are useful for post-traumatic OA and have a different method of action. It is hypothesized that broad modulation of IL-6 family cytokine signaling will interrupt the pro-inflammatory, pro-degenerative environment present post-injury.

Nevertheless, the physicochemical properties (e.g. solubility, potency, functional groups) and the sites of metabolic instability (“hot spots”) of RCGD 423, CX-01 and derivatives thereof needs to be improved to ensure successful clinical outcome. Significant gains in these properties could potentially result in a first-in-class for the treatment of post-traumatic OA. Therefore, this disclosure provides small molecule modulators of gp130 signaling pathway with improved properties. Particularly, these compounds are useful for treatment or amelioration of inflammatory disorders or conditions, neoplasms, and cell proliferative disorders.

### Examples

The present disclosure is further described in detail by reference to the following experimental examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention and are not to be construed as limiting in any way the remainder of the disclosure.

### Example 1: Creating a Library of Small Molecule 2p130 Signaling Pathway Modulators with Improved Properties

A library of structural variants based on the RCGD 423 and CX-011 (B805) scaffold was generated and they possess improved physical properties. The compounds of Formula (I) were found to have distinct molecular profile modulating different gp130 signaling pathways.

Compounds of Formula (I) or (X-I) include, but are not limited to, those shown in Tables 1 to 2. The compounds of Formula (I) or (X-I) can be synthesized by suitable synthesis method known in the art. The synthesis method may follow any organic synthesis route or a biocatalytic pathway.

### Example 2: Selection of 2p130 Signaling Pathway Modulator

The gp130 co-receptor LIFR is expressed on a subset of superficial chondrocytes throughout human ontogeny. Based on these data, it is hypothesized that gp130 would also be expressed on these cells, and this is indeed the case. Given the known (and mostly detrimental roles) of IL-6 family cytokines on articular chondrocytes, a high throughput screen is performed to identify small molecules that could potentially modulate gp130 signaling. In adult mice, IL-6/gp130 signaling drives Col10a1 expression and hypertrophy, which has been strongly associated with OA progression; compounds are thus screened for their ability to prevent increases in Col10-mCherry fluorescence in mouse limb mesenchymal cells stimulated with BMP-4, a strong driver of hypertrophy. Compounds are emerged after secondary screening as it could prevent increases in alkaline phosphatase, a marker of hypertrophy, in fetal articular chondrocytes.

Based on these data, three classes of compounds are identified:

Class 1: Pro-regenerative, anti-inflammatory. Inhibits activation of p38, ERK1/2 and NF-kB by OSM, as well as gp130 phosphorylation. At the same time, agonizes YAP activation by LIF while being permissive to STAT3 with LIF. It greatly inhibits MMP13 and ADAMTS4 expression induced by OSM.

Class 2: Primarily Pro-regenerative. Mild Inhibition of activation of p38, with little to no effect on ERK1/2 and NF-kB by OSM, as well as gp130 phosphorylation. At the same time, strongly agonizes YAP activation by LIF. It increases expression of COL2 and ACAN in the presence of OSM while inhibiting MMP13 but not ADAMTS4 expression induced by OSM.

Class 3: Primarily anti-inflammatory. Strong Inhibition of activation of p38, ERK1/2 and gp13-by OSM, NF-kB relatively low. At the same time, inhibits YAP and STAT3 activation by LIF. It inhibits MMP13 and ADAMTS4 expression induced by OSM. mRNA expression of COL2, ACAN; MMP13, and ADAMTS4 is evaluated using PCR.

These data identify distinct molecular profiles that affect gp130 signaling pathways. This supports the use of the present modulators for treating or ameliorating wide range of diseases, conditions, or disorders related to gp130 signaling.

### Example 3: Method of Manufacture of Formulated Compounds

The compounds disclosed herein are formulated according to cGMP requirements, as follows.
Weighing procedure: Drum lids and/or bags are wiped to avoid dust and debris contamination. All ingredients are weighed and measured and checked into separate, clean, properly identified suitable size tared container(s).
Compounding procedure: All equipment is cleaned and sanitized, as are all tools that come in contact with the product prior to use. All components are completely dry. Materials are then staged to the main blending area.

In Phase A, a first mixture is produced as follows. Into the main processing tank, equipped with a propeller mixer and side sweep, item #1 according to Table 2 is added and moderate speed mixing is begun. Items #2-#6 are then added in the order given, mixing well after each addition. The first mixture is mixed until completely uniform.

In Phase B, in a separate vessel, items #7 and #8 are premixed. Once item #8 has dissolved, the premix is slowly added to the first vessel processing tank and mixed until completely uniform.

In Phase C, Item #9 is added to a separate vessel and moderate speed mixing is begun. Items #10-#18 are added to the phase 3 vessel in the order given, with mixing after each addition. The formulation is mixed until uniform. Once uniform, the contents of the Phase C vessel are slowly added to the main processing tank and mixed until completely uniform.

In Phase D, a clean container is used to bring an adequate sample and the completed batch record to the laboratory for testing and approval.

TABLE 2
Master formulation

| | | Trade Name | % WT/WT |
|---|---|---|---|
| Item# | Phase | INCi Name | range |
| 1 | A | Gransil ORB-5CS | 23.75-26.25 |
| | | dimethicone, polysilicone-11, isohexadecane, | |
| | | ammonium polyacryloyldimethyl taurate, tocopheryl | |
| | | acetate, polysorbate 80, | |
| | | polysorbate 20 | |
| 2 | A | Armesil 12C | 7.6-8.4 |
| | | Isododecane | |
| 3 | A | Tween ® 20-LQ-(AP) | 0.855-0.945 |
| | | polysorbate 20 | |
| 4 | A | BV-OSC | 0.475-0.525 |
| | | tetrahexyldecyl ascorbate | |
| 5 | A | Frescolat ® X-Cool | 0.095-0.105 |
| | | menthyl ethylamido oxalate | |
| 6 | A | Frag. “Orange EO Blend CE-188609” | 0.09-0.21 |
| | | Limonene, citrus Aurantium Dulcis (Orange) peel oil, | |
| | | Citrus Tangerina (tangerine) peel oil | |
| 7 | B | DL-alpha tocopherol | 0.095-0.105 |
| | | tocopherol | |
| 8 | B | A gp130 signaling pathway modulator as set forth in | 0.0000285-0.00315 |
| | | Tables 1-2 | |
| 9 | C | Deionized Water | 48.0-54.0 |
| | | water (aqua) | Remainder |
| | | | Ingredient |
| 10 | C | Edela BD | 0.0475-0.1025 |
| | | Selected from one or more chelating agent: disodium | |
| | | EDTA, sodium phytate, and/or trisodium | |
| | | ethylenediamine disuccinate. | |
| 11 | C | 1,3-Butylene Glycol | 3.8-4.2 |
| | | butylene glycol | |
| 12 | C | Granthix APP | 2.375-2.625 |
| | | isohexadecane, ammonium polyacryloyldimethyl | |
| | | taurate, polysorbate 80 | |
| 13 | C | Hydrolite 5 Green | 0.95-1.05 |
| | | pentylene glycol | |
| 14 | C | Hylasome EG10 | 0.95-1.05 |
| | | water (Aqua), pentylene glycol, ethylhexylglycerin, | |
| | | sodium hyaluronate crosspolymer | |
| 15 | C | Matrixyl ® Morphomics ™ | 1.9-2.1 |
| | | water (aqua), pentylene glycol, capryly1 glycol, N- | |
| | | prolyl palmitoyl Tripeptide-56 acetate | |
| 16 | C | Borealine Expert | 0.095-1.105 |
| | | glycerin, Acer Rubrum extract | |
| 17 | C | Exfo-Bio | 1.9-2.1 |
| | | water (aqua), Spondias Mombin pulp extract, Mangifera | |
| | | Indica (mango) pulp extract, glycerin, Musa Sapientum | |
| | | (banana) pulp | |
| | | extract, benzyl alcohol, potassium sorbate | |
| 18 | C | Spectrastat G2N | 1.14-1.26 |
| | | glyceryl caprylate, glycerin, caprylhydroxamic acid | |

### Example 4: Clinical Evaluation of the Cosmetic Skin Care Formulation—Patient Selection

A single center clinical trial is conducted to assess the efficacy of the compounds and formulations disclosed herein when used over the course of 8 wees by women and men with mild to moderate photodamage on the face. The study assesses any statistically significant improvement in efficacy parameter clinical grading scores over the course of 8 weeks of use when compared with baseline scores. Changes are noted in any biomarkers associated with photoaging in skin samples taken from a subgroup.

### 1. Study Endpoints and Patients Randomization

Study Endpoints are as follows:
Clinical Grading of Efficacy Parameters at baseline and weeks 4 and 8.
VISIA Imaging Procedures are performed at baseline and week 8.
Antera 3D® Imaging Procedures at baseline and weeks 4 and 8, with image analysis for mean roughness (Ra), averaged melanin level, and wrinkles (indentation index and maximum depth) performed at the end of the study using images from baseline and weeks 4 and 8.
For a subgroup of at least 3 subjects, 2-mm biopsies are taken (1 at baseline and 1 at week 8; total of 2 per subject) and sent to Sponsor for histological evaluation.

### 2. Monitoring of Adverse Events (AEs) Throughout the Course of the Study.

Subjects are numbered sequentially in the order in which they qualify for entry into the study. All subjects use the test materials as instructed. A randomization is generated to establish the location of all Antera 3D® imaging (cheek and crow's feet) to the right or left side of the face.

### 3. Number of Subjects

At least 25 subjects meeting the eligibility requirements are expected to complete participation in the clinical trial, with at least 3 subjects in the biopsy subgroup.

### Eligibility Criteria: Inclusion Criteria

Individuals who meet all of the following criteria are eligible to participate in the study. Such individuals are:
Female or male, 30 to 65 years of age.
In generally good health (physical, mental, and social well-being, not merely the absence of disease/infirmity), according to subject self-report.
Having Fitzpatrick skin type I-V.

The Fitzpatrick skin classification is based on the skin's unprotected response to the first 30 to 45 minutes of sun exposure after a winter season without sun exposure. The categories of skin types are shown in Table 3.

TABLE 3
Skin Type categories

| | | |
|---|---|---|
| I | White; very fair; red or blonde hair; blue eyes; freckles | Always burns easily; never tans |
| II | White; fair; red or blonde hair; blue, hazel, or green | Always burns easily; tans |
| | eyes | minimally |
| III | Cream white; fair with any eye or hair color; very | Burns moderately; tans gradually |
| | common | |
| IV | Brown; typical Mediterranean white skin | Burns minimally; always tans |
| | | well |
| V | Dark brown; mid-eastern skin types, black hair, olive | Rarely burns; tans profusely |
| | skin | |
| VI | Black; black hair, black eyes, black skin | Never burns; deeply pigmented |

Having mild to moderate (score of 3-6 according to a modified Griffiths scale, 1 where 0=none and 9=severe) photodamage on the global face.
Having used a sunscreen with SPF≥30 for at least 30 days with no incident prior to study start, and willing to continue wearing this sunscreen for the duration of the study.
Willing to provide written informed consent and able to read, speak, write, and understand English.
Willing to sign a photography release.
Having not had any facial treatments in the past 6 months and are willing to withhold all facial treatments during the course of the study including facials, facial peels, photo facials, laser treatments, dermabrasion, botulinum toxin (Botox®), injectable filler treatments, intense pulsed light (IPL), acid treatments, tightening treatments, facial plastic surgery, or any other treatment administered by a physician or skin care professional designed to improve the appearance or firmness of facial skin. Waxing and threading are allowed but not facial laser hair removal.
Males who are regular facial shavers (at least 3 times per week). Subjects are allowed to have a mustache or small goatee as long as the majority of the face is shaved.
Willing to cooperate and participate by following study requirements (including those outlined in Example 3) for the duration of the study and to report any changes in health status or medications, AE symptoms, or reactions immediately.

The following additional inclusion criteria apply only to prospective subjects in the biopsy subgroup who are:
Willing to have two 2-mm punch biopsies taken from the face.
Willing to have blood drawn to screen for blood-borne pathogens including human immunodeficiency virus (HIV), hepatitis B (HBV), and hepatitis C (HCV).

### Eligibility Criteria: Exclusion Criteria

Individuals who meet any of the following criteria are not eligible to participate in the study, including individuals who:
are diagnosed with known allergies to facial skin care products;
are nursing, pregnant, or planning to become pregnant during the study according to subject self-report;
have a history of skin cancer within the past 5 years;
are currently using or have used any of the following medications within the noted time frame prior to the study start:
   1. Oral isotretinoin (Accutane®) within 6 months before the start of the study;
   2. Avita®, Differin®, Renova®, Retin-A®, Retin-A Micro®, Soriatane®, or Tazorac® within 3 months of the start of the study;
   3. Prescription-strength skin-lightening products (eg, hydroquinone, tretinoin, alpha/beta/poly-hydroxy acids, 4-hydroxyanisole alone or in combination with tretinoin, etc) within 3 months of the start of the study;
   4. Any anti-wrinkle, skin-lightening, or other product or topical or systemic medication known to affect skin aging or dyschromia (e.g., products containing alpha/beta/poly-hydroxy acids, emblica extract, Alpaflor® Gigawhite™, hydroquinone, lemon juice extract [topically], Q-10, soy, systemic or licorice extract [topically], Tego® Cosmo C₂₅₀, vitamin C) within 2 weeks of the start of the study;
have a health condition and/or pre-existing or dormant dermatologic disease on the face (e.g., psoriasis, rosacea, acne [severe acne, acne conglobata, nodules, or cysts], eczema, seborrheic dermatitis, severe excoriations) that the Investigator or designee deems inappropriate for participation or could interfere with the outcome of the study;
have observable sunburn, suntan, scars, nevi, excessive hair, tattoos, or other dermal conditions on the test sites that might influence the test results in the opinion of the Investigator or designee;
have a history of immunosuppression/immune deficiency disorders (including HIV infection, AIDS, multiple sclerosis, Crohn's disease, rheumatoid arthritis), organ transplant (heart, kidney, etc), or currently using oral or systemic immunosuppressive medications and biologics (eg, azathioprine, belimumab, Cimzia®, Cosentyx®, cyclophosphamide, cyclosporine, Enbrel®, Humira®, Imuran®, Kineret®, mycophenolate mofetil, methotrexate, Orencia®, prednisone, Remicade®, Rituxan®, Siliq™, Simponi®, Stelara®, Taltz®) and/or undergoing radiation or chemotherapy as determined by study documentation;
are currently using or having regularly used corticosteroids (systemic or topical, not nasal or ocular) within the past 4 weeks (including but not limited to betamethasone, clobetasol, desoximetasone, diflorasone, fluocinonide, fluticasone, mometasone, halcinonide, and halobetasol);
have a disease such as asthma, diabetes, epilepsy, hypertension, hyperthyroidism, or hypothyroidism that is not controlled by diet or medication. Individuals having multiple health conditions may be excluded from participation even if the conditions are controlled by diet, medication, etc.;
have started a long-term medication within the 2 months preceding the start of the study;
have any planned surgeries or invasive medical procedures during the course of the study. Non-invasive medical procedures or surgeries are reviewed for their impact on the study outcome and acceptability by the Investigator or designee;
are currently participating in any other clinical trial at the same clinical site, another research facility, or doctor's office;
have participated in any clinical trial involving the test area within 2 weeks prior to inclusion into the study at the same clinical site, at another research facility or doctor's office.
started hormone replacement therapies (HRT) or hormones for birth control less than 3 months prior to study entry or who plan on starting, stopping, or changing doses of HRT or hormones for birth control during the study; or
started prescription testosterone therapy less than 3 months prior to study entry or plan on starting, stopping, or changing doses of testosterone therapy during the study (e.g., testosterone cypionate, testosterone enanthate, testosterone undecanoate, and testosterone pellet) or on a testosterone booster or prescription testosterone (e.g., DHEA, tribulus, testosterone cypionate, testosterone enanthate, Sustanon, testosterone propionate, testosterone phenylpropriate, or Omnadren).

The following additional exclusions apply only to prospective subjects in the biopsy subgroup who:
have a medical history of allergy, hypersensitivity or any serious reaction to local antibiotic or antiseptic, local anesthesia, having any treatment which may affect the blood coagulation and hemostasis (anti-coagulant medications, including Coumadin, Heparin, Plavix, chronic NSAID use, etc.);
have a history of developing abnormal pigmentation responses (skin color changes) such as hyperpigmentation or hypopigmentation from medical procedures such as surgical incision and skin biopsies, or a history of healing defects such as hypertrophy or keloid scarring;
are currently or frequently using anti-inflammatory medication for a defined medical condition. Low dose aspirin (<81 mg per day) is acceptable;
have a history of systemic granulomatous diseases, active or inactive, (e.g., sarcoidosis, Wegener's granulomatosis, or tuberculosis) or connective tissue diseases (e.g., lupus or dermatomyositis); or
have been diagnosed with hepatitis or acute or chronic renal insufficiency.

### Example 5: Clinical Evaluation of the Skin Care Formulation—Treatment Regimen

Subjects are assigned a 3-digit number which, when used in conjunction with the clinical study number, is uniquely identify every subject on the study. This number remains with the subject throughout the study and should be used in all references to the individual in this study. No number is be reassigned once the study begins.

Subjects are Provided with the Following Instructions to Follow During the Study:

Pre-study instructions are to avoid application of facial moisturizer for at least 7 days prior to visit 1, and avoid application of any facial anti-wrinkle, skin-lightening, or other product or topical medication or treatment known to affect skin aging or dyschromia for at least 14 days prior to visit 1.

Test material usage instructions comprise the following: the test material is applied 2 times per day, morning and evening, before applying sunscreen; a quarter-sized amount per use is applied so that there is a complete layer on the skin; the entire face is covered, including under and around the eyes (avoiding the upper eyelids), around the mouth, along the jawline and close to ears; and 15 minutes waiting time before applying any other products on top of the serum.

Subject instructions for study visits comprise the following: the test material is applied as scheduled in the evening of the day prior to each post-baseline clinic visit. If the appointment time is in the morning (prior to noon), perform the morning application of the test material and cleanse the face at least 30 minutes prior to the site visit; wash the face and/or remove all makeup at least 30 minutes prior to each scheduled clinic visit. No other topical products are applied to the face or eye area until the study visit has been completed. If makeup is not removed prior to visiting the study site, makeup removal is required at the clinic and there is a waiting period of 20 minutes prior to test procedures.

General study instructions comprise the following: subject wears their sunscreen with SPF≥30 every day for the duration of the study. Extended periods of sun exposure are avoided as well as use of all tanning beds and sunless tanning products for the duration of the study. Extra care is taken to wear protective clothing and sunglasses and avoid sun exposure from 10 AM to 3 PM; the assigned test material is used as instructed; subject continues to use all regular brands of color cosmetics and makeup remover and uses the assigned test materials for the duration of the study. Subject refrains from using any antiaging products and does not start using any new facial products other than the assigned test materials.

Efficacy is assessed through clinical grading at baseline and weeks 4 and 8. VISIA imaging is performed at baseline and week 8. Antera 3D® digital imaging is performed at baseline and weeks 4 and 8, with image analysis performed at the end of the study on images from all time points. A subgroup of subjects has 2-mm punch biopsies between outer canthus and hairline (1 at baseline and 1 at week 8; total of 2 per biopsy subject). An outline of the procedures is shown in Table 4.

TABLE 4
Outline of Clinical Procedures

| | Visit 1 | | Visit 3 | Visit 4 |
|---|---|---|---|---|
| | Screening | | Week 4 | Week 8 |
| | (−10 to −3 | Visit 2 | (+/−3 | (+/−3 |
| Procedures/Time Points | days) | Baseline | days) | days) |
| ICF and qualification/enrollment CRFs | X | X | | |
| Blood draw for BBP screening | X | | | |
| Clinical Grading of Efficacy | | X | X | X |
| Parameters | | | | |
| VISIA Imaging Procedures | | X | | X |
| Antera 3D Imaging Procedures | | X | X | X |
| Biopsy Procedures (subgroup only) | | X | | X |
| Test Materials | | W/D | I/W | I/C/W |
| Daily Diaries | | D | C/R/D | C/R |

For test materials and/or daily diaries: D = Distribute, C = Collect, R = Review, W = Weigh, and I = Inspect (visually).
a A subgroup of subjects who may participate in biopsy procedures attends the screening visit.
b Antera 3D ® images from baseline and weeks 4 and 8 have image analysis for roughness, averaged melanin, and wrinkles after study completion.
c One 2-mm punch biopsy is taken at each indicated time point from each subject in a subgroup of subjects (at least 3) who have tested negative for BBP, with samples sent after study completion to Sponsor for histological evaluation.

### Screening for Prospective Biopsy Subjects: Visit 1

An IRB-approved informed consent form (ICF), consistent with the requirements in 21 Code of Federal Regulations (CFR) 50.25, is given to each prospective subject before participation in any study procedures. Prospective subjects are given as much time as needed to read the ICF and have the opportunity to have any study-related questions answered to their satisfaction prior to signing the ICF. If further questions exist, prospective subjects are given sufficient time during the first visit to have questions regarding the study and/or the ICF answered by the investigator, sub-investigator, or study coordinator prior to signing. An original signed ICF for each subject participating in the study is retained in the study file, and each subject receives a copy of the signed ICF. Prospective subjects are ineligible to participate in the study without a signed ICF.

Prospective biopsy subjects are given an IRB-approved ICF to read and sign. They have all of their study-related questions answered by the investigator or designated staff, and if they agree, they sign the ICF. They are given a copy of the signed ICF, and the original signed ICF is be kept in the study file.

Candidate subjects who sign the ICF are assigned a screening number and acclimate to ambient temperature and humidity conditions for at least 15 minutes.

Candidate subjects complete an eligibility and health questionnaire and are screened by the Investigator or designee for qualification criteria.

Candidate subjects (at least 3) who may have biopsies have blood samples drawn to screen for blood borne pathogens including HIV, HBV, and HCV.

### Baseline for Prospective Biopsy Subjects: Visit 2

A clinician records concomitant medications and asks candidate subjects if they have experienced any changes in their health since the previous visit. If an AE is reported, the investigator is informed, and an AE form is completed.

The qualified for biopsy sample collection candidate subjects have negative results for BBP including HIV, HBV, and HCV. Those who meet eligibility requirements are enrolled into the study and assigned a subject number.

Biopsy subjects acclimate to ambient temperature and humidity conditions for at least 15 minutes. The applicable rooms are maintained at a temperature of 68°−75° F. and the relative humidity is range from 35%-65%. Upon acclimation, candidate subjects participate in the following procedures: Clinical grading of efficacy parameters; VISIA® imaging procedures, and Antera 3D® imaging procedures. For qualified subjects, biopsies are be collected.

Each subject is provided with a pre-weighed unit of the test material and supporting material, and oral and written usage instructions. Subjects are provided with a daily diary to record test material applications and comments.

### Baseline, Visit 2: Non-Biopsy Subjects

Prospective subjects who sign the ICF are assigned a screening number and acclimate to ambient temperature and humidity conditions for at least 15 minutes. The applicable rooms are maintained at a temperature of 68°−75° F. and the relative humidity is range from 35%-65%.

Prospective subjects complete an eligibility and health questionnaire and are screened by the investigator or designee for qualification criteria.

Candidate subjects are graded for all efficacy parameters. Those who meet eligibility requirements are enrolled into the study and assigned a subject number. Subjects participate in VISIA imaging procedures and Antera 3D® imaging procedures. Each subject is provided with a pre-weighed unit of the test material and supporting material, and oral and written usage instructions.

### Subject Interim Visit: Visit 3 (Week 4±3 Days):

A clinician records concomitant medication, and asks candidate subjects, if they have experienced any changes in their health since the previous visit. If an AE is reported, the investigator is informed, and an AE form is be completed. Daily diaries are collected and reviewed for compliance. Subjects who are noncompliant to be counseled that, if they continue to be noncompliant, they are to be dropped from the study. Diaries are retained by the testing facility and new diaries are distributed to the subjects. Test material units are visually inspected and weighed to verify usage compliance. Test material units is returned to the subjects.

Subjects acclimate to ambient temperature and humidity conditions for at least 15 minutes. The applicable rooms are maintained at a temperature of 68°−75° F. and the relative humidity ranges from 35%-65%. Upon acclimation, subjects participate in clinical grading of efficacy parameters and Antera 3D® imaging procedures.

### Final Study Visit: Visit 4 (Week 8±3 Days)

A clinician records concomitant medication, and asks candidate subjects, if they have experienced any changes in their health since the previous visit. If an AE is reported, the investigator is informed, and an AE form is completed. Refer to the reporting procedures based on the section for AE below.

Daily diaries are collected, reviewed for compliance, and retained by the testing facility. Subjects who are noncompliant may be dropped from the study. Test material units are visually inspected and weighed to verify usage compliance. Test material units are retained by the testing facility. Subjects acclimate then to ambient temperature and humidity conditions for at least 15 minutes. The applicable rooms are maintained at a temperature of 68°−75° F. and the relative humidity range is from 35%-65%. Upon acclimation, subjects participate in the following procedures: Clinical grading of efficacy parameters, VISIA® imaging procedures, and Antera 3D® imaging procedures. Subjects in the biopsy subgroup is have biopsies collected.

### Example 6: Clinical Grading of Efficacy Parameters

Clinical grading of efficacy parameters is performed at baseline and weeks 4 and 8. The efficacy parameters are assessed globally on each subject's face using a modified Griffiths 10-point scale (Griffiths C E, Wang T S, Hamilton T A, Voorhees J J, Ellis C N. A photonumeric scale for the assessment of cutaneous photodamage. Arch Dermatol. 1992; 128(3):347-351) according to the following numerical definitions (half-point scores may be used as necessary to more accurately describe the skin condition):
0=none (best possible condition) 1 to 3=mild
4 to 6=moderate
7 to 9=severe (worst possible condition)

The following parameters are evaluated using the scale anchors indicated in Table 5.

TABLE 5
Scale Anchors for Evaluation of Images Using Griffiths 10 Point Scale

| Parameter | Location(s) | 0= | 9= |
|---|---|---|---|
| Fine lines | Global face | None | Numerous, deep fine |
| | | | lines |
| Wrinkles | Global face | None | Numerous, deep |
| | | | wrinkles |
| Smoothness (tactile) | Global face | Smooth, even-feeling | Rough, uneven-feeling |
| | | skin texture | skin texture |
| Mottled | Global face | Even skin color, no | Pronounced (dark, |
| hyperpigmentation | | hyperpigmentation | extensive) |
| | | | mottled/diffuse |
| | | | hyperpigmented areas |
| Radiance/luminosity/ | Global face | Radiant, luminous or | Dull/matte and/or |
| brightness | | glowing appearance | sallow skin appearance |
| Firmness/laxity | Global face | None | Severe laxity |

### 1. Digital Imaging Procedures

Prior to imaging procedures, clinic personnel ensure that subjects have a clean face with no makeup as described in the study procedures. Subjects remove any jewelry from the areas to be photographed and acclimate for at least 15 minutes to ambient conditions within the clinic before any photographs are taken. Subjects are provided with a black or gray matte headband to keep hair away from the face, and a black or gray matte cloth is be draped over the subjects' clothing.

Subjects are instructed to adopt neutral, nonsmiling expressions with their eyes gently closed, and are carefully positioned for each photograph.

### 2. VISIA® Imaging Procedures

VISIA imaging procedures are performed at baseline and week 8. A total of 3 full-face digital images are taken of each subject's face (left, center, and right views) using the VISIA CR photo station (Canfield Imaging Systems, Fairfield, New Jersey) with a Canon Mark II digital SLR camera (Canon Incorporated, Tokyo, Japan) under the following lighting conditions:
Standard lighting 1: visible (bright)
Standard lighting 2: visible Standard lighting 3
Cross-polarized Parallel polarized

Antera® 3D Imaging Procedures Antera 3D imaging procedures are performed on the cheek and crow's feet area (left or right side of face according to the randomization procedure generated by generated by Stephens) at baseline and weeks 4 and 8. A total of 2 images is taken per subject per time point. Each image is labeled with the study number, subject number, and site coding.

Photography is performed using the Antera 3D imaging (Miravex Limited, Dublin, Ireland). Antera 3D relies on multi-directional illumination and computer-aided reconstruction of the skin surface, illuminating the surface with light emitting diodes (LEDs) of different wavelengths shining from different angles and using the differences between these images to reconstruct the surface in three dimensions. The field of view is 56×56 mm with lateral resolution of 0.1 mm and vertical resolution 0.01 mm.

Images are evaluated according to selected parameters whereby the data for 30 patients shows the average change in clinical grading score in the designated parameters at 4 and 8 weeks.

### 3. Biopsy Procedures (Subgroup Only)

At baseline and week 8, a subgroup of subjects (at least 3) participates in biopsy procedures after all other procedures. Each selected subject has a 2-mm punch biopsy taken between outer canthus and hairline at baseline on the side of the face where Antera 3D imaging was performed. At week 8, the biopsy is taken between outer canthus and hairline on the other side of the face.

Biopsies are obtained using standard sterile technique after an intradermal local anesthesia and may be treated with topical antibiotic and sterile dressing in routine fashion. Test sites may be marked with a surgical marker for reference.

The biopsies are immediately transferred into 10% formalin solution and stored at room temperature for 12-16 hours, washed with tap water, and stored and shipped in 70% ethanol. Collected biopsy samples is be sent via FedEx to the Sponsor at the name and address below for histological evaluation. The biopsy sample manifest contains the subject number, product information, total number of biopsies collected, and any other relevant information needed to identify the samples post-analysis.

### Example 7: Synthesis of Compounds

### Preparation of N-(5-cyclopropyl-1,3,4-oxadiazol-2-yl)-1-ethyl-1H-pyrazole-3-carboxamide (Compound 7)

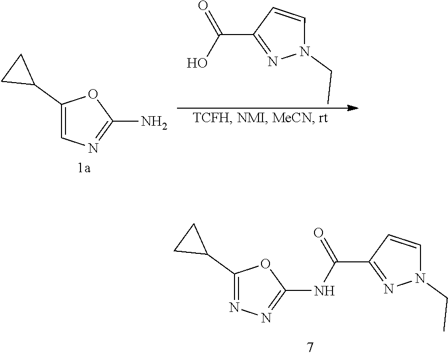

To a solution of 5-cyclopropyl-1,3,4-oxadiazol-2-amine 1a (100 mg, 0.80 mmol) and 1-ethylpyrazole-3-carboxylic acid (123 mg, 0.88 mmol) in ACN (10 mL) was added TCFH (336 mg, 1.20 mmol) and NMI (197 mg, 2.40 mmol). The resulting mixture was stirred at room temperature for 1 h. The resulting mixture was concentrated and purified on a Biotage Isolera One (C₁₈ column, eluting with 5% to 95% MeCN/H₂O containing 0.1% formic acid) and purified by prep-HPLC (column: Gemini 5um C₁₈ 150*21.2 mm, mobile phase: MeCN/H₂O containing 0.1% formic acid, gradient: 5% to 50%) to obtain N-(5-cyclopropyl-1,3,4-oxadiazol-2-yl)-1-ethyl-1H-pyrazole-3-carboxamide (Compound 7) (41.40 mg, 99% purity, 20.75% yield) as an off-white solid. ¹H NMR (400 MHz, DMSO-d₆, ppm) δ 11.34 (s, 1H), 7.93 (d, J=2.0 Hz, 1H), 6.84 (d, J=2.4 Hz, 1H), 4.24 (q, J=7.2 Hz, 2H), 2.28-2.07 (m, 1H), 1.43 (t, J=7.6 Hz, 3H), 1.16-0.92 (m, 4H). LCMS (ESI) calcd. for C₁₁H₁₄N₅O₂ [M+H]⁺ m/z 248.26, found 248.05

### Preparation of 1-methyl-N-(thiazolo[5,4-b]pyridin-2-yl)-1H-pyrazole-3-carboxamide (Compound 2)

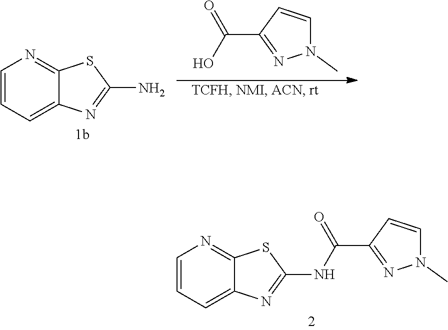

To a solution of thiazolo[5,4-b]pyridin-2-amine 1 (200 mg, 1.32 mmol) and 1-methylpyrazole-3-carboxylic acid (184 mg, 1.46 mmol) in ACN (10 mL) was added TCFH (557 mg, 1.98 mmol) and NMI (326 mg, 3.97 mmol). The resulting mixture was stirred at room temperature for 1 h. The resulting mixture was concentrated and purified on a Biotage Isolera One (C₁₈ column, eluting with 5% to 95% MeCN/H₂O containing 0.1% formic acid) to obtain 1-methyl-N-(thiazolo[5,4-b]pyridin-2-yl)-1H-pyrazole-3-carboxamide (Compound 2) (133.42 mg, 98% purity, 38.12% yield) as an off-white solid. ¹H NMR (400 MHz, DMSO-d₆, ppm) δ 12.51 (s, 1H), 8.49 (dd, J=4.8, 1.6 Hz, 1H), 8.14 (dd, J=8.0, 1.6 Hz, 1H), 7.93 (d, J=2.0 Hz, 1H), 7.51 (dd, J=8.0, 4.8 Hz, 1H), 7.06 (d, J=2.4 Hz, 1H), 3.99 (s, 3H). LCMS (ESI) calcd. for C₁₁H₁₀N₅OS [M+H]⁺ m/z 260.06, found 260.15.

### INCORPORATION BY REFERENCE

The entire disclosures of all patent and non-patent publications cited herein are each incorporated by reference in their entireties for all purposes.

### OTHER EMBODIMENTS

The disclosure set forth above may encompass multiple distinct disclosures with independent utility. The specific embodiments thereof as disclosed and illustrated herein are not to be considered in a limiting sense, because numerous variations are possible. The subject matter of the disclosures includes all novel and nonobvious combinations and subcombinations of the various elements, features, functions, and/or properties disclosed herein. The following claims particularly point out certain combinations and subcombinations regarded as novel and nonobvious. Disclosures embodied in other combinations and subcombinations of features, functions, elements, and/or properties may be claimed in this application, in applications claiming priority from this application, or in related applications. Such claims, whether directed to a different disclosure or to the same disclosure, and whether broader, narrower, equal, or different in scope in comparison to the original claims, also are regarded as included within the subject matter of the disclosures of the present disclosure.

1. A compound having a structure of Formula (X-I): 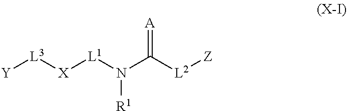
wherein:
A is O or S;
X is optionally substituted cycloalkyl, optionally substituted heterocyclyl, optionally substituted heteroaryl, or optionally substituted aryl;
Y is optionally substituted cycloalkyl, optionally substituted heterocyclyl, optionally substituted heteroaryl, or optionally substituted aryl, or is absent;
Z is optionally substituted cycloalkyl, optionally substituted heterocyclyl, optionally substituted heteroaryl, or optionally substituted aryl or optionally substituted alkyl, optionally substituted alkenyl, optionally substituted alkynyl;
L¹ is optionally substituted alkyl, optionally substituted alkenyl, alkynyl, optionally substituted cycloalkyl, optionally substituted cycloalkenyl, —O—, —S—, or —N(R^{1a})—, or is absent;
L² is optionally substituted alkyl, optionally substituted alkenyl, alkynyl, optionally substituted cycloalkyl, optionally substituted cycloalkenyl, —O—, —S—, or —N(R^{1a})—, or is absent;
L³ is optionally substituted alkyl, optionally substituted alkenyl, alkynyl, optionally substituted cycloalkyl, optionally substituted cycloalkenyl, —C(O)—, NH—C(O)—, —C(O)—NH—, —O—, —S—, or —N(R^{1a})—, or is absent;
R¹ is H or C₁-C₆ alkyl;
each R^{1a} is H or C₁-C₆ alkyl;
or
a pharmaceutically acceptable salt thereof.

2. The compound of claim 1, wherein the compound has a structure of Formula (X-I-A):

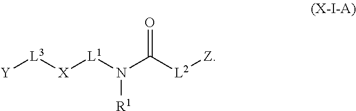

3. The compound of claim 1, wherein the compound has a structure of Formula (X-I-B):

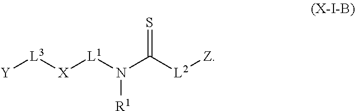

4. The compound of claim 1, wherein X is 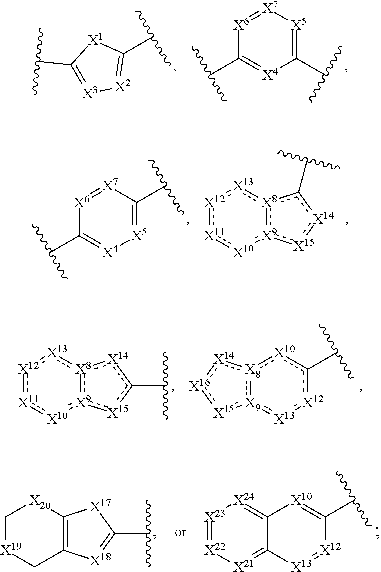
and Y is 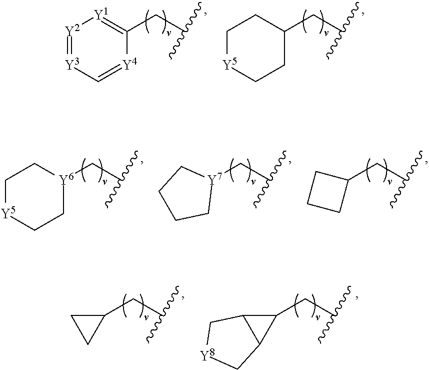
or is absent;
wherein,
X¹ to X¹⁸ are each independently —C(R²)=, —N═, —O—, or —S—;
X¹⁹ and X²⁰ are each independently —C(R²)(R³)—, —N(R⁴)—, —O—, or —S—;
Y¹, Y², Y³ and Y⁴ are each independently —C(R²)=, or —N═;
Y⁵ and Y⁸ are each independently —C(R²)(R³)—, —N(R⁴)—, —O—, or —S—;
Y⁶ and Y⁷ are each independently —C(R²)—, or N;
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, —S(O)₂N(H)(C₁-C₃)alkyl, —S(O)₂N(H)CH₃, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, 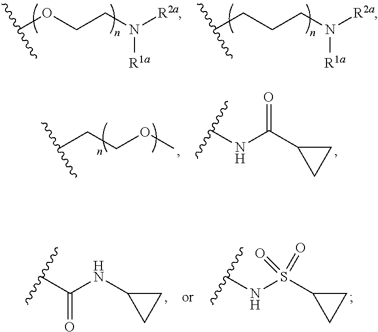
R⁴ is H, (C₁-C₃) alkyl, (C₁-C₃)haloalkyl, (C₁-C₃)alkenyl, (C₁-C₃)alkynyl, (C₁-C₃)alkoxyl, —S(O)₂N(H)(C₁-C₃)alkyl, —S(O)₂N(H)CH₃, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, 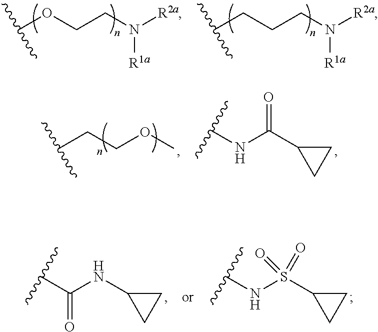
R^{1a} and R^{2a} are each independently H, (C₁-C₃) alkyl, (C₁-C₃)haloalkyl, (C₁-C₃)alkenyl, PGP-10 (C₁-C₃)alkynyl, or (C₁-C₃)alkoxyl, (C₃-C₆) branched alkyl, (C₃-C₆) branched haloalkyl, (C₃-C₆) branched alkenyl, (C₃-C₆) branched alkynyl, or (C₃-C₆) branched alkoxyl;
v is 0, 1 or 2; and
n is 0, 1, 2, 3, 4, or 5.

5. The compound of claim 1, wherein Z is 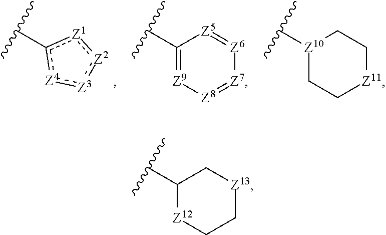
or is absent;
Z¹ to Z⁹ are each independently —C(R²)=, —N═, —O—, or —S—;
Z¹⁰ is —C(R²)— or N;
Z¹¹, Z¹² and Z¹³ are each independently —C(R²)(R³)—, —N(R⁴)—, —O—, or —S—.
R² and R³ are independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, —S(O)₂N(H)(C₁-C₃)alkyl, —S(O)₂N(H)CH₃, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, 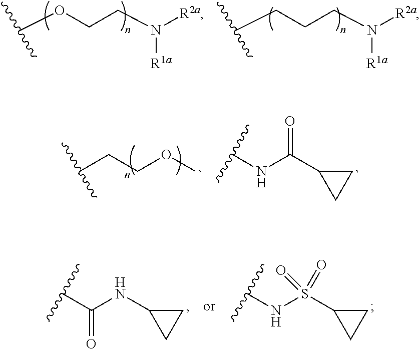
R⁴ is H, (C₁-C₃) alkyl, (C₁-C₃)haloalkyl, (C₁-C₃)alkenyl, (C₁-C₃)alkynyl, (C₁-C₃)alkoxyl, —S(O)₂N(H)(C₁-C₃)alkyl, —S(O)₂N(H)CH₃, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, 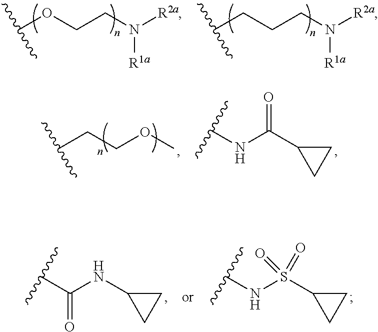
R^{1a} and R^{2a} are each independently H, (C₁-C₃) alkyl, (C₁-C₃)haloalkyl, (C₁-C₃)alkenyl, (C₁-C₃)alkynyl, (C₁-C₃)alkoxyl, (C₃-C₆) branched alkyl, (C₃-C₆) branched haloalkyl, (C₃-C₆) branched alkenyl, (C₃-C₆) branched alkynyl, or (C₃-C₆) branched alkoxyl; and
n is 0, 1, 2, 3, 4, or 5.

6. The compound of claim 1, wherein the compound has a structure of Formula (II): 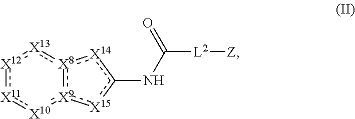
wherein,
X⁸ to X¹⁵ are each independently —C(R²)=, —N═, —O—, or —S—, and
R² is independently H, D, (C₁-C₃) alkyl, (C₁-C₃)haloalkyl, (C₁-C₃)alkenyl, (C₁-C₃)alkynyl, halo, cyano, hydroxyl, nitro, thiol, amino, (C₁-C₃)alkoxyl, —S(O)₂N(H)(C₁-C₃)alkyl, —S(O)₂N(H)CH₃, (C₃-C₆) branched alkyl, (C₃-C₆) branched haloalkyl, (C₃-C₆) branched alkenyl, (C₃-C₆) branched alkynyl, (C₃-C₆) branched alkoxyl, —S(O)₂N(H)(C₃-C₆)branched alkyl, 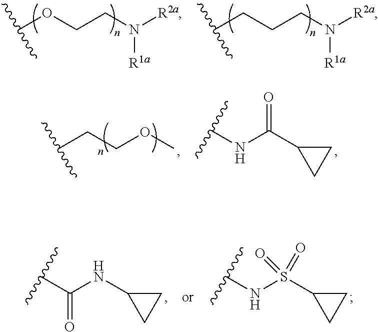
and
R^{1a} and R^{2a} are each independently H, (C₁-C₃) alkyl, (C₁-C₃)haloalkyl, (C₁-C₃)alkenyl, (C₁-C₃)alkynyl, or (C₁-C₃)alkoxyl, (C₃-C₆) branched alkyl, (C₃-C₆) branched haloalkyl, (C₃-C₆) branched alkenyl, (C₃-C₆) branched alkynyl, or (C₃-C₆) branched alkoxyl.

7. The compound of claim 1, wherein the compound has a structure of Formula (IIa):

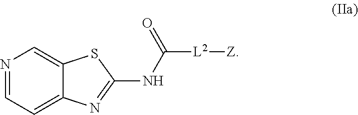

8. The compound of claim 1, wherein the compound has a structure of Formula (III):

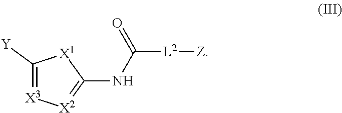

9. The compound of claim 1, wherein the compound has a structure of Formula (IIIa):

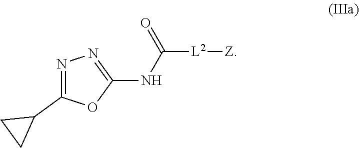

10. The compound of claim 1, wherein the compound has a structure of Formula (IV):

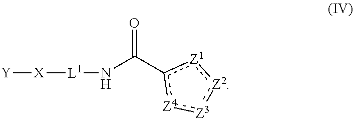

11. The compound of claim 1, wherein the compound has a structure of Formula (IVa):

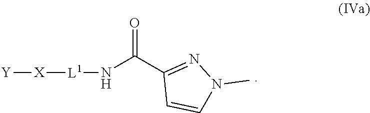

12. The compound of claim 1, wherein L¹ is absent.

13. The compound of claim 1, wherein L¹ is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —O—, —S—, or —N(R^{1a})—.

14. The compound of claim 1, wherein L² is absent.

15. The compound of claim 1, wherein L² is optionally substituted (C₁-C₃)alkyl, optionally substituted (C₁-C₃)alkenyl, (C₁-C₃)alkynyl, optionally substituted 5-6 membered cycloalkyl, optionally substituted 5-6 membered cycloalkenyl, —O—, —S—, or —N(R^{1a})—.

16. The compound of claim 1, wherein X is selected from the group consisting of:

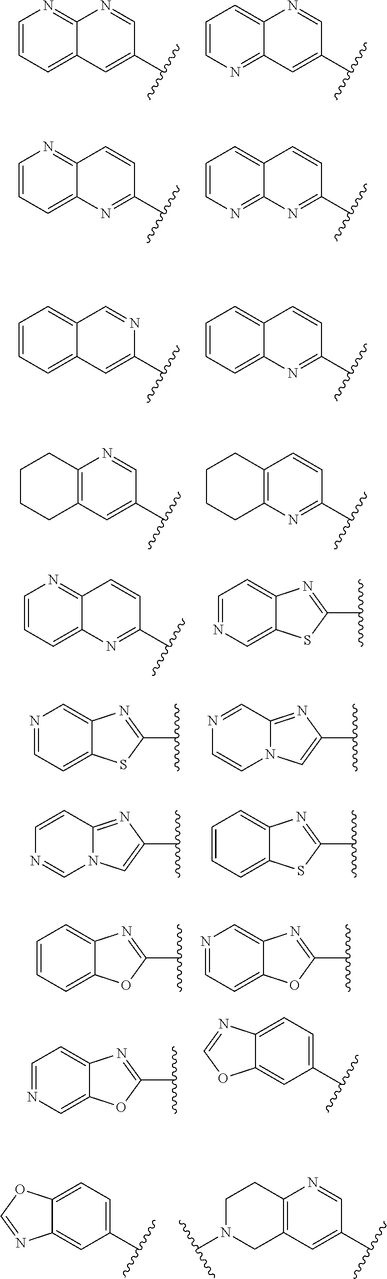

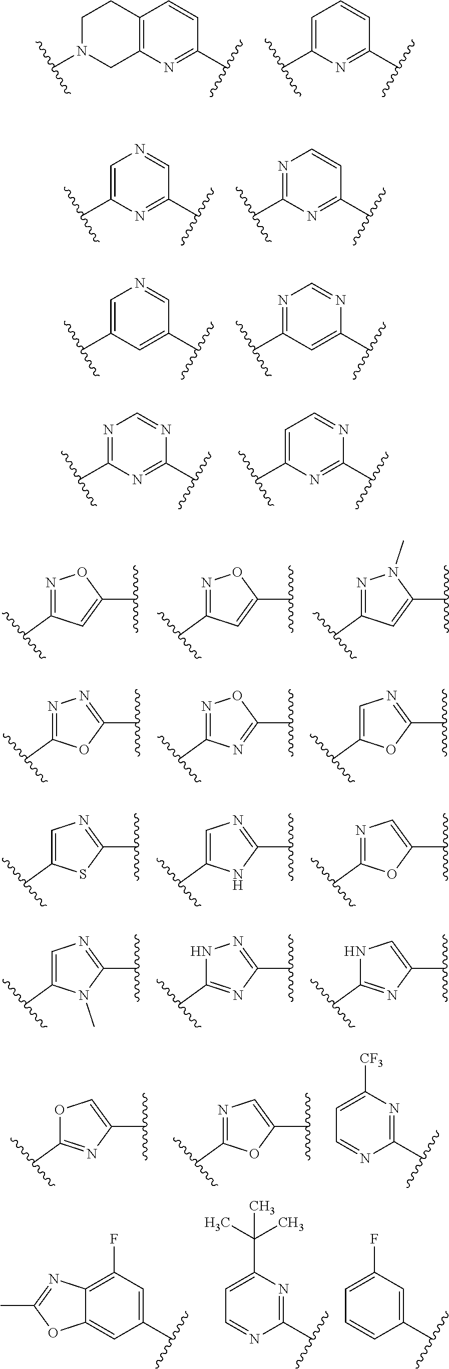

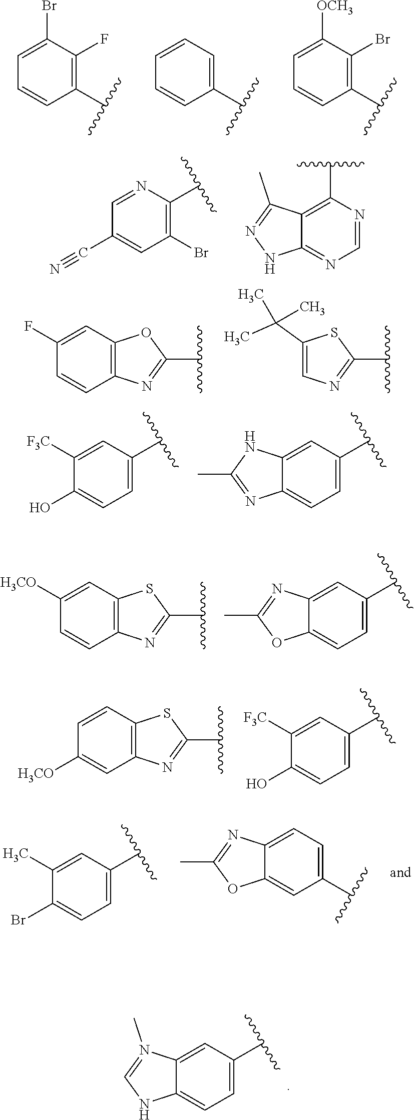

17. The compound of claim 1, wherein Z is selected from the group consisting of:

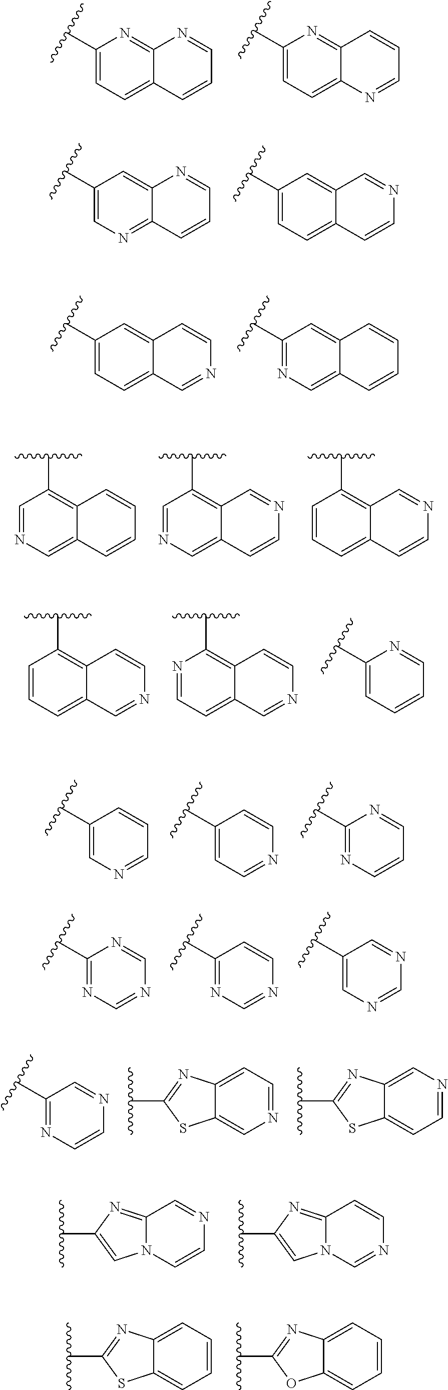

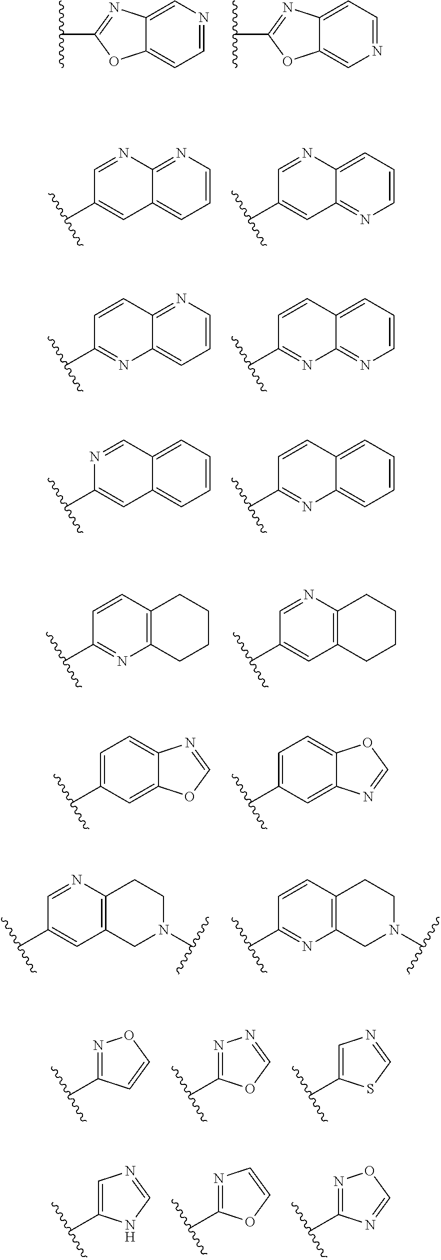

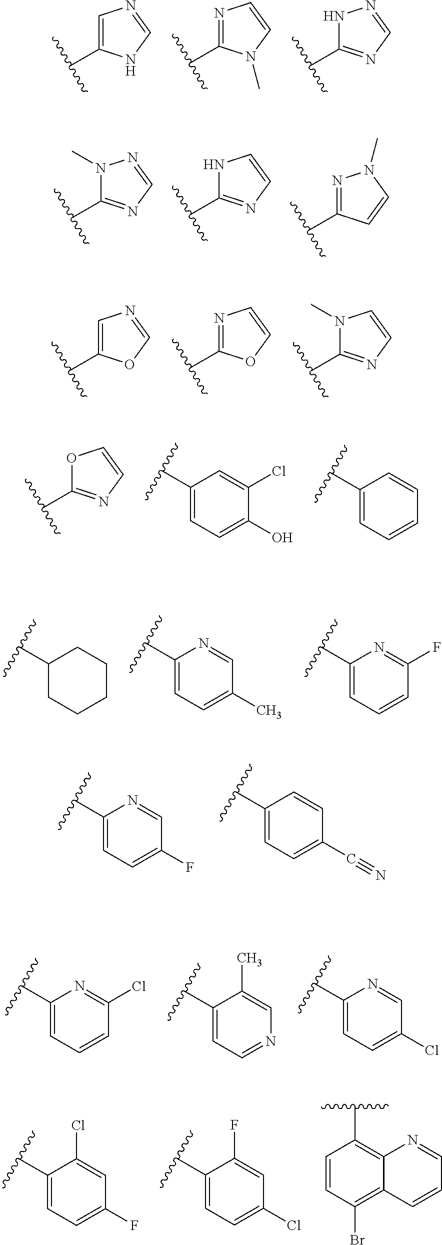

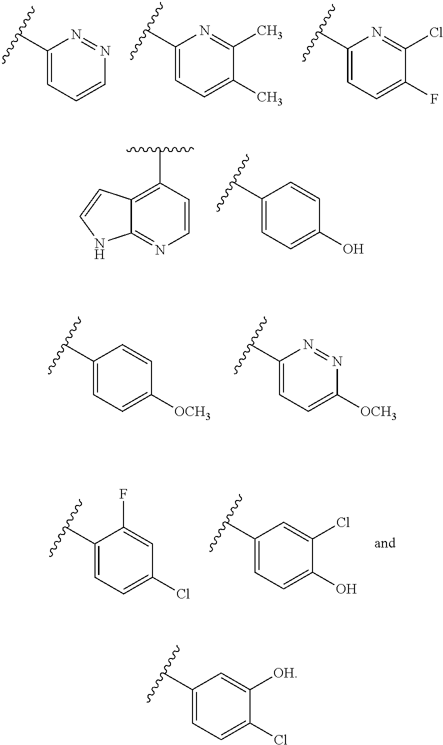

18. The compound of claim 1 wherein the compound is selected from a compound in Tables 1 to 2.

19. A pharmaceutical composition comprising one or more compounds of claim 1.

20-28. (canceled)

29. A topical formulation comprising a compound of claim 1.

30-46. (canceled)

47. A method of treating inflammation, inflammatory disease or disorder, reducing joint pain, preventing joint degeneration or promoting cartilage regeneration in a human subject in need thereof comprising administering to the subject an effective amount of a compound of claim 1.

48. The method of claim 47, wherein the subject is suffering from an inflammatory disease or disorder and is selected from the group consisting of stroke, heart disease, cartilage degeneration, hair loss, arthritis, neurodegenerative disorders, aging, psoriasis, rosacea, lupus, rheumatoid arthritis, inflammatory bowel disease, fibrosis, inflammaging and or chronic inflammation.

49. A method of treating a cell proliferative disease or disorder that is enhanced by gp130 activation in a human subject in need thereof comprising administering to the subject an effective amount of a compound of claim 1.

50. A method of treating or ameliorating a pain condition in a human subject in need thereof comprising administering to the subject an effective amount of a compound of claim 1, wherein said pain condition is selected from the group consisting of: neuropathic pain, inflammatory pain, headache pain, somatic pain, visceral pain, musckulo-skeletal, craniofacial, other somatic forms of pain and referred pain.

51. A method of modulating IL-6 family cytokine-mediated inflammatory responses in a cell comprising contacting the cell with a compound of claim 1.

52. (canceled)

53. A method of treating an acute or chronic inflammatory state comprising administering to a subject an effective amount of a compound of claim 1.

54-80. (canceled)

81. A kit comprising the compound of claim 1, or a pharmaceutically acceptable salt or solvate thereof, and instructions for administering the compound, or a pharmaceutically acceptable salt thereof, to a subject having an inflammatory disorder.
